SEQUENCE LISTING (469 sequences)

SEQ ID NO: 1 - DNA, 14 bp, synthetic construct
  source 1..14: mol_type = other DNA
gctttcacggaagg

SEQ ID NO: 2 - DNA, 14 bp, synthetic construct
  source 1..14: mol_type = other DNA
ctgcgcatcgggtg

SEQ ID NO: 3 - DNA, 14 bp, synthetic construct
  source 1..14: mol_type = other DNA
ccgcacttccgcga

SEQ ID NO: 4 - DNA, 14 bp, synthetic construct
  source 1..14: mol_type = other DNA
gcgccaagcggtgt

SEQ ID NO: 5 - RNA, 14 nt, synthetic construct
  source 1..14: mol_type = other RNA
atctcgcagcaccg

SEQ ID NO: 6 - RNA, 14 nt, synthetic construct
  source 1..14: mol_type = other RNA
cgcacttccgcgag

SEQ ID NO: 7 - RNA, 14 nt, synthetic construct
  source 1..14: mol_type = other RNA
atctcgcagcaccg

SEQ ID NO: 8 - RNA, 14 nt, synthetic construct
  source 1..14: mol_type = other RNA
gggagccccggcta

SEQ ID NO: 9 - DNA, 14 bp, synthetic construct
  source 1..14: mol_type = other DNA
cgaggtcacggcgt

SEQ ID NO: 10 - DNA, 14 bp, synthetic construct
  source 1..14: mol_type = other DNA
ccgcgtggtccgtc

SEQ ID NO: 11 - DNA, 14 bp, synthetic construct
  source 1..14: mol_type = other DNA
ggggcagcgtccca

SEQ ID NO: 12 - DNA, 14 bp, synthetic construct
  source 1..14: mol_type = other DNA
ccagaccttctggc

SEQ ID NO: 13 - RNA, 14 nt, synthetic construct
  source 1..14: mol_type = other RNA
caaagtgacccgag

SEQ ID NO: 14 - RNA, 14 nt, synthetic construct
  source 1..14: mol_type = other RNA
gggcatccgcagca

SEQ ID NO: 15 - DNA, 14 bp, synthetic construct
  source 1..14: mol_type = other DNA
tatgcagccttaat

SEQ ID NO: 16 - DNA, 14 bp, synthetic construct
  source 1..14: mol_type = other DNA
ctgccgcctggcca

SEQ ID NO: 17 - DNA, 14 bp, synthetic construct
  source 1..14: mol_type = other DNA
aaagtgacccgaga

SEQ ID NO: 18 - DNA, 14 bp, synthetic construct
  source 1..14: mol_type = other DNA
gctgcggatgcccc

SEQ ID NO: 19 - RNA, 14 nt, synthetic construct
  source 1..14: mol_type = other RNA
gtgacaccgagtgc

SEQ ID NO: 20 - RNA, 14 nt, synthetic construct
  source 1..14: mol_type = other RNA
agggcggaaaagtt

SEQ ID NO: 21 - RNA, 14 nt, synthetic construct
  source 1..14: mol_type = other RNA
aggccccggagtcc

SEQ ID NO: 22 - DNA, 14 bp, synthetic construct
  source 1..14: mol_type = other DNA
gggtagaccgtccc

SEQ ID NO: 23 - DNA, 14 bp, synthetic construct
  source 1..14: mol_type = other DNA
cgagtgcggggatc

SEQ ID NO: 24 - DNA, 14 bp, synthetic construct
  source 1..14: mol_type = other DNA
gcaccaccagagat

SEQ ID NO: 25 - DNA, 14 bp, synthetic construct
  source 1..14: mol_type = other DNA
caaccttctctcct

SEQ ID NO: 26 - RNA, 14 nt, synthetic construct
  source 1..14: mol_type = other RNA
ggggcctggtctcc

SEQ ID NO: 27 - RNA, 14 nt, synthetic construct
  source 1..14: mol_type = other RNA
actgcagccagggg

SEQ ID NO: 28 - DNA, 14 bp, synthetic construct
  source 1..14: mol_type = other DNA
tagggtgacttgaa

SEQ ID NO: 29 - DNA, 14 bp, synthetic construct
  source 1..14: mol_type = other DNA
acgaggtcgagccg

SEQ ID NO: 30 - DNA, 14 bp, synthetic construct
  source 1..14: mol_type = other DNA
ggcccattcatcaa

SEQ ID NO: 31 - DNA, 14 bp, synthetic construct
  source 1..14: mol_type = other DNA
gctcccttgcgccc

SEQ ID NO: 32 - DNA, 12 bp, synthetic construct
  source 1..12: mol_type = other DNA
gttttagagcta

SEQ ID NO: 33 - skipped

SEQ ID NO: 34 - DNA, 29 bp, synthetic construct
  source 1..29: mol_type = other DNA
ttttttttcagccaactccaatttttttt

SEQ ID NO: 35 - DNA, 24 bp, synthetic construct
  source 1..24: mol_type = other DNA
ccttcccgacttccgtaggagcgc

SEQ ID NO: 36 - DNA, 23 bp, synthetic construct
  source 1..23: mol_type = other DNA
ccggtgctccgcataccaagccg

SEQ ID NO: 37 - DNA, 24 bp, synthetic construct
  source 1..24: mol_type = other DNA
cctgaaggtaaagatcgggtcctc

SEQ ID NO: 38 - DNA, 24 bp, synthetic construct
  source 1..24: mol_type = other DNA
gcgctcctacggaagtcgggaagg

SEQ ID NO: 39 - DNA, 23 bp, synthetic construct
  source 1..23: mol_type = other DNA
cggcttggtatgcggagcaccgg

SEQ ID NO: 40 - DNA, 24 bp, synthetic construct
  source 1..24: mol_type = other DNA
gaggacccgatctttaccttcagg

SEQ ID NO: 41 - DNA, 83 bp, synthetic construct
  source 1..83: mol_type = other DNA
gctttcacggaagggttttagagctaccttcccgacttccgtaggagcgctttttttttgcgctcctacggaagtcgggaagg

SEQ ID NO: 42 - DNA, 83 bp, synthetic construct
  source 1..83: mol_type = other DNA
ctgcgcatcgggtggttttagagctaccttcccgacttccgtaggagcgctttttttttgcgctcctacggaagtcgggaagg

SEQ ID NO: 43 - DNA, 83 bp, synthetic construct
  source 1..83: mol_type = other DNA
ccgcacttccgcgagttttagagctaccttcccgacttccgtaggagcgctttttttttgcgctcctacggaagtcgggaagg

SEQ ID NO: 44 - DNA, 83 bp, synthetic construct
  source 1..83: mol_type = other DNA
gcgccaagcggtgtgttttagagctaccttcccgacttccgtaggagcgctttttttttgcgctcctacggaagtcgggaagg

SEQ ID NO: 45 - DNA, 81 bp, synthetic construct
  source 1..81: mol_type = other DNA
gctttcacggaagggttttagagctaccggtgctccgcataccaagccgtttttttttcggcttggtatgcggagcaccgg

SEQ ID NO: 46 - DNA, 81 bp, synthetic construct
  source 1..81: mol_type = other DNA
ctgcgcatcgggtggttttagagctaccggtgctccgcataccaagccgtttttttttcggcttggtatgcggagcaccgg

SEQ ID NO: 47 - DNA, 81 bp, synthetic construct
  source 1..81: mol_type = other DNA
ccgcacttccgcgagttttagagctaccggtgctccgcataccaagccgtttttttttcggcttggtatgcggagcaccgg

SEQ ID NO: 48 - DNA, 81 bp, synthetic construct
  source 1..81: mol_type = other DNA
gcgccaagcggtgtgttttagagctaccggtgctccgcataccaagccgtttttttttcggcttggtatgcggagcaccgg

SEQ ID NO: 49 - DNA, 83 bp, synthetic construct
  source 1..83: mol_type = other DNA
gctttcacggaagggttttagagctacctgaaggtaaagatcgggtcctctttttttttgaggacccgatctttaccttcagg

SEQ ID NO: 50 - DNA, 83 bp, synthetic construct
  source 1..83: mol_type = other DNA
ctgcgcatcgggtggttttagagctacctgaaggtaaagatcgggtcctctttttttttgaggacccgatctttaccttcagg

SEQ ID NO: 51 - DNA, 83 bp, synthetic construct
  source 1..83: mol_type = other DNA
ccgcacttccgcgagttttagagctacctgaaggtaaagatcgggtcctctttttttttgaggacccgatctttaccttcagg

SEQ ID NO: 52 - DNA, 83 bp, synthetic construct
  source 1..83: mol_type = other DNA
gcgccaagcggtgtgttttagagctacctgaaggtaaagatcgggtcctctttttttttgaggacccgatctttaccttcagg

SEQ ID NO: 53 - DNA, 83 bp, synthetic construct
  source 1..83: mol_type = other DNA
cgaggtcacggcgtgttttagagctaccttcccgacttccgtaggagcgctttttttttgcgctcctacggaagtcgggaagg

SEQ ID NO: 54 - DNA, 83 bp, synthetic construct
  source 1..83: mol_type = other DNA
ccgcgtggtccgtcgttttagagctaccttcccgacttccgtaggagcgctttttttttgcgctcctacggaagtcgggaagg

SEQ ID NO: 55 - DNA, 83 bp, synthetic construct
  source 1..83: mol_type = other DNA
ggggcagcgtcccagttttagagctaccttcccgacttccgtaggagcgctttttttttgcgctcctacggaagtcgggaagg

SEQ ID NO: 56 - DNA, 83 bp, synthetic construct
  source 1..83: mol_type = other DNA
ccagaccttctggcgttttagagctaccttcccgacttccgtaggagcgctttttttttgcgctcctacggaagtcgggaagg

SEQ ID NO: 57 - DNA, 81 bp, synthetic construct
  source 1..81: mol_type = other DNA
cgaggtcacggcgtgttttagagctaccggtgctccgcataccaagccgtttttttttcggcttggtatgcggagcaccgg

SEQ ID NO: 58 - DNA, 81 bp, synthetic construct
  source 1..81: mol_type = other DNA
ccgcgtggtccgtcgttttagagctaccggtgctccgcataccaagccgtttttttttcggcttggtatgcggagcaccgg

SEQ ID NO: 59 - DNA, 81 bp, synthetic construct
  source 1..81: mol_type = other DNA
ggggcagcgtcccagttttagagctaccggtgctccgcataccaagccgtttttttttcggcttggtatgcggagcaccgg

SEQ ID NO: 60 - DNA, 81 bp, synthetic construct
  source 1..81: mol_type = other DNA
ccagaccttctggcgttttagagctaccggtgctccgcataccaagccgtttttttttcggcttggtatgcggagcaccgg

SEQ ID NO: 61 - DNA, 83 bp, synthetic construct
  source 1..83: mol_type = other DNA
cgaggtcacggcgtgttttagagctacctgaaggtaaagatcgggtcctctttttttttgaggacccgatctttaccttcagg

SEQ ID NO: 62 - DNA, 83 bp, synthetic construct
  source 1..83: mol_type = other DNA
ccgcgtggtccgtcgttttagagctacctgaaggtaaagatcgggtcctctttttttttgaggacccgatctttaccttcagg

SEQ ID NO: 63 - DNA, 83 bp, synthetic construct
  source 1..83: mol_type = other DNA
ggggcagcgtcccagttttagagctacctgaaggtaaagatcgggtcctctttttttttgaggacccgatctttaccttcagg

SEQ ID NO: 64 - DNA, 83 bp, synthetic construct
  source 1..83: mol_type = other DNA
ccagaccttctggcgttttagagctacctgaaggtaaagatcgggtcctctttttttttgaggacccgatctttaccttcagg

SEQ ID NO: 65 - DNA, 83 bp, synthetic construct
  source 1..83: mol_type = other DNA
tatgcagccttaatgttttagagctaccttcccgacttccgtaggagcgctttttttttgcgctcctacggaagtcgggaagg

SEQ ID NO: 66 - DNA, 83 bp, synthetic construct
  source 1..83: mol_type = other DNA
ctgccgcctggccagttttagagctaccttcccgacttccgtaggagcgctttttttttgcgctcctacggaagtcgggaagg

SEQ ID NO: 67 - DNA, 83 bp, synthetic construct
  source 1..83: mol_type = other DNA
aaagtgacccgagagttttagagctaccttcccgacttccgtaggagcgctttttttttgcgctcctacggaagtcgggaagg

SEQ ID NO: 68 - DNA, 83 bp, synthetic construct
  source 1..83: mol_type = other DNA
gctgcggatgccccgttttagagctaccttcccgacttccgtaggagcgctttttttttgcgctcctacggaagtcgggaagg

SEQ ID NO: 69 - DNA, 81 bp, synthetic construct
  source 1..81: mol_type = other DNA
tatgcagccttaatgttttagagctaccggtgctccgcataccaagccgtttttttttcggcttggtatgcggagcaccgg

SEQ ID NO: 70 - DNA, 81 bp, synthetic construct
  source 1..81: mol_type = other DNA
ctgccgcctggccagttttagagctaccggtgctccgcataccaagccgtttttttttcggcttggtatgcggagcaccgg

SEQ ID NO: 71 - DNA, 81 bp, synthetic construct
  source 1..81: mol_type = other DNA
aaagtgacccgagagttttagagctaccggtgctccgcataccaagccgtttttttttcggcttggtatgcggagcaccgg

SEQ ID NO: 72 - DNA, 81 bp, synthetic construct
  source 1..81: mol_type = other DNA
gctgcggatgccccgttttagagctaccggtgctccgcataccaagccgtttttttttcggcttggtatgcggagcaccgg

SEQ ID NO: 73 - DNA, 83 bp, synthetic construct
  source 1..83: mol_type = other DNA
tatgcagccttaatgttttagagctacctgaaggtaaagatcgggtcctctttttttttgaggacccgatctttaccttcagg

SEQ ID NO: 74 - DNA, 83 bp, synthetic construct
  source 1..83: mol_type = other DNA
ctgccgcctggccagttttagagctacctgaaggtaaagatcgggtcctctttttttttgaggacccgatctttaccttcagg

SEQ ID NO: 75 - DNA, 83 bp, synthetic construct
  source 1..83: mol_type = other DNA
aaagtgacccgagagttttagagctacctgaaggtaaagatcgggtcctctttttttttgaggacccgatctttaccttcagg

SEQ ID NO: 76 - DNA, 83 bp, synthetic construct
  source 1..83: mol_type = other DNA
gctgcggatgccccgttttagagctacctgaaggtaaagatcgggtcctctttttttttgaggacccgatctttaccttcagg

SEQ ID NO: 77 - DNA, 83 bp, synthetic construct
  source 1..83: mol_type = other DNA
gggtagaccgtcccgttttagagctaccttcccgacttccgtaggagcgctttttttttgcgctcctacggaagtcgggaagg

SEQ ID NO: 78 - DNA, 83 bp, synthetic construct
  source 1..83: mol_type = other DNA
cgagtgcggggatcgttttagagctaccttcccgacttccgtaggagcgctttttttttgcgctcctacggaagtcgggaagg

SEQ ID NO: 79 - DNA, 83 bp, synthetic construct
  source 1..83: mol_type = other DNA
gcaccaccagagatgttttagagctaccttcccgacttccgtaggagcgctttttttttgcgctcctacggaagtcgggaagg

SEQ ID NO: 80 - DNA, 83 bp, synthetic construct
  source 1..83: mol_type = other DNA
caaccttctctcctgttttagagctaccttcccgacttccgtaggagcgctttttttttgcgctcctacggaagtcgggaagg

SEQ ID NO: 81 - DNA, 81 bp, synthetic construct
  source 1..81: mol_type = other DNA
gggtagaccgtcccgttttagagctaccggtgctccgcataccaagccgtttttttttcggcttggtatgcggagcaccgg

SEQ ID NO: 82 - DNA, 81 bp, synthetic construct
  source 1..81: mol_type = other DNA
cgagtgcggggatcgttttagagctaccggtgctccgcataccaagccgtttttttttcggcttggtatgcggagcaccgg

SEQ ID NO: 83 - DNA, 81 bp, synthetic construct
  source 1..81: mol_type = other DNA
gcaccaccagagatgttttagagctaccggtgctccgcataccaagccgtttttttttcggcttggtatgcggagcaccgg

SEQ ID NO: 84 - DNA, 81 bp, synthetic construct
  source 1..81: mol_type = other DNA
caaccttctctcctgttttagagctaccggtgctccgcataccaagccgtttttttttcggcttggtatgcggagcaccgg

SEQ ID NO: 85 - DNA, 83 bp, synthetic construct
  source 1..83: mol_type = other DNA
gggtagaccgtcccgttttagagctacctgaaggtaaagatcgggtcctctttttttttgaggacccgatctttaccttcagg

SEQ ID NO: 86 - DNA, 83 bp, synthetic construct
  source 1..83: mol_type = other DNA
cgagtgcggggatcgttttagagctacctgaaggtaaagatcgggtcctctttttttttgaggacccgatctttaccttcagg

SEQ ID NO: 87 - DNA, 83 bp, synthetic construct
  source 1..83: mol_type = other DNA
gcaccaccagagatgttttagagctacctgaaggtaaagatcgggtcctctttttttttgaggacccgatctttaccttcagg

SEQ ID NO: 88 - DNA, 83 bp, synthetic construct
  source 1..83: mol_type = other DNA
caaccttctctcctgttttagagctacctgaaggtaaagatcgggtcctctttttttttgaggacccgatctttaccttcagg

SEQ ID NO: 89 - DNA, 83 bp, synthetic construct
  source 1..83: mol_type = other DNA
tagggtgacttgaagttttagagctaccttcccgacttccgtaggagcgctttttttttgcgctcctacggaagtcgggaagg

SEQ ID NO: 90 - DNA, 83 bp, synthetic construct
  source 1..83: mol_type = other DNA
acgaggtcgagccggttttagagctaccttcccgacttccgtaggagcgctttttttttgcgctcctacggaagtcgggaagg

SEQ ID NO: 91 - DNA, 83 bp, synthetic construct
  source 1..83: mol_type = other DNA
ggcccattcatcaagttttagagctaccttcccgacttccgtaggagcgctttttttttgcgctcctacggaagtcgggaagg

SEQ ID NO: 92 - DNA, 83 bp, synthetic construct
  source 1..83: mol_type = other DNA
gctcccttgcgcccgttttagagctaccttcccgacttccgtaggagcgctttttttttgcgctcctacggaagtcgggaagg

SEQ ID NO: 93 - DNA, 81 bp, synthetic construct
  source 1..81: mol_type = other DNA
tagggtgacttgaagttttagagctaccggtgctccgcataccaagccgtttttttttcggcttggtatgcggagcaccgg

SEQ ID NO: 94 - DNA, 81 bp, synthetic construct
  source 1..81: mol_type = other DNA
acgaggtcgagccggttttagagctaccggtgctccgcataccaagccgtttttttttcggcttggtatgcggagcaccgg

SEQ ID NO: 95 - DNA, 81 bp, synthetic construct
  source 1..81: mol_type = other DNA
ggcccattcatcaagttttagagctaccggtgctccgcataccaagccgtttttttttcggcttggtatgcggagcaccgg

SEQ ID NO: 96 - DNA, 81 bp, synthetic construct
  source 1..81: mol_type = other DNA
gctcccttgcgcccgttttagagctaccggtgctccgcataccaagccgtttttttttcggcttggtatgcggagcaccgg

SEQ ID NO: 97 - DNA, 83 bp, synthetic construct
  source 1..83: mol_type = other DNA
tagggtgacttgaagttttagagctacctgaaggtaaagatcgggtcctctttttttttgaggacccgatctttaccttcagg

SEQ ID NO: 98 - DNA, 83 bp, synthetic construct
  source 1..83: mol_type = other DNA
acgaggtcgagccggttttagagctacctgaaggtaaagatcgggtcctctttttttttgaggacccgatctttaccttcagg

SEQ ID NO: 99 - DNA, 83 bp, synthetic construct
  source 1..83: mol_type = other DNA
ggcccattcatcaagttttagagctacctgaaggtaaagatcgggtcctctttttttttgaggacccgatctttaccttcagg

SEQ ID NO: 100 - DNA, 83 bp, synthetic construct
  source 1..83: mol_type = other DNA
gctcccttgcgcccgttttagagctacctgaaggtaaagatcgggtcctctttttttttgaggacccgatctttaccttcagg

SEQ ID NO: 101 - DNA, 103 bp, synthetic construct
  source 1..103: mol_type = other DNA
gctttcacggaagggttttagagctaccttcccgacttccgtaggagcgcttttttttcagccaactccaattttttttgcgctcctacggaagtcgggaagg

SEQ ID NO: 102 - DNA, 103 bp, synthetic construct
  source 1..103: mol_type = other DNA
ctgcgcatcgggtggttttagagctaccttcccgacttccgtaggagcgcttttttttcagccaactccaattttttttgcgctcctacggaagtcgggaagg

SEQ ID NO: 103 - DNA, 103 bp, synthetic construct
  source 1..103: mol_type = other DNA
ccgcacttccgcgagttttagagctaccttcccgacttccgtaggagcgcttttttttcagccaactccaattttttttgcgctcctacggaagtcgggaagg

SEQ ID NO: 104 - DNA, 103 bp, synthetic construct
  source 1..103: mol_type = other DNA
gcgccaagcggtgtgttttagagctaccttcccgacttccgtaggagcgcttttttttcagccaactccaattttttttgcgctcctacggaagtcgggaagg

SEQ ID NO: 105 - DNA, 101 bp, synthetic construct
  source 1..101: mol_type = other DNA
gctttcacggaagggttttagagctaccggtgctccgcataccaagccgttttttttcagccaactccaattttttttcggcttggtatgcggagcaccgg

SEQ ID NO: 106 - DNA, 101 bp, synthetic construct
  source 1..101: mol_type = other DNA
ctgcgcatcgggtggttttagagctaccggtgctccgcataccaagccgttttttttcagccaactccaattttttttcggcttggtatgcggagcaccgg

SEQ ID NO: 107 - DNA, 101 bp, synthetic construct
  source 1..101: mol_type = other DNA
ccgcacttccgcgagttttagagctaccggtgctccgcataccaagccgttttttttcagccaactccaattttttttcggcttggtatgcggagcaccgg

SEQ ID NO: 108 - DNA, 101 bp, synthetic construct
  source 1..101: mol_type = other DNA
gcgccaagcggtgtgttttagagctaccggtgctccgcataccaagccgttttttttcagccaactccaattttttttcggcttggtatgcggagcaccgg

SEQ ID NO: 109 - DNA, 103 bp, synthetic construct
  source 1..103: mol_type = other DNA
gctttcacggaagggttttagagctacctgaaggtaaagatcgggtcctcttttttttcagccaactccaattttttttgaggacccgatctttaccttcagg

SEQ ID NO: 110 - DNA, 103 bp, synthetic construct
  source 1..103: mol_type = other DNA
ctgcgcatcgggtggttttagagctacctgaaggtaaagatcgggtcctcttttttttcagccaactccaattttttttgaggacccgatctttaccttcagg

SEQ ID NO: 111 - DNA, 103 bp, synthetic construct
  source 1..103: mol_type = other DNA
ccgcacttccgcgagttttagagctacctgaaggtaaagatcgggtcctcttttttttcagccaactccaattttttttgaggacccgatctttaccttcagg

SEQ ID NO: 112 - DNA, 103 bp, synthetic construct
  source 1..103: mol_type = other DNA
gcgccaagcggtgtgttttagagctacctgaaggtaaagatcgggtcctcttttttttcagccaactccaattttttttgaggacccgatctttaccttcagg

SEQ ID NO: 113 - DNA, 103 bp, synthetic construct
  source 1..103: mol_type = other DNA
cgaggtcacggcgtgttttagagctaccttcccgacttccgtaggagcgcttttttttcagccaactccaattttttttgcgctcctacggaagtcgggaagg

SEQ ID NO: 114 - DNA, 103 bp, synthetic construct
  source 1..103: mol_type = other DNA
ccgcgtggtccgtcgttttagagctaccttcccgacttccgtaggagcgcttttttttcagccaactccaattttttttgcgctcctacggaagtcgggaagg

SEQ ID NO: 115 - DNA, 103 bp, synthetic construct
  source 1..103: mol_type = other DNA
ggggcagcgtcccagttttagagctaccttcccgacttccgtaggagcgcttttttttcagccaactccaattttttttgcgctcctacggaagtcgggaagg

SEQ ID NO: 116 - DNA, 103 bp, synthetic construct
  source 1..103: mol_type = other DNA
ccagaccttctggcgttttagagctaccttcccgacttccgtaggagcgcttttttttcagccaactccaattttttttgcgctcctacggaagtcgggaagg

SEQ ID NO: 117 - DNA, 101 bp, synthetic construct
  source 1..101: mol_type = other DNA
cgaggtcacggcgtgttttagagctaccggtgctccgcataccaagccgttttttttcagccaactccaattttttttcggcttggtatgcggagcaccgg

SEQ ID NO: 118 - DNA, 101 bp, synthetic construct
  source 1..101: mol_type = other DNA
ccgcgtggtccgtcgttttagagctaccggtgctccgcataccaagccgttttttttcagccaactccaattttttttcggcttggtatgcggagcaccgg

SEQ ID NO: 119 - DNA, 101 bp, synthetic construct
  source 1..101: mol_type = other DNA
ggggcagcgtcccagttttagagctaccggtgctccgcataccaagccgttttttttcagccaactccaattttttttcggcttggtatgcggagcaccgg

SEQ ID NO: 120 - DNA, 101 bp, synthetic construct
  source 1..101: mol_type = other DNA
ccagaccttctggcgttttagagctaccggtgctccgcataccaagccgttttttttcagccaactccaattttttttcggcttggtatgcggagcaccgg

SEQ ID NO: 121 - DNA, 103 bp, synthetic construct
  source 1..103: mol_type = other DNA
cgaggtcacggcgtgttttagagctacctgaaggtaaagatcgggtcctcttttttttcagccaactccaattttttttgaggacccgatctttaccttcagg

SEQ ID NO: 122 - DNA, 103 bp, synthetic construct
  source 1..103: mol_type = other DNA
ccgcgtggtccgtcgttttagagctacctgaaggtaaagatcgggtcctcttttttttcagccaactccaattttttttgaggacccgatctttaccttcagg

SEQ ID NO: 123 - DNA, 103 bp, synthetic construct
  source 1..103: mol_type = other DNA
ggggcagcgtcccagttttagagctacctgaaggtaaagatcgggtcctcttttttttcagccaactccaattttttttgaggacccgatctttaccttcagg

SEQ ID NO: 124 - DNA, 103 bp, synthetic construct
  source 1..103: mol_type = other DNA
ccagaccttctggcgttttagagctacctgaaggtaaagatcgggtcctcttttttttcagccaactccaattttttttgaggacccgatctttaccttcagg

SEQ ID NO: 125 - DNA, 103 bp, synthetic construct
  source 1..103: mol_type = other DNA
tatgcagccttaatgttttagagctaccttcccgacttccgtaggagcgcttttttttcagccaactccaattttttttgcgctcctacggaagtcgggaagg

SEQ ID NO: 126 - DNA, 103 bp, synthetic construct
  source 1..103: mol_type = other DNA
ctgccgcctggccagttttagagctaccttcccgacttccgtaggagcgcttttttttcagccaactccaattttttttgcgctcctacggaagtcgggaagg

SEQ ID NO: 127 - DNA, 103 bp, synthetic construct
  source 1..103: mol_type = other DNA
aaagtgacccgagagttttagagctaccttcccgacttccgtaggagcgcttttttttcagccaactccaattttttttgcgctcctacggaagtcgggaagg

SEQ ID NO: 128 - DNA, 103 bp, synthetic construct
  source 1..103: mol_type = other DNA
gctgcggatgccccgttttagagctaccttcccgacttccgtaggagcgcttttttttcagccaactccaattttttttgcgctcctacggaagtcgggaagg

SEQ ID NO: 129 - DNA, 101 bp, synthetic construct
  source 1..101: mol_type = other DNA
tatgcagccttaatgttttagagctaccggtgctccgcataccaagccgttttttttcagccaactccaattttttttcggcttggtatgcggagcaccgg

SEQ ID NO: 130 - DNA, 101 bp, synthetic construct
  source 1..101: mol_type = other DNA
ctgccgcctggccagttttagagctaccggtgctccgcataccaagccgttttttttcagccaactccaattttttttcggcttggtatgcggagcaccgg

SEQ ID NO: 131 - DNA, 101 bp, synthetic construct
  source 1..101: mol_type = other DNA
aaagtgacccgagagttttagagctaccggtgctccgcataccaagccgttttttttcagccaactccaattttttttcggcttggtatgcggagcaccgg

SEQ ID NO: 132 - DNA, 101 bp, synthetic construct
  source 1..101: mol_type = other DNA
gctgcggatgccccgttttagagctaccggtgctccgcataccaagccgttttttttcagccaactccaattttttttcggcttggtatgcggagcaccgg

SEQ ID NO: 133 - DNA, 103 bp, synthetic construct
  source 1..103: mol_type = other DNA
tatgcagccttaatgttttagagctacctgaaggtaaagatcgggtcctcttttttttcagccaactccaattttttttgaggacccgatctttaccttcagg

SEQ ID NO: 134 - DNA, 103 bp, synthetic construct
  source 1..103: mol_type = other DNA
ctgccgcctggccagttttagagctacctgaaggtaaagatcgggtcctcttttttttcagccaactccaattttttttgaggacccgatctttaccttcagg

SEQ ID NO: 135 - DNA, 103 bp, synthetic construct
  source 1..103: mol_type = other DNA
aaagtgacccgagagttttagagctacctgaaggtaaagatcgggtcctcttttttttcagccaactccaattttttttgaggacccgatctttaccttcagg

SEQ ID NO: 136 - DNA, 103 bp, synthetic construct
  source 1..103: mol_type = other DNA
gctgcggatgccccgttttagagctacctgaaggtaaagatcgggtcctcttttttttcagccaactccaattttttttgaggacccgatctttaccttcagg

SEQ ID NO: 137 - DNA, 103 bp, synthetic construct
  source 1..103: mol_type = other DNA
gggtagaccgtcccgttttagagctaccttcccgacttccgtaggagcgcttttttttcagccaactccaattttttttgcgctcctacggaagtcgggaagg

SEQ ID NO: 138 - DNA, 103 bp, synthetic construct
  source 1..103: mol_type = other DNA
cgagtgcggggatcgttttagagctaccttcccgacttccgtaggagcgcttttttttcagccaactccaattttttttgcgctcctacggaagtcgggaagg

SEQ ID NO: 139 - DNA, 103 bp, synthetic construct
  source 1..103: mol_type = other DNA
gcaccaccagagatgttttagagctaccttcccgacttccgtaggagcgcttttttttcagccaactccaattttttttgcgctcctacggaagtcgggaagg

SEQ ID NO: 140 - DNA, 103 bp, synthetic construct
  source 1..103: mol_type = other DNA
caaccttctctcctgttttagagctaccttcccgacttccgtaggagcgcttttttttcagccaactccaattttttttgcgctcctacggaagtcgggaagg

SEQ ID NO: 141 - DNA, 101 bp, synthetic construct
  source 1..101: mol_type = other DNA
gggtagaccgtcccgttttagagctaccggtgctccgcataccaagccgttttttttcagccaactccaattttttttcggcttggtatgcggagcaccgg

SEQ ID NO: 142 - DNA, 101 bp, synthetic construct
  source 1..101: mol_type = other DNA
cgagtgcggggatcgttttagagctaccggtgctccgcataccaagccgttttttttcagccaactccaattttttttcggcttggtatgcggagcaccgg

SEQ ID NO: 143 - DNA, 101 bp, synthetic construct
  source 1..101: mol_type = other DNA
gcaccaccagagatgttttagagctaccggtgctccgcataccaagccgttttttttcagccaactccaattttttttcggcttggtatgcggagcaccgg

SEQ ID NO: 144 - DNA, 101 bp, synthetic construct
  source 1..101: mol_type = other DNA
caaccttctctcctgttttagagctaccggtgctccgcataccaagccgttttttttcagccaactccaattttttttcggcttggtatgcggagcaccgg

SEQ ID NO: 145 - DNA, 103 bp, synthetic construct
  source 1..103: mol_type = other DNA
gggtagaccgtcccgttttagagctacctgaaggtaaagatcgggtcctcttttttttcagccaactccaattttttttgaggacccgatctttaccttcagg

SEQ ID NO: 146 - DNA, 103 bp, synthetic construct
  source 1..103: mol_type = other DNA
cgagtgcggggatcgttttagagctacctgaaggtaaagatcgggtcctcttttttttcagccaactccaattttttttgaggacccgatctttaccttcagg

SEQ ID NO: 147 - DNA, 103 bp, synthetic construct
  source 1..103: mol_type = other DNA
gcaccaccagagatgttttagagctacctgaaggtaaagatcgggtcctcttttttttcagccaactccaattttttttgaggacccgatctttaccttcagg

SEQ ID NO: 148 - DNA, 103 bp, synthetic construct
  source 1..103: mol_type = other DNA
caaccttctctcctgttttagagctacctgaaggtaaagatcgggtcctcttttttttcagccaactccaattttttttgaggacccgatctttaccttcagg

SEQ ID NO: 149 - DNA, 103 bp, synthetic construct
  source 1..103: mol_type = other DNA
tagggtgacttgaagttttagagctaccttcccgacttccgtaggagcgcttttttttcagccaactccaattttttttgcgctcctacggaagtcgggaagg

SEQ ID NO: 150 - DNA, 103 bp, synthetic construct
  source 1..103: mol_type = other DNA
acgaggtcgagccggttttagagctaccttcccgacttccgtaggagcgcttttttttcagccaactccaattttttttgcgctcctacggaagtcgggaagg

SEQ ID NO: 151 - DNA, 103 bp, synthetic construct
  source 1..103: mol_type = other DNA
ggcccattcatcaagttttagagctaccttcccgacttccgtaggagcgcttttttttcagccaactccaattttttttgcgctcctacggaagtcgggaagg

SEQ ID NO: 152 - DNA, 103 bp, synthetic construct
  source 1..103: mol_type = other DNA
gctcccttgcgcccgttttagagctaccttcccgacttccgtaggagcgcttttttttcagccaactccaattttttttgcgctcctacggaagtcgggaagg

SEQ ID NO: 153 - DNA, 101 bp, synthetic construct
  source 1..101: mol_type = other DNA
tagggtgacttgaagttttagagctaccggtgctccgcataccaagccgttttttttcagccaactccaattttttttcggcttggtatgcggagcaccgg

SEQ ID NO: 154 - DNA, 101 bp, synthetic construct
  source 1..101: mol_type = other DNA
acgaggtcgagccggttttagagctaccggtgctccgcataccaagccgttttttttcagccaactccaattttttttcggcttggtatgcggagcaccgg

SEQ ID NO: 155 - DNA, 101 bp, synthetic construct
  source 1..101: mol_type = other DNA
ggcccattcatcaagttttagagctaccggtgctccgcataccaagccgttttttttcagccaactccaattttttttcggcttggtatgcggagcaccgg

SEQ ID NO: 156 - DNA, 101 bp, synthetic construct
  source 1..101: mol_type = other DNA
gctcccttgcgcccgttttagagctaccggtgctccgcataccaagccgttttttttcagccaactccaattttttttcggcttggtatgcggagcaccgg

SEQ ID NO: 157 - DNA, 103 bp, synthetic construct
  source 1..103: mol_type = other DNA
tagggtgacttgaagttttagagctacctgaaggtaaagatcgggtcctcttttttttcagccaactccaattttttttgaggacccgatctttaccttcagg

SEQ ID NO: 158 - DNA, 103 bp, synthetic construct
  source 1..103: mol_type = other DNA
acgaggtcgagccggttttagagctacctgaaggtaaagatcgggtcctcttttttttcagccaactccaattttttttgaggacccgatctttaccttcagg

SEQ ID NO: 159 - DNA, 103 bp, synthetic construct
  source 1..103: mol_type = other DNA
ggcccattcatcaagttttagagctacctgaaggtaaagatcgggtcctcttttttttcagccaactccaattttttttgaggacccgatctttaccttcagg

SEQ ID NO: 160 - DNA, 103 bp, synthetic construct
  source 1..103: mol_type = other DNA
gctcccttgcgcccgttttagagctacctgaaggtaaagatcgggtcctcttttttttcagccaactccaattttttttgaggacccgatctttaccttcagg

SEQ ID NO: 161 - DNA, 101 bp, synthetic construct
  source 1..101: mol_type = other DNA
gctttcacggaagggttttagagctatagctaccgatgtcgagtgtaggttttttttcagccaactccaattttttttcctacactcgacatcggtagcta

SEQ ID NO: 162 - DNA, 101 bp, synthetic construct
  source 1..101: mol_type = other DNA
gctttcacggaagggttttagagctaattactcgaacgttccgccagggttttttttcagccaactccaattttttttccctggcggaacgttcgagtaat

SEQ ID NO: 163 - DNA, 101 bp, synthetic construct
  source 1..101: mol_type = other DNA
gctttcacggaagggttttagagctagcgcacgaccactatcgtgtaggttttttttcagccaactccaattttttttcctacacgatagtggtcgtgcgc

SEQ ID NO: 164 - DNA, 101 bp, synthetic construct
  source 1..101: mol_type = other DNA
gctttcacggaagggttttagagctaactcgttcgatagagagttcgggttttttttcagccaactccaattttttttcccgaactctctatcgaacgagt

SEQ ID NO: 165 - DNA, 101 bp, synthetic construct
  source 1..101: mol_type = other DNA
gctttcacggaagggttttagagctacctccgtgtcgatcgtgccatggttttttttcagccaactccaattttttttccatggcacgatcgacacggagg

SEQ ID NO: 166 - DNA, 101 bp, synthetic construct
  source 1..101: mol_type = other DNA
gctttcacggaagggttttagagctagctcagtcgcgaatgagctttggttttttttcagccaactccaattttttttccaaagctcattcgcgactgagc

SEQ ID NO: 167 - DNA, 101 bp, synthetic construct
  source 1..101: mol_type = other DNA
gctttcacggaagggttttagagctatagctcccgtccgtagacgtcggttttttttcagccaactccaattttttttccgacgtctacggacgggagcta

SEQ ID NO: 168 - DNA, 101 bp, synthetic construct
  source 1..101: mol_type = other DNA
gctttcacggaagggttttagagctatgcgtcgtctactacctctcgggttttttttcagccaactccaattttttttcccgagaggtagtagacgacgca

SEQ ID NO: 169 - DNA, 101 bp, synthetic construct
  source 1..101: mol_type = other DNA
gctttcacggaagggttttagagctatgctacgcatacgtgacgacgggttttttttcagccaactccaattttttttcccgtcgtcacgtatgcgtagca

SEQ ID NO: 170 - DNA, 101 bp, synthetic construct
  source 1..101: mol_type = other DNA
ctgcgcatcgggtggttttagagctatagctaccgatgtcgagtgtaggttttttttcagccaactccaattttttttcctacactcgacatcggtagcta

SEQ ID NO: 171 - DNA, 101 bp, synthetic construct
  source 1..101: mol_type = other DNA
ctgcgcatcgggtggttttagagctaattactcgaacgttccgccagggttttttttcagccaactccaattttttttccctggcggaacgttcgagtaat

SEQ ID NO: 172 - DNA, 101 bp, synthetic construct
  source 1..101: mol_type = other DNA
ctgcgcatcgggtggttttagagctagcgcacgaccactatcgtgtaggttttttttcagccaactccaattttttttcctacacgatagtggtcgtgcgc

SEQ ID NO: 173 - DNA, 101 bp, synthetic construct
  source 1..101: mol_type = other DNA
ctgcgcatcgggtggttttagagctaactcgttcgatagagagttcgggttttttttcagccaactccaattttttttcccgaactctctatcgaacgagt

SEQ ID NO: 174 - DNA, 101 bp, synthetic construct
  source 1..101: mol_type = other DNA
ctgcgcatcgggtggttttagagctacctccgtgtcgatcgtgccatggttttttttcagccaactccaattttttttccatggcacgatcgacacggagg

SEQ ID NO: 175 - DNA, 101 bp, synthetic construct
  source 1..101: mol_type = other DNA
ctgcgcatcgggtggttttagagctagctcagtcgcgaatgagctttggttttttttcagccaactccaattttttttccaaagctcattcgcgactgagc

SEQ ID NO: 176 - DNA, 101 bp, synthetic construct
  source 1..101: mol_type = other DNA
ctgcgcatcgggtggttttagagctatagctcccgtccgtagacgtcggttttttttcagccaactccaattttttttccgacgtctacggacgggagcta

SEQ ID NO: 177 - DNA, 101 bp, synthetic construct
  source 1..101: mol_type = other DNA
ctgcgcatcgggtggttttagagctatgcgtcgtctactacctctcgggttttttttcagccaactccaattttttttcccgagaggtagtagacgacgca

SEQ ID NO: 178 - DNA, 101 bp, synthetic construct
  source 1..101: mol_type = other DNA
ctgcgcatcgggtggttttagagctatgctacgcatacgtgacgacgggttttttttcagccaactccaattttttttcccgtcgtcacgtatgcgtagca

SEQ ID NO: 179 - DNA, 101 bp, synthetic construct
  source 1..101: mol_type = other DNA
ccgcacttccgcgagttttagagctatagctaccgatgtcgagtgtaggttttttttcagccaactccaattttttttcctacactcgacatcggtagcta

SEQ ID NO: 180 - DNA, 101 bp, synthetic construct
  source 1..101: mol_type = other DNA
ccgcacttccgcgagttttagagctaattactcgaacgttccgccagggttttttttcagccaactccaattttttttccctggcggaacgttcgagtaat

SEQ ID NO: 181 - DNA, 101 bp, synthetic construct
  source 1..101: mol_type = other DNA
ccgcacttccgcgagttttagagctagcgcacgaccactatcgtgtaggttttttttcagccaactccaattttttttcctacacgatagtggtcgtgcgc

SEQ ID NO: 182 - DNA, 101 bp, synthetic construct
  source 1..101: mol_type = other DNA
ccgcacttccgcgagttttagagctaactcgttcgatagagagttcgggttttttttcagccaactccaattttttttcccgaactctctatcgaacgagt

SEQ ID NO: 183 - DNA, 101 bp, synthetic construct
  source 1..101: mol_type = other DNA
ccgcacttccgcgagttttagagctacctccgtgtcgatcgtgccatggttttttttcagccaactccaattttttttccatggcacgatcgacacggagg

SEQ ID NO: 184 - DNA, 101 bp, synthetic construct
  source 1..101: mol_type = other DNA
ccgcacttccgcgagttttagagctagctcagtcgcgaatgagctttggttttttttcagccaactccaattttttttccaaagctcattcgcgactgagc

SEQ ID NO: 185 - DNA, 101 bp, synthetic construct
  source 1..101: mol_type = other DNA
ccgcacttccgcgagttttagagctatagctcccgtccgtagacgtcggttttttttcagccaactccaattttttttccgacgtctacggacgggagcta

SEQ ID NO: 186 - DNA, 101 bp, synthetic construct
  source 1..101: mol_type = other DNA
ccgcacttccgcgagttttagagctatgcgtcgtctactacctctcgggttttttttcagccaactccaattttttttcccgagaggtagtagacgacgca

SEQ ID NO: 187 - DNA, 101 bp, synthetic construct
  source 1..101: mol_type = other DNA
ccgcacttccgcgagttttagagctatgctacgcatacgtgacgacgggttttttttcagccaactccaattttttttcccgtcgtcacgtatgcgtagca

SEQ ID NO: 188 - DNA, 101 bp, synthetic construct
  source 1..101: mol_type = other DNA
gcgccaagcggtgtgttttagagctatagctaccgatgtcgagtgtaggttttttttcagccaactccaattttttttcctacactcgacatcggtagcta

SEQ ID NO: 189 - DNA, 101 bp, synthetic construct
  source 1..101: mol_type = other DNA
gcgccaagcggtgtgttttagagctaattactcgaacgttccgccagggttttttttcagccaactccaattttttttccctggcggaacgttcgagtaat

SEQ ID NO: 190 - DNA, 101 bp, synthetic construct
  source 1..101: mol_type = other DNA
gcgccaagcggtgtgttttagagctagcgcacgaccactatcgtgtaggttttttttcagccaactccaattttttttcctacacgatagtggtcgtgcgc

SEQ ID NO: 191 - DNA, 101 bp, synthetic construct
  source 1..101: mol_type = other DNA
gcgccaagcggtgtgttttagagctaactcgttcgatagagagttcgggttttttttcagccaactccaattttttttcccgaactctctatcgaacgagt

SEQ ID NO: 192 - DNA, 101 bp, synthetic construct
  source 1..101: mol_type = other DNA
gcgccaagcggtgtgttttagagctacctccgtgtcgatcgtgccatggttttttttcagccaactccaattttttttccatggcacgatcgacacggagg

SEQ ID NO: 193 - DNA, 101 bp, synthetic construct
  source 1..101: mol_type = other DNA
gcgccaagcggtgtgttttagagctagctcagtcgcgaatgagctttggttttttttcagccaactccaattttttttccaaagctcattcgcgactgagc

SEQ ID NO: 194 - DNA, 101 bp, synthetic construct
  source 1..101: mol_type = other DNA
gcgccaagcggtgtgttttagagctatagctcccgtccgtagacgtcggttttttttcagccaactccaattttttttccgacgtctacggacgggagcta

SEQ ID NO: 195 - DNA, 101 bp, synthetic construct
  source 1..101: mol_type = other DNA
gcgccaagcggtgtgttttagagctatgcgtcgtctactacctctcgggttttttttcagccaactccaattttttttcccgagaggtagtagacgacgca

SEQ ID NO: 196 - DNA, 101 bp, synthetic construct
  source 1..101: mol_type = other DNA
gcgccaagcggtgtgttttagagctatgctacgcatacgtgacgacgggttttttttcagccaactccaattttttttcccgtcgtcacgtatgcgtagca

SEQ ID NO: 197 - DNA, 101 bp, synthetic construct
  source 1..101: mol_type = other DNA
cgaggtcacggcgtgttttagagctatagctaccgatgtcgagtgtaggttttttttcagccaactccaattttttttcctacactcgacatcggtagcta

SEQ ID NO: 198 - DNA, 101 bp, synthetic construct
  source 1..101: mol_type = other DNA
cgaggtcacggcgtgttttagagctaattactcgaacgttccgccagggttttttttcagccaactccaattttttttccctggcggaacgttcgagtaat

SEQ ID NO: 199 - DNA, 101 bp, synthetic construct
  source 1..101: mol_type = other DNA
cgaggtcacggcgtgttttagagctagcgcacgaccactatcgtgtaggttttttttcagccaactccaattttttttcctacacgatagtggtcgtgcgc

SEQ ID NO: 200 - DNA, 101 bp, synthetic construct
  source 1..101: mol_type = other DNA
cgaggtcacggcgtgttttagagctaactcgttcgatagagagttcgggttttttttcagccaactccaattttttttcccgaactctctatcgaacgagt

SEQ ID NO: 201 - DNA, 101 bp, synthetic construct
  source 1..101: mol_type = other DNA
cgaggtcacggcgtgttttagagctacctccgtgtcgatcgtgccatggttttttttcagccaactccaattttttttccatggcacgatcgacacggagg

SEQ ID NO: 202 - DNA, 101 bp, synthetic construct
  source 1..101: mol_type = other DNA
cgaggtcacggcgtgttttagagctagctcagtcgcgaatgagctttggttttttttcagccaactccaattttttttccaaagctcattcgcgactgagc

SEQ ID NO: 203 - DNA, 101 bp, synthetic construct
  source 1..101: mol_type = other DNA
cgaggtcacggcgtgttttagagctatagctcccgtccgtagacgtcggttttttttcagccaactccaattttttttccgacgtctacggacgggagcta

SEQ ID NO: 204 - DNA, 101 bp, synthetic construct
  source 1..101: mol_type = other DNA
cgaggtcacggcgtgttttagagctatgcgtcgtctactacctctcgggttttttttcagccaactccaattttttttcccgagaggtagtagacgacgca

SEQ ID NO: 205 - DNA, 101 bp, synthetic construct
  source 1..101: mol_type = other DNA
cgaggtcacggcgtgttttagagctatgctacgcatacgtgacgacgggttttttttcagccaactccaattttttttcccgtcgtcacgtatgcgtagca

SEQ ID NO: 206 - DNA, 101 bp, synthetic construct
  source 1..101: mol_type = other DNA
ccgcgtggtccgtcgttttagagctatagctaccgatgtcgagtgtaggttttttttcagccaactccaattttttttcctacactcgacatcggtagcta

SEQ ID NO: 207 - DNA, 101 bp, synthetic construct
  source 1..101: mol_type = other DNA
ccgcgtggtccgtcgttttagagctaattactcgaacgttccgccagggttttttttcagccaactccaattttttttccctggcggaacgttcgagtaat

SEQ ID NO: 208 - DNA, 101 bp, synthetic construct
  source 1..101: mol_type = other DNA
ccgcgtggtccgtcgttttagagctagcgcacgaccactatcgtgtaggttttttttcagccaactccaattttttttcctacacgatagtggtcgtgcgc

SEQ ID NO: 209 - DNA, 101 bp, synthetic construct
  source 1..101: mol_type = other DNA
ccgcgtggtccgtcgttttagagctaactcgttcgatagagagttcgggttttttttcagccaactccaattttttttcccgaactctctatcgaacgagt

SEQ ID NO: 210 - DNA, 101 bp, synthetic construct
  source 1..101: mol_type = other DNA
ccgcgtggtccgtcgttttagagctacctccgtgtcgatcgtgccatggttttttttcagccaactccaattttttttccatggcacgatcgacacggagg

SEQ ID NO: 211 - DNA, 101 bp, synthetic construct
  source 1..101: mol_type = other DNA
ccgcgtggtccgtcgttttagagctagctcagtcgcgaatgagctttggttttttttcagccaactccaattttttttccaaagctcattcgcgactgagc

SEQ ID NO: 212 - DNA, 101 bp, synthetic construct
  source 1..101: mol_type = other DNA
ccgcgtggtccgtcgttttagagctatagctcccgtccgtagacgtcggttttttttcagccaactccaattttttttccgacgtctacggacgggagcta

SEQ ID NO: 213 - DNA, 101 bp, synthetic construct
  source 1..101: mol_type = other DNA
ccgcgtggtccgtcgttttagagctatgcgtcgtctactacctctcgggttttttttcagccaactccaattttttttcccgagaggtagtagacgacgca

SEQ ID NO: 214 - DNA, 101 bp, synthetic construct
  source 1..101: mol_type = other DNA
ccgcgtggtccgtcgttttagagctatgctacgcatacgtgacgacgggttttttttcagccaactccaattttttttcccgtcgtcacgtatgcgtagca

SEQ ID NO: 215 - DNA, 101 bp, synthetic construct
  source 1..101: mol_type = other DNA
ggggcagcgtcccagttttagagctatagctaccgatgtcgagtgtaggttttttttcagccaactccaattttttttcctacactcgacatcggtagcta

SEQ ID NO: 216 - DNA, 101 bp, synthetic construct
  source 1..101: mol_type = other DNA
ggggcagcgtcccagttttagagctaattactcgaacgttccgccagggttttttttcagccaactccaattttttttccctggcggaacgttcgagtaat

SEQ ID NO: 217 - DNA, 101 bp, synthetic construct
  source 1..101: mol_type = other DNA
ggggcagcgtcccagttttagagctagcgcacgaccactatcgtgtaggttttttttcagccaactccaattttttttcctacacgatagtggtcgtgcgc

SEQ ID NO: 218 - DNA, 101 bp, synthetic construct
  source 1..101: mol_type = other DNA
ggggcagcgtcccagttttagagctaactcgttcgatagagagttcgggttttttttcagccaactccaattttttttcccgaactctctatcgaacgagt

SEQ ID NO: 219 - DNA, 101 bp, synthetic construct
  source 1..101: mol_type = other DNA
ggggcagcgtcccagttttagagctacctccgtgtcgatcgtgccatggttttttttcagccaactccaattttttttccatggcacgatcgacacggagg

SEQ ID NO: 220 - DNA, 101 bp, synthetic construct
  source 1..101: mol_type = other DNA
ggggcagcgtcccagttttagagctagctcagtcgcgaatgagctttggttttttttcagccaactccaattttttttccaaagctcattcgcgactgagc

SEQ ID NO: 221 - DNA, 101 bp, synthetic construct
  source 1..101: mol_type = other DNA
ggggcagcgtcccagttttagagctatagctcccgtccgtagacgtcggttttttttcagccaactccaattttttttccgacgtctacggacgggagcta

SEQ ID NO: 222 - DNA, 101 bp, synthetic construct
  source 1..101: mol_type = other DNA
ggggcagcgtcccagttttagagctatgcgtcgtctactacctctcgggttttttttcagccaactccaattttttttcccgagaggtagtagacgacgca

SEQ ID NO: 223 - DNA, 101 bp, synthetic construct
  source 1..101: mol_type = other DNA
ggggcagcgtcccagttttagagctatgctacgcatacgtgacgacgggttttttttcagccaactccaattttttttcccgtcgtcacgtatgcgtagca

SEQ ID NO: 224 - DNA, 101 bp, synthetic construct
  source 1..101: mol_type = other DNA
ccagaccttctggcgttttagagctatagctaccgatgtcgagtgtaggttttttttcagccaactccaattttttttcctacactcgacatcggtagcta

SEQ ID NO: 225 - DNA, 101 bp, synthetic construct
  source 1..101: mol_type = other DNA
ccagaccttctggcgttttagagctaattactcgaacgttccgccagggttttttttcagccaactccaattttttttccctggcggaacgttcgagtaat

SEQ ID NO: 226 - DNA, 101 bp, synthetic construct
  source 1..101: mol_type = other DNA
ccagaccttctggcgttttagagctagcgcacgaccactatcgtgtaggttttttttcagccaactccaattttttttcctacacgatagtggtcgtgcgc

SEQ ID NO: 227 - DNA, 101 bp, synthetic construct
  source 1..101: mol_type = other DNA
ccagaccttctggcgttttagagctaactcgttcgatagagagttcgggttttttttcagccaactccaattttttttcccgaactctctatcgaacgagt

SEQ ID NO: 228 - DNA, 101 bp, synthetic construct
  source 1..101: mol_type = other DNA
ccagaccttctggcgttttagagctacctccgtgtcgatcgtgccatggttttttttcagccaactccaattttttttccatggcacgatcgacacggagg

SEQ ID NO: 229 - DNA, 101 bp, synthetic construct
  source 1..101: mol_type = other DNA
ccagaccttctggcgttttagagctagctcagtcgcgaatgagctttggttttttttcagccaactccaattttttttccaaagctcattcgcgactgagc

SEQ ID NO: 230 - DNA, 101 bp, synthetic construct
  source 1..101: mol_type = other DNA
ccagaccttctggcgttttagagctatagctcccgtccgtagacgtcggttttttttcagccaactccaattttttttccgacgtctacggacgggagcta

SEQ ID NO: 231 - DNA, 101 bp, synthetic construct
  source 1..101: mol_type = other DNA
ccagaccttctggcgttttagagctatgcgtcgtctactacctctcgggttttttttcagccaactccaattttttttcccgagaggtagtagacgacgca

SEQ ID NO: 232 - DNA, 101 bp, synthetic construct
  source 1..101: mol_type = other DNA
ccagaccttctggcgttttagagctatgctacgcatacgtgacgacgggttttttttcagccaactccaattttttttcccgtcgtcacgtatgcgtagca

SEQ ID NO: 233 - DNA, 101 bp, synthetic construct
  source 1..101: mol_type = other DNA
tatgcagccttaatgttttagagctatagctaccgatgtcgagtgtaggttttttttcagccaactccaattttttttcctacactcgacatcggtagcta

SEQ ID NO: 234 - DNA, 101 bp, synthetic construct
  source 1..101: mol_type = other DNA
tatgcagccttaatgttttagagctaattactcgaacgttccgccagggttttttttcagccaactccaattttttttccctggcggaacgttcgagtaat

SEQ ID NO: 235 - DNA, 101 bp, synthetic construct
  source 1..101: mol_type = other DNA
tatgcagccttaatgttttagagctagcgcacgaccactatcgtgtaggttttttttcagccaactccaattttttttcctacacgatagtggtcgtgcgc

SEQ ID NO: 236 - DNA, 101 bp, synthetic construct
  source 1..101: mol_type = other DNA
tatgcagccttaatgttttagagctaactcgttcgatagagagttcgggttttttttcagccaactccaattttttttcccgaactctctatcgaacgagt

SEQ ID NO: 237 - DNA, 101 bp, synthetic construct
  source 1..101: mol_type = other DNA
tatgcagccttaatgttttagagctacctccgtgtcgatcgtgccatggttttttttcagccaactccaattttttttccatggcacgatcgacacggagg

SEQ ID NO: 238 - DNA, 101 bp, synthetic construct
  source 1..101: mol_type = other DNA
tatgcagccttaatgttttagagctagctcagtcgcgaatgagctttggttttttttcagccaactccaattttttttccaaagctcattcgcgactgagc

SEQ ID NO: 239 - DNA, 101 bp, synthetic construct
  source 1..101: mol_type = other DNA
tatgcagccttaatgttttagagctatagctcccgtccgtagacgtcggttttttttcagccaactccaattttttttccgacgtctacggacgggagcta

SEQ ID NO: 240 - DNA, 101 bp, synthetic construct
  source 1..101: mol_type = other DNA
tatgcagccttaatgttttagagctatgcgtcgtctactacctctcgggttttttttcagccaactccaattttttttcccgagaggtagtagacgacgca

SEQ ID NO: 241 - DNA, 101 bp, synthetic construct
  source 1..101: mol_type = other DNA
tatgcagccttaatgttttagagctatgctacgcatacgtgacgacgggttttttttcagccaactccaattttttttcccgtcgtcacgtatgcgtagca

SEQ ID NO: 242 - DNA, 101 bp, synthetic construct
  source 1..101: mol_type = other DNA
ctgccgcctggccagttttagagctatagctaccgatgtcgagtgtaggttttttttcagccaactccaattttttttcctacactcgacatcggtagcta

SEQ ID NO: 243 - DNA, 101 bp, synthetic construct
  source 1..101: mol_type = other DNA
ctgccgcctggccagttttagagctaattactcgaacgttccgccagggttttttttcagccaactccaattttttttccctggcggaacgttcgagtaat

SEQ ID NO: 244 - DNA, 101 bp, synthetic construct
  source 1..101: mol_type = other DNA
ctgccgcctggccagttttagagctagcgcacgaccactatcgtgtaggttttttttcagccaactccaattttttttcctacacgatagtggtcgtgcgc

SEQ ID NO: 245 - DNA, 101 bp, synthetic construct
  source 1..101: mol_type = other DNA
ctgccgcctggccagttttagagctaactcgttcgatagagagttcgggttttttttcagccaactccaattttttttcccgaactctctatcgaacgagt

SEQ ID NO: 246 - DNA, 101 bp, synthetic construct
  source 1..101: mol_type = other DNA
ctgccgcctggccagttttagagctacctccgtgtcgatcgtgccatggttttttttcagccaactccaattttttttccatggcacgatcgacacggagg

SEQ ID NO: 247 - DNA, 101 bp, synthetic construct
  source 1..101: mol_type = other DNA
ctgccgcctggccagttttagagctagctcagtcgcgaatgagctttggttttttttcagccaactccaattttttttccaaagctcattcgcgactgagc

SEQ ID NO: 248 - DNA, 101 bp, synthetic construct
  source 1..101: mol_type = other DNA
ctgccgcctggccagttttagagctatagctcccgtccgtagacgtcggttttttttcagccaactccaattttttttccgacgtctacggacgggagcta

SEQ ID NO: 249 - DNA, 101 bp, synthetic construct
  source 1..101: mol_type = other DNA
ctgccgcctggccagttttagagctatgcgtcgtctactacctctcgggttttttttcagccaactccaattttttttcccgagaggtagtagacgacgca

SEQ ID NO: 250 - DNA, 101 bp, synthetic construct
  source 1..101: mol_type = other DNA
ctgccgcctggccagttttagagctatgctacgcatacgtgacgacgggttttttttcagccaactccaattttttttcccgtcgtcacgtatgcgtagca

SEQ ID NO: 251 - DNA, 101 bp, synthetic construct
  source 1..101: mol_type = other DNA
aaagtgacccgagagttttagagctatagctaccgatgtcgagtgtaggttttttttcagccaactccaattttttttcctacactcgacatcggtagcta

SEQ ID NO: 252 - DNA, 101 bp, synthetic construct
  source 1..101: mol_type = other DNA
aaagtgacccgagagttttagagctaattactcgaacgttccgccagggttttttttcagccaactccaattttttttccctggcggaacgttcgagtaat

SEQ ID NO: 253 - DNA, 101 bp, synthetic construct
  source 1..101: mol_type = other DNA
aaagtgacccgagagttttagagctagcgcacgaccactatcgtgtaggttttttttcagccaactccaattttttttcctacacgatagtggtcgtgcgc

SEQ ID NO: 254 - DNA, 101 bp, synthetic construct
  source 1..101: mol_type = other DNA
aaagtgacccgagagttttagagctaactcgttcgatagagagttcgggttttttttcagccaactccaattttttttcccgaactctctatcgaacgagt

SEQ ID NO: 255 - DNA, 101 bp, synthetic construct
  source 1..101: mol_type = other DNA
aaagtgacccgagagttttagagctacctccgtgtcgatcgtgccatggttttttttcagccaactccaattttttttccatggcacgatcgacacggagg

SEQ ID NO: 256 - DNA, 101 bp, synthetic construct
  source 1..101: mol_type = other DNA
aaagtgacccgagagttttagagctagctcagtcgcgaatgagctttggttttttttcagccaactccaattttttttccaaagctcattcgcgactgagc

SEQ ID NO: 257 - DNA, 101 bp, synthetic construct
  source 1..101: mol_type = other DNA
aaagtgacccgagagttttagagctatagctcccgtccgtagacgtcggttttttttcagccaactccaattttttttccgacgtctacggacgggagcta

SEQ ID NO: 258 - DNA, 101 bp, synthetic construct
  source 1..101: mol_type = other DNA
aaagtgacccgagagttttagagctatgcgtcgtctactacctctcgggttttttttcagccaactccaattttttttcccgagaggtagtagacgacgca

SEQ ID NO: 259 - DNA, 101 bp, synthetic construct
  source 1..101: mol_type = other DNA
aaagtgacccgagagttttagagctatgctacgcatacgtgacgacgggttttttttcagccaactccaattttttttcccgtcgtcacgtatgcgtagca

SEQ ID NO: 260 - DNA, 101 bp, synthetic construct
  source 1..101: mol_type = other DNA
gctgcggatgccccgttttagagctatagctaccgatgtcgagtgtaggttttttttcagccaactccaattttttttcctacactcgacatcggtagcta

SEQ ID NO: 261 - DNA, 101 bp, synthetic construct
  source 1..101: mol_type = other DNA
gctgcggatgccccgttttagagctaattactcgaacgttccgccagggttttttttcagccaactccaattttttttccctggcggaacgttcgagtaat

SEQ ID NO: 262 - DNA, 101 bp, synthetic construct
  source 1..101: mol_type = other DNA
gctgcggatgccccgttttagagctagcgcacgaccactatcgtgtaggttttttttcagccaactccaattttttttcctacacgatagtggtcgtgcgc

SEQ ID NO: 263 - DNA, 101 bp, synthetic construct
  source 1..101: mol_type = other DNA
gctgcggatgccccgttttagagctaactcgttcgatagagagttcgggttttttttcagccaactccaattttttttcccgaactctctatcgaacgagt

SEQ ID NO: 264 - DNA, 101 bp, synthetic construct
  source 1..101: mol_type = other DNA
gctgcggatgccccgttttagagctacctccgtgtcgatcgtgccatggttttttttcagccaactccaattttttttccatggcacgatcgacacggagg

SEQ ID NO: 265 - DNA, 101 bp, synthetic construct
  source 1..101: mol_type = other DNA
gctgcggatgccccgttttagagctagctcagtcgcgaatgagctttggttttttttcagccaactccaattttttttccaaagctcattcgcgactgagc

SEQ ID NO: 266 - DNA, 101 bp, synthetic construct
  source 1..101: mol_type = other DNA
gctgcggatgccccgttttagagctatagctcccgtccgtagacgtcggttttttttcagccaactccaattttttttccgacgtctacggacgggagcta

SEQ ID NO: 267 - DNA, 101 bp, synthetic construct
  source 1..101: mol_type = other DNA
gctgcggatgccccgttttagagctatgcgtcgtctactacctctcgggttttttttcagccaactccaattttttttcccgagaggtagtagacgacgca

SEQ ID NO: 268 - DNA, 101 bp, synthetic construct
  source 1..101: mol_type = other DNA
gctgcggatgccccgttttagagctatgctacgcatacgtgacgacgggttttttttcagccaactccaattttttttcccgtcgtcacgtatgcgtagca

SEQ ID NO: 269 - DNA, 101 bp, synthetic construct
  source 1..101: mol_type = other DNA
gggtagaccgtcccgttttagagctatagctaccgatgtcgagtgtaggttttttttcagccaactccaattttttttcctacactcgacatcggtagcta

SEQ ID NO: 270 - DNA, 101 bp, synthetic construct
  source 1..101: mol_type = other DNA
gggtagaccgtcccgttttagagctaattactcgaacgttccgccagggttttttttcagccaactccaattttttttccctggcggaacgttcgagtaat

SEQ ID NO: 271 - DNA, 101 bp, synthetic construct
  source 1..101: mol_type = other DNA
gggtagaccgtcccgttttagagctagcgcacgaccactatcgtgtaggttttttttcagccaactccaattttttttcctacacgatagtggtcgtgcgc

SEQ ID NO: 272 - DNA, 101 bp, synthetic construct
  source 1..101: mol_type = other DNA
gggtagaccgtcccgttttagagctaactcgttcgatagagagttcgggttttttttcagccaactccaattttttttcccgaactctctatcgaacgagt

SEQ ID NO: 273 - DNA, 101 bp, synthetic construct
  source 1..101: mol_type = other DNA
gggtagaccgtcccgttttagagctacctccgtgtcgatcgtgccatggttttttttcagccaactccaattttttttccatggcacgatcgacacggagg

SEQ ID NO: 274 - DNA, 101 bp, synthetic construct
  source 1..101: mol_type = other DNA
gggtagaccgtcccgttttagagctagctcagtcgcgaatgagctttggttttttttcagccaactccaattttttttccaaagctcattcgcgactgagc

SEQ ID NO: 275 - DNA, 101 bp, synthetic construct
  source 1..101: mol_type = other DNA
gggtagaccgtcccgttttagagctatagctcccgtccgtagacgtcggttttttttcagccaactccaattttttttccgacgtctacggacgggagcta

SEQ ID NO: 276 - DNA, 101 bp, synthetic construct
  source 1..101: mol_type = other DNA
gggtagaccgtcccgttttagagctatgcgtcgtctactacctctcgggttttttttcagccaactccaattttttttcccgagaggtagtagacgacgca

SEQ ID NO: 277 - DNA, 101 bp, synthetic construct
  source 1..101: mol_type = other DNA
gggtagaccgtcccgttttagagctatgctacgcatacgtgacgacgggttttttttcagccaactccaattttttttcccgtcgtcacgtatgcgtagca

SEQ ID NO: 278 - DNA, 101 bp, synthetic construct
  source 1..101: mol_type = other DNA
cgagtgcggggatcgttttagagctatagctaccgatgtcgagtgtaggttttttttcagccaactccaattttttttcctacactcgacatcggtagcta

SEQ ID NO: 279 - DNA, 101 bp, synthetic construct
  source 1..101: mol_type = other DNA
cgagtgcggggatcgttttagagctaattactcgaacgttccgccagggttttttttcagccaactccaattttttttccctggcggaacgttcgagtaat

SEQ ID NO: 280 - DNA, 101 bp, synthetic construct
  source 1..101: mol_type = other DNA
cgagtgcggggatcgttttagagctagcgcacgaccactatcgtgtaggttttttttcagccaactccaattttttttcctacacgatagtggtcgtgcgc

SEQ ID NO: 281 - DNA, 101 bp, synthetic construct
  source 1..101: mol_type = other DNA
cgagtgcggggatcgttttagagctaactcgttcgatagagagttcgggttttttttcagccaactccaattttttttcccgaactctctatcgaacgagt

SEQ ID NO: 282 - DNA, 101 bp, synthetic construct
  source 1..101: mol_type = other DNA
cgagtgcggggatcgttttagagctacctccgtgtcgatcgtgccatggttttttttcagccaactccaattttttttccatggcacgatcgacacggagg

SEQ ID NO: 283 - DNA, 101 bp, synthetic construct
  source 1..101: mol_type = other DNA
cgagtgcggggatcgttttagagctagctcagtcgcgaatgagctttggttttttttcagccaactccaattttttttccaaagctcattcgcgactgagc

SEQ ID NO: 284 - DNA, 101 bp, synthetic construct
  source 1..101: mol_type = other DNA
cgagtgcggggatcgttttagagctatagctcccgtccgtagacgtcggttttttttcagccaactccaattttttttccgacgtctacggacgggagcta

SEQ ID NO: 285 - DNA, 101 bp, synthetic construct
  source 1..101: mol_type = other DNA
cgagtgcggggatcgttttagagctatgcgtcgtctactacctctcgggttttttttcagccaactccaattttttttcccgagaggtagtagacgacgca

SEQ ID NO: 286 - DNA, 101 bp, synthetic construct
  source 1..101: mol_type = other DNA
cgagtgcggggatcgttttagagctatgctacgcatacgtgacgacgggttttttttcagccaactccaattttttttcccgtcgtcacgtatgcgtagca

SEQ ID NO: 287 - DNA, 101 bp, synthetic construct
  source 1..101: mol_type = other DNA
gcaccaccagagatgttttagagctatagctaccgatgtcgagtgtaggttttttttcagccaactccaattttttttcctacactcgacatcggtagcta

SEQ ID NO: 288 - DNA, 101 bp, synthetic construct
  source 1..101: mol_type = other DNA
gcaccaccagagatgttttagagctaattactcgaacgttccgccagggttttttttcagccaactccaattttttttccctggcggaacgttcgagtaat

SEQ ID NO: 289 - DNA, 101 bp, synthetic construct
  source 1..101: mol_type = other DNA
gcaccaccagagatgttttagagctagcgcacgaccactatcgtgtaggttttttttcagccaactccaattttttttcctacacgatagtggtcgtgcgc

SEQ ID NO: 290 - DNA, 101 bp, synthetic construct
  source 1..101: mol_type = other DNA
gcaccaccagagatgttttagagctaactcgttcgatagagagttcgggttttttttcagccaactccaattttttttcccgaactctctatcgaacgagt

SEQ ID NO: 291 - DNA, 101 bp, synthetic construct
  source 1..101: mol_type = other DNA
gcaccaccagagatgttttagagctacctccgtgtcgatcgtgccatggttttttttcagccaactccaattttttttccatggcacgatcgacacggagg

SEQ ID NO: 292 - DNA, 101 bp, synthetic construct
  source 1..101: mol_type = other DNA
gcaccaccagagatgttttagagctagctcagtcgcgaatgagctttggttttttttcagccaactccaattttttttccaaagctcattcgcgactgagc

SEQ ID NO: 293 - DNA, 101 bp, synthetic construct
  source 1..101: mol_type = other DNA
gcaccaccagagatgttttagagctatagctcccgtccgtagacgtcggttttttttcagccaactccaattttttttccgacgtctacggacgggagcta

SEQ ID NO: 294 - DNA, 101 bp, synthetic construct
  source 1..101: mol_type = other DNA
gcaccaccagagatgttttagagctatgcgtcgtctactacctctcgggttttttttcagccaactccaattttttttcccgagaggtagtagacgacgca

SEQ ID NO: 295 - DNA, 101 bp, synthetic construct
  source 1..101: mol_type = other DNA
gcaccaccagagatgttttagagctatgctacgcatacgtgacgacgggttttttttcagccaactccaattttttttcccgtcgtcacgtatgcgtagca

SEQ ID NO: 296 - DNA, 101 bp, synthetic construct
  source 1..101: mol_type = other DNA
caaccttctctcctgttttagagctatagctaccgatgtcgagtgtaggttttttttcagccaactccaattttttttcctacactcgacatcggtagcta

SEQ ID NO: 297 - DNA, 101 bp, synthetic construct
  source 1..101: mol_type = other DNA
caaccttctctcctgttttagagctaattactcgaacgttccgccagggttttttttcagccaactccaattttttttccctggcggaacgttcgagtaat

SEQ ID NO: 298 - DNA, 101 bp, synthetic construct
  source 1..101: mol_type = other DNA
caaccttctctcctgttttagagctagcgcacgaccactatcgtgtaggttttttttcagccaactccaattttttttcctacacgatagtggtcgtgcgc

SEQ ID NO: 299 - DNA, 101 bp, synthetic construct
  source 1..101: mol_type = other DNA
caaccttctctcctgttttagagctaactcgttcgatagagagttcgggttttttttcagccaactccaattttttttcccgaactctctatcgaacgagt

SEQ ID NO: 300 - DNA, 101 bp, synthetic construct
  source 1..101: mol_type = other DNA
caaccttctctcctgttttagagctacctccgtgtcgatcgtgccatggttttttttcagccaactccaattttttttccatggcacgatcgacacggagg

SEQ ID NO: 301 - DNA, 101 bp, synthetic construct
  source 1..101: mol_type = other DNA
caaccttctctcctgttttagagctagctcagtcgcgaatgagctttggttttttttcagccaactccaattttttttccaaagctcattcgcgactgagc

SEQ ID NO: 302 - DNA, 101 bp, synthetic construct
  source 1..101: mol_type = other DNA
caaccttctctcctgttttagagctatagctcccgtccgtagacgtcggttttttttcagccaactccaattttttttccgacgtctacggacgggagcta

SEQ ID NO: 303 - DNA, 101 bp, synthetic construct
  source 1..101: mol_type = other DNA
caaccttctctcctgttttagagctatgcgtcgtctactacctctcgggttttttttcagccaactccaattttttttcccgagaggtagtagacgacgca

SEQ ID NO: 304 - DNA, 101 bp, synthetic construct
  source 1..101: mol_type = other DNA
caaccttctctcctgttttagagctatgctacgcatacgtgacgacgggttttttttcagccaactccaattttttttcccgtcgtcacgtatgcgtagca

SEQ ID NO: 305 - DNA, 101 bp, synthetic construct
  source 1..101: mol_type = other DNA
tagggtgacttgaagttttagagctatagctaccgatgtcgagtgtaggttttttttcagccaactccaattttttttcctacactcgacatcggtagcta

SEQ ID NO: 306 - DNA, 101 bp, synthetic construct
  source 1..101: mol_type = other DNA
tagggtgacttgaagttttagagctaattactcgaacgttccgccagggttttttttcagccaactccaattttttttccctggcggaacgttcgagtaat

SEQ ID NO: 307 - DNA, 101 bp, synthetic construct
  source 1..101: mol_type = other DNA
tagggtgacttgaagttttagagctagcgcacgaccactatcgtgtaggttttttttcagccaactccaattttttttcctacacgatagtggtcgtgcgc

SEQ ID NO: 308 - DNA, 101 bp, synthetic construct
  source 1..101: mol_type = other DNA
tagggtgacttgaagttttagagctaactcgttcgatagagagttcgggttttttttcagccaactccaattttttttcccgaactctctatcgaacgagt

SEQ ID NO: 309 - DNA, 101 bp, synthetic construct
  source 1..101: mol_type = other DNA
tagggtgacttgaagttttagagctacctccgtgtcgatcgtgccatggttttttttcagccaactccaattttttttccatggcacgatcgacacggagg

SEQ ID NO: 310-399 - skipped (90 entries)

SEQ ID NO: 400 - DNA, 101 bp, synthetic construct
  source 1..101: mol_type = other DNA
tagggtgacttgaagttttagagctagctcagtcgcgaatgagctttggttttttttcagccaactccaattttttttccaaagctcattcgcgactgagc

SEQ ID NO: 401 - DNA, 101 bp, synthetic construct
  source 1..101: mol_type = other DNA
tagggtgacttgaagttttagagctatagctcccgtccgtagacgtcggttttttttcagccaactccaattttttttccgacgtctacggacgggagcta

SEQ ID NO: 402 - DNA, 101 bp, synthetic construct
  source 1..101: mol_type = other DNA
tagggtgacttgaagttttagagctatgcgtcgtctactacctctcgggttttttttcagccaactccaattttttttcccgagaggtagtagacgacgca

SEQ ID NO: 403 - DNA, 101 bp, synthetic construct
  source 1..101: mol_type = other DNA
tagggtgacttgaagttttagagctatgctacgcatacgtgacgacgggttttttttcagccaactccaattttttttcccgtcgtcacgtatgcgtagca

SEQ ID NO: 404 - DNA, 101 bp, synthetic construct
  source 1..101: mol_type = other DNA
acgaggtcgagccggttttagagctatagctaccgatgtcgagtgtaggttttttttcagccaactccaattttttttcctacactcgacatcggtagcta

SEQ ID NO: 405 - DNA, 101 bp, synthetic construct
  source 1..101: mol_type = other DNA
acgaggtcgagccggttttagagctaattactcgaacgttccgccagggttttttttcagccaactccaattttttttccctggcggaacgttcgagtaat

SEQ ID NO: 406 - DNA, 101 bp, synthetic construct
  source 1..101: mol_type = other DNA
acgaggtcgagccggttttagagctagcgcacgaccactatcgtgtaggttttttttcagccaactccaattttttttcctacacgatagtggtcgtgcgc

SEQ ID NO: 407 - DNA, 101 bp, synthetic construct
  source 1..101: mol_type = other DNA
acgaggtcgagccggttttagagctaactcgttcgatagagagttcgggttttttttcagccaactccaattttttttcccgaactctctatcgaacgagt

SEQ ID NO: 408 - DNA, 101 bp, synthetic construct
  source 1..101: mol_type = other DNA
acgaggtcgagccggttttagagctacctccgtgtcgatcgtgccatggttttttttcagccaactccaattttttttccatggcacgatcgacacggagg

SEQ ID NO: 409 - DNA, 101 bp, synthetic construct
  source 1..101: mol_type = other DNA
acgaggtcgagccggttttagagctagctcagtcgcgaatgagctttggttttttttcagccaactccaattttttttccaaagctcattcgcgactgagc

SEQ ID NO: 410 - DNA, 101 bp, synthetic construct
  source 1..101: mol_type = other DNA
acgaggtcgagccggttttagagctatagctcccgtccgtagacgtcggttttttttcagccaactccaattttttttccgacgtctacggacgggagcta

SEQ ID NO: 411 - DNA, 101 bp, synthetic construct
  source 1..101: mol_type = other DNA
acgaggtcgagccggttttagagctatgcgtcgtctactacctctcgggttttttttcagccaactccaattttttttcccgagaggtagtagacgacgca

SEQ ID NO: 412 - DNA, 101 bp, synthetic construct
  source 1..101: mol_type = other DNA
acgaggtcgagccggttttagagctatgctacgcatacgtgacgacgggttttttttcagccaactccaattttttttcccgtcgtcacgtatgcgtagca

SEQ ID NO: 413 - DNA, 101 bp, synthetic construct
  source 1..101: mol_type = other DNA
ggcccattcatcaagttttagagctatagctaccgatgtcgagtgtaggttttttttcagccaactccaattttttttcctacactcgacatcggtagcta

SEQ ID NO: 414 - DNA, 101 bp, synthetic construct
  source 1..101: mol_type = other DNA
ggcccattcatcaagttttagagctaattactcgaacgttccgccagggttttttttcagccaactccaattttttttccctggcggaacgttcgagtaat

SEQ ID NO: 415 - DNA, 101 bp, synthetic construct
  source 1..101: mol_type = other DNA
ggcccattcatcaagttttagagctagcgcacgaccactatcgtgtaggttttttttcagccaactccaattttttttcctacacgatagtggtcgtgcgc

SEQ ID NO: 416 - DNA, 101 bp, synthetic construct
  source 1..101: mol_type = other DNA
ggcccattcatcaagttttagagctaactcgttcgatagagagttcgggttttttttcagccaactccaattttttttcccgaactctctatcgaacgagt

SEQ ID NO: 417 - DNA, 101 bp, synthetic construct
  source 1..101: mol_type = other DNA
ggcccattcatcaagttttagagctacctccgtgtcgatcgtgccatggttttttttcagccaactccaattttttttccatggcacgatcgacacggagg

SEQ ID NO: 418 - DNA, 101 bp, synthetic construct
  source 1..101: mol_type = other DNA
ggcccattcatcaagttttagagctagctcagtcgcgaatgagctttggttttttttcagccaactccaattttttttccaaagctcattcgcgactgagc

SEQ ID NO: 419 - DNA, 101 bp, synthetic construct
  source 1..101: mol_type = other DNA
ggcccattcatcaagttttagagctatagctcccgtccgtagacgtcggttttttttcagccaactccaattttttttccgacgtctacggacgggagcta

SEQ ID NO: 420 - DNA, 101 bp, synthetic construct
  source 1..101: mol_type = other DNA
ggcccattcatcaagttttagagctatgcgtcgtctactacctctcgggttttttttcagccaactccaattttttttcccgagaggtagtagacgacgca

SEQ ID NO: 421 - DNA, 101 bp, synthetic construct
  source 1..101: mol_type = other DNA
ggcccattcatcaagttttagagctatgctacgcatacgtgacgacgggttttttttcagccaactccaattttttttcccgtcgtcacgtatgcgtagca

SEQ ID NO: 422 - DNA, 101 bp, synthetic construct
  source 1..101: mol_type = other DNA
gctcccttgcgcccgttttagagctatagctaccgatgtcgagtgtaggttttttttcagccaactccaattttttttcctacactcgacatcggtagcta

SEQ ID NO: 423 - DNA, 101 bp, synthetic construct
  source 1..101: mol_type = other DNA
gctcccttgcgcccgttttagagctaattactcgaacgttccgccagggttttttttcagccaactccaattttttttccctggcggaacgttcgagtaat

SEQ ID NO: 424 - DNA, 101 bp, synthetic construct
  source 1..101: mol_type = other DNA
gctcccttgcgcccgttttagagctagcgcacgaccactatcgtgtaggttttttttcagccaactccaattttttttcctacacgatagtggtcgtgcgc

SEQ ID NO: 425 - DNA, 101 bp, synthetic construct
  source 1..101: mol_type = other DNA
gctcccttgcgcccgttttagagctaactcgttcgatagagagttcgggttttttttcagccaactccaattttttttcccgaactctctatcgaacgagt

SEQ ID NO: 426 - DNA, 101 bp, synthetic construct
  source 1..101: mol_type = other DNA
gctcccttgcgcccgttttagagctacctccgtgtcgatcgtgccatggttttttttcagccaactccaattttttttccatggcacgatcgacacggagg

SEQ ID NO: 427 - DNA, 101 bp, synthetic construct
  source 1..101: mol_type = other DNA
gctcccttgcgcccgttttagagctagctcagtcgcgaatgagctttggttttttttcagccaactccaattttttttccaaagctcattcgcgactgagc

SEQ ID NO: 428 - DNA, 101 bp, synthetic construct
  source 1..101: mol_type = other DNA
gctcccttgcgcccgttttagagctatagctcccgtccgtagacgtcggttttttttcagccaactccaattttttttccgacgtctacggacgggagcta

SEQ ID NO: 429 - DNA, 101 bp, synthetic construct
  source 1..101: mol_type = other DNA
gctcccttgcgcccgttttagagctatgcgtcgtctactacctctcgggttttttttcagccaactccaattttttttcccgagaggtagtagacgacgca

SEQ ID NO: 430 - DNA, 101 bp, synthetic construct
  source 1..101: mol_type = other DNA
gctcccttgcgcccgttttagagctatgctacgcatacgtgacgacgggttttttttcagccaactccaattttttttcccgtcgtcacgtatgcgtagca

SEQ ID NO: 431 - DNA, 23 bp, synthetic construct
  source 1..23: mol_type = other DNA
tagctaccgatgtcgagtgtagg

SEQ ID NO: 432 - DNA, 23 bp, synthetic construct
  source 1..23: mol_type = other DNA
attactcgaacgttccgccaggg

SEQ ID NO: 433 - DNA, 23 bp, synthetic construct
  source 1..23: mol_type = other DNA
gcgcacgaccactatcgtgtagg

SEQ ID NO: 434 - DNA, 23 bp, synthetic construct
  source 1..23: mol_type = other DNA
actcgttcgatagagagttcggg

SEQ ID NO: 435 - DNA, 23 bp, synthetic construct
  source 1..23: mol_type = other DNA
cctccgtgtcgatcgtgccatgg

SEQ ID NO: 436 - DNA, 23 bp, synthetic construct
  source 1..23: mol_type = other DNA
gctcagtcgcgaatgagctttgg

SEQ ID NO: 437 - DNA, 23 bp, synthetic construct
  source 1..23: mol_type = other DNA
tagctcccgtccgtagacgtcgg

SEQ ID NO: 438 - DNA, 23 bp, synthetic construct
  source 1..23: mol_type = other DNA
tgcgtcgtctactacctctcggg

SEQ ID NO: 439 - DNA, 23 bp, synthetic construct
  source 1..23: mol_type = other DNA
tgctacgcatacgtgacgacggg

SEQ ID NO: 440 - DNA, 23 bp, synthetic construct
  source 1..23: mol_type = other DNA
cctacactcgacatcggtagcta

SEQ ID NO: 441 - DNA, 23 bp, synthetic construct
  source 1..23: mol_type = other DNA
ccctggcggaacgttcgagtaat

SEQ ID NO: 442 - DNA, 23 bp, synthetic construct
  source 1..23: mol_type = other DNA
cctacacgatagtggtcgtgcgc

SEQ ID NO: 443 - DNA, 23 bp, synthetic construct
  source 1..23: mol_type = other DNA
cccgaactctctatcgaacgagt

SEQ ID NO: 444 - DNA, 23 bp, synthetic construct
  source 1..23: mol_type = other DNA
ccatggcacgatcgacacggagg

SEQ ID NO: 445 - DNA, 23 bp, synthetic construct
  source 1..23: mol_type = other DNA
ccaaagctcattcgcgactgagc

SEQ ID NO: 446 - DNA, 23 bp, synthetic construct
  source 1..23: mol_type = other DNA
ccgacgtctacggacgggagcta

SEQ ID NO: 447 - DNA, 23 bp, synthetic construct
  source 1..23: mol_type = other DNA
cccgagaggtagtagacgacgca

SEQ ID NO: 448 - DNA, 23 bp, synthetic construct
  source 1..23: mol_type = other DNA
cccgtcgtcacgtatgcgtagca

SEQ ID NO: 449 - DNA, 102 bp, synthetic construct
  source 1..102: mol_type = other DNA
cgaggtcacggcgttgttttagagctacctacactcgacatcggtagctattttttttcagccaactccaatttttttttagctaccgatgtcgagtgtagg

SEQ ID NO: 450 - RNA, 14 nt, synthetic construct
  source 1..14: mol_type = other RNA
gctttcacggaagg

SEQ ID NO: 451 - RNA, 14 nt, synthetic construct
  source 1..14: mol_type = other RNA
ctgcgcatcgggtg

SEQ ID NO: 452 - RNA, 14 nt, synthetic construct
  source 1..14: mol_type = other RNA
ccgcacttccgcga

SEQ ID NO: 453 - RNA, 14 nt, synthetic construct
  source 1..14: mol_type = other RNA
gcgccaagcggtgt

SEQ ID NO: 454 - RNA, 14 nt, synthetic construct
  source 1..14: mol_type = other RNA
cgaggtcacggcgt

SEQ ID NO: 455 - RNA, 14 nt, synthetic construct
  source 1..14: mol_type = other RNA
ccgcgtggtccgtc

SEQ ID NO: 456 - RNA, 14 nt, synthetic construct
  source 1..14: mol_type = other RNA
ggggcagcgtccca

SEQ ID NO: 457 - RNA, 14 nt, synthetic construct
  source 1..14: mol_type = other RNA
ccagaccttctggc

SEQ ID NO: 458 - RNA, 14 nt, synthetic construct
  source 1..14: mol_type = other RNA
tatgcagccttaat

SEQ ID NO: 459 - RNA, 14 nt, synthetic construct
  source 1..14: mol_type = other RNA
ctgccgcctggcca

SEQ ID NO: 460 - RNA, 14 nt, synthetic construct
  source 1..14: mol_type = other RNA
aaagtgacccgaga

SEQ ID NO: 461 - RNA, 14 nt, synthetic construct
  source 1..14: mol_type = other RNA
gctgcggatgcccc

SEQ ID NO: 462 - RNA, 14 nt, synthetic construct
  source 1..14: mol_type = other RNA
gggtagaccgtccc

SEQ ID NO: 463 - RNA, 14 nt, synthetic construct
  source 1..14: mol_type = other RNA
cgagtgcggggatc

SEQ ID NO: 464 - RNA, 14 nt, synthetic construct
  source 1..14: mol_type = other RNA
gcaccaccagagat

SEQ ID NO: 465 - RNA, 14 nt, synthetic construct
  source 1..14: mol_type = other RNA
caaccttctctcct

SEQ ID NO: 466 - RNA, 14 nt, synthetic construct
  source 1..14: mol_type = other RNA
tagggtgacttgaa

SEQ ID NO: 467 - RNA, 14 nt, synthetic construct
  source 1..14: mol_type = other RNA
acgaggtcgagccg

SEQ ID NO: 468 - RNA, 14 nt, synthetic construct
  source 1..14: mol_type = other RNA
ggcccattcatcaa

SEQ ID NO: 469 - RNA, 14 nt, synthetic construct
  source 1..14: mol_type = other RNA
gctcccttgcgccc

(19) United States
(12) Patent Application Publication
(10) Pub. No.: US 2025/0388862 A1
(43) Pub. Date: Dec. 25, 2025

(54) SYSTEMS FOR CELL PROGRAMMING AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US); Nikolas George Koutis BALANIS, Chicago, IL (US); Georgina MANCINELLI, Chicago, IL (US)

(21) Appl. No.: 19/022,348

(22) Filed: Jan. 15, 2025

Related U.S. Application Data
(63) Continuation of application No. PCT/US23/28255, filed on Jul. 20, 2023.
(60) Provisional application No. 63/391,196, filed on Jul. 21, 2022.
(60) Provisional application No. 63/397,474, filed on Aug. 12, 2022.

(51) Int. Cl. C12N 5/0775; A61K 35/34; C12N 15/113

(52) U.S. Cl. CPC C12N 5/0662; A61K 35/34; C12N 15/113; C12N 2501/60; C12N 2506/45

(57) ABSTRACT

Provided herein are systems of modulating gene expression and methods of use thereof for converting cells of one type to another type (e.g. reprogramming of stem cells into tissue-specific progenitor cells).

### CROSS REFERENCE

This application is a continuation of International Patent Application No. PCT/US23/28255, filed Jul. 20, 2023, which claims the benefit of U.S. Provisional Patent Application No. 63/391,196, filed on Jul. 21, 2022, and U.S. Provisional Patent Application No. 63/397,474, filed on Aug. 12, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 15, 2025, is named 61684-703-301_SL.xml and is 375,596 bytes in size.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell. In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species.

### SUMMARY

The present disclosure provides methods and systems for programming a cell, e.g., to elicit a desired response in the cell. Systems and methods of the present disclosure can promote conversion of a cell from one type to another. Systems and methods of the present disclosure can utilize a genetic circuit to control a cascade of a plurality of desired expression and/or activity profiles of a plurality of genes in the cell to affect this conversion. Systems and methods of the present disclosure can utilize heterologous proteins and/or nucleic acid molecules as building blocks of such genetic circuit.

In an aspect of the present disclosure is a method for conversion of a plurality of pluripotent stem cells (PSCs) into a plurality of tissue-specific progenitor cells, comprising: contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to modulate expression and/or activity level of a first target endogenous gene of the plurality of distinct target endogenous genes, wherein the first target endogenous gene comprises a member selected from the group consisting of a T-box transcription factor (TBX), a basic helix-loop-helix transcription factor (bHLH), and a Paired box transcription factor (PAX); and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises a different member selected from the group consisting of the TBX, the bHLH, and the PAX, wherein, upon activation of the heterologous genetic circuit, the plurality of gate units operates to effect the conversion.

In another aspect of the present disclosure is a method for conversion of a plurality of pluripotent stem cells (PSCs) into a plurality of tissue-specific progenitor cells, comprising: contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to modulate expression and/or activity level of a first target endogenous gene of the plurality of distinct target endogenous genes, wherein the first target endogenous gene comprises a T-box transcription factor (TBX); and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises an additional TBX, wherein the TBX and the additional TBX are different types of TBX, wherein, upon activation of the heterologous genetic circuit, the plurality of gate units operates to effect the conversion.

In another aspect of the present disclosure is a method for conversion of a plurality of pluripotent stem cells (PSCs) into a plurality of tissue-specific progenitor cells, comprising: contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to reduce expression and/or activity level of a first target endogenous gene of the plurality of distinct target endogenous genes, wherein the first target endogenous gene comprises at least one cell de-differentiation factor; and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor, wherein, upon activation of the heterologous genetic circuit, the plurality of gate units operates to effect the conversion.

In another aspect of the present disclosure is a system for conversion of a plurality of pluripotent stem cells (PSCs) into a plurality of tissue-specific progenitor cells, comprising: a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to modulate expression and/or activity level of a first target endogenous gene of the plurality of distinct target endogenous genes, wherein the first target endogenous gene comprises a member selected from the group consisting of a T-box transcription factor (TBX), a basic helix-loop-helix transcription factor (bHLH), and a Paired box transcription factor (PAX); and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises a different member selected from the group consisting of the TBX, the bHLH, and the PAX, wherein, upon activation of the heterologous genetic circuit, the plurality of gate units operates to effect the conversion.

In another aspect of the invention is a system for conversion of a plurality of pluripotent stem cells (PSCs) into a plurality of tissue-specific progenitor cells, comprising: a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to modulate expression and/or activity level of a first target endogenous gene of the plurality of distinct target endogenous genes, wherein the first target endogenous gene comprises a T-box transcription factor (TBX); and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises an additional TBX, wherein the TBX and the additional TBX are different types of TBX, wherein, upon activation of the heterologous genetic circuit, the plurality of gate units operates to effect the conversion.

In another aspect of the present disclosure is a system for conversion of a plurality of pluripotent stem cells (PSCs) into a plurality of tissue-specific progenitor cells, comprising: a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises: a first gate unit that is preconfigured to reduce expression and/or activity level of a first target endogenous gene of the plurality of distinct target endogenous genes, wherein the first target endogenous gene comprises at least one cell de-differentiation factor; and a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor, wherein, upon activation of the heterologous genetic circuit, the plurality of gate units operates to effect the conversion.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of a heterologous genetic circuit. An activating moiety can initiate the heterologous genetic circuit and can activate a gate unit. A gate unit can comprise a gate moiety and/or a gene regulating moiety.

FIG. 2 provides example advantages of using a heterologous genetic circuit in cell programming.

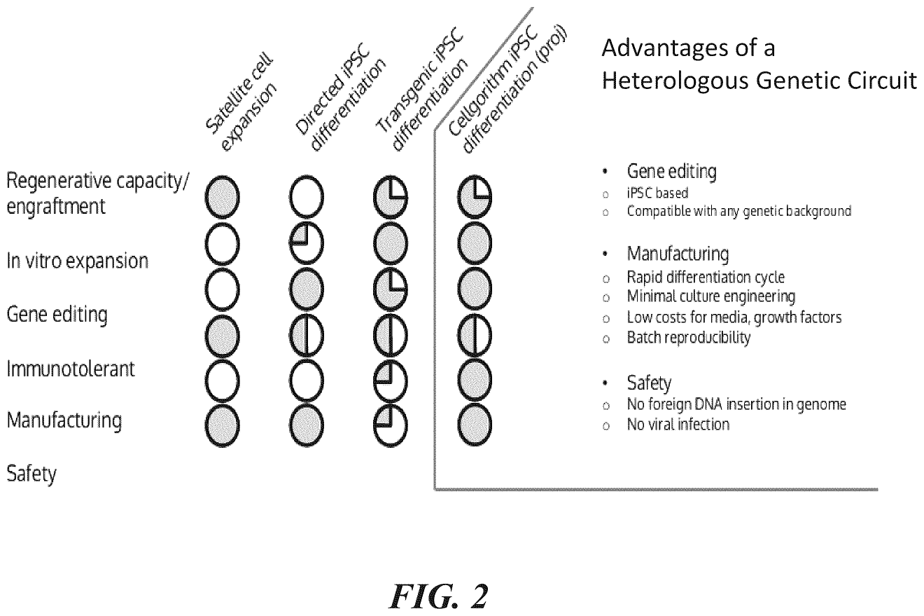

FIG. 3 schematically illustrates design of multiple heterologous genetic circuits to promote myogenic differentiation across multiple differentiation stages.

FIG. 4A schematically illustrates an example of a four-step heterologous genetic circuit.

FIG. 4B provides example heterologous genetic circuits (or conditions) to promote stepwise progressions of gene modulations in a cell to effect myoprogenitor formation.

FIG. 4C provides additional examples of heterologous genetic circuits to promote stepwise progressions of gene modulations in a cell to effect myoprogenitor formation.

FIG. 5 shows results from transient plasmid delivery of the heterologous genetic circuits.

FIG. 6 shows rate of conversion from induced pluripotent stem cells to muscle stem cells (or myoprogenitor cells) by using a heterologous genetic circuit, as compared to a control heterologous genetic circuit.

FIG. 7 shows a scatter plot (e.g., a volcano plot) to identify one or more heterologous genetic circuits that induced the stem cell-to-myogenic progenitor cell conversion.

FIG. 8 shows examples of myogenic progenitor cell marker analysis data utilized to generate the scatter plot in FIG. 7.

FIG. 9 shows myoprogenitor cell formation by myoprogenitor cells that were generated by treating stem cells with a heterologous genetic circuit as disclosed herein.

FIG. 10 schematically illustrates an example of the heterologous genetic circuit, in which activated guide nucleic acid molecules from the heterologous genetic circuit can form a complex with an endonuclease and transcriptional regulator, to modulate expression levels of target genes in a cell.

FIG. 11A schematically illustrates a portion of a polynucleotide sequence (SEQ ID NO: 61) that encodes an activatable guide nucleic acid molecule.

FIG. 11B schematically illustrates a portion of an additional polynucleotide sequence (SEQ ID NO: 449) that encodes an additional activatable guide nucleic acid molecule.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “guide nucleic acid,” “guide nucleic acid molecule,” and “gNA” as used interchangeably herein, generally refer to 1) a guide sequence that can hybridize to a target sequence or 2) a scaffold sequence that can interact with or complex with a nucleic acid guide nuclease. A guide nucleic acid can be a single-guide nucleic acid (e.g., sgRNA) or a double-guide nucleic acid (e.g., dgRNA). sgRNA can be a single RNA molecule that contains both a scaffold tracrRNA and a crRNA which can be complementary to the target sequence. Alternatively, dgRNA can be a single RNA molecule that contains a crRNA annealed to a tracrRNA through a direct repeat sequence.

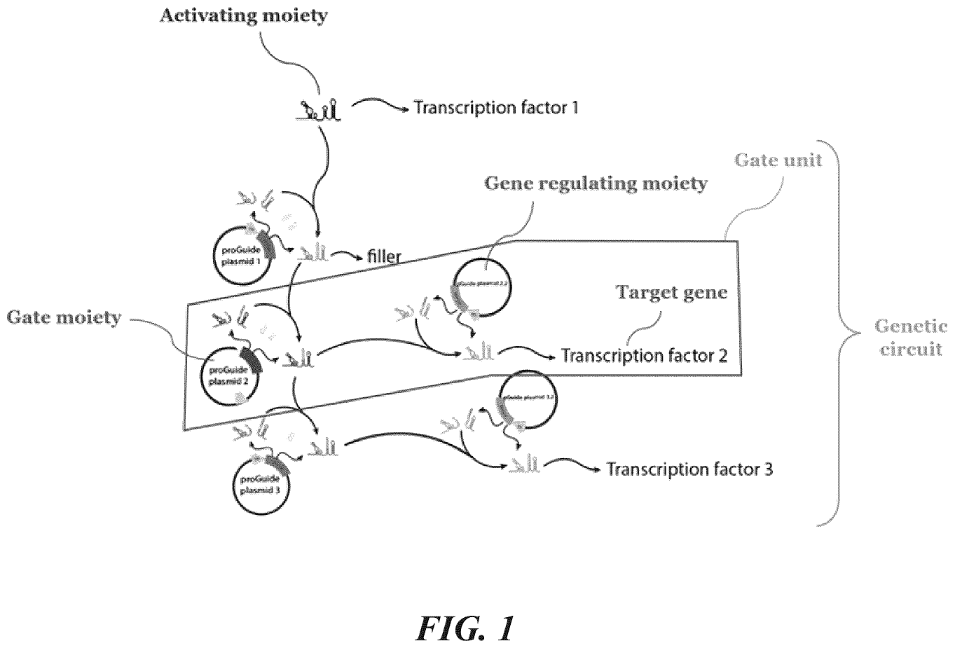

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 1).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units is activatable by an activating moiety (e.g., a heterologous input to the cell) to activate other gate units of the plurality of gate units (e.g., simultaneously at once, sequentially in a cascading manner, etc.) (FIG. 1). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 1). The terms “heterologous genetic circuit,” “HGC,” “genetic circuit,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription/translation regulatory region, or series of regions, which the genetic switch acts on. The input for a gate unit can be an activating moiety and/or another gate unit. The output for a gate unit can be used to activate another gate unit, to de-activate another gate unit, to affect a target gene, and/or a combination of any of the above. For example, a gate unit can be comprised of a plurality of gate moieties and/or a plurality of gene regulating moieties (FIG. 1).

The term “activating moiety,” as referred to herein, generally refers to a moiety that can activate plurality of genetic circuits and/or a plurality of gate units. An activating moiety can be a heterologous input to a cell. In some cases, activating moieties can include, but are not limited to, a guide nucleic acid molecule (e.g., a gRNA) or other nucleic acid, polypeptides, polynucleotides, small molecules, light, or a combination thereof. For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties. The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively or in addition to, a gate moiety can activate and/or deactivate another gate unit of the genetic circuit (FIG. 1). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 1). For example, a gene editing moiety can regulate expression of a gene by editing a nucleic acid sequence (e.g. CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g. CRISPR-Cas-inspired RNA targeting systems). Alternatively, a gene editing moiety can repress translation of a gene (e.g. Cas13).

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g. Cas12).

In some cases, a plasmid can encode a non-functional form of a gene editing moiety. The plasmid can be activated (e.g., genetically modified) to express a functional form of the gene editing moiety, e.g., via activation of a functional gate moiety. For example, the plasmid can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited (e.g., cleaved at one or more sites, then repaired via endogenous mechanisms (e.g., homologous recombination, nonhomologous end joining) to allow expression of a functional form of the gene editing moiety (e.g., a functional form of the guide nucleic acid molecule with specific binding to the target gene of the cell), to permit modulation of the target gene in the cell.

In some cases, a gene regulating moiety can comprise a nucleic acid molecule (e.g., a guide nucleic acid molecule that forms a complex with an endonuclease, such as a Cas protein). Alternatively or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

The abovementioned Cas proteins can form a complex with a guide nucleic acid (gNA (e.g., a guide RNA (gRNA)) and utilize the gNA to specifically bind to a target polynucleotide sequence (e.g., a target DNA sequence, a target RNA sequence). Accordingly, in some cases, such Cas proteins may be referred to as a “NA-guided nuclease” (e.g., RNA-guided nuclease). As used herein, the term “guide nucleic acid” (gNA) can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence”. A nucleic acid-targeting segment may comprise a sub-segment that may be referred to as a “protein binding segment” or “protein binding sequence” or “Cas protein binding segment” or “scaffold sequence.”

A gene regulating moiety can be a transcriptional modulator system (e.g., a gene repressor complex or a gene activator complex). For example, a gene regulating moiety can be a gene repressor complex comprising a dCas protein operatively coupled to (e.g., coupled to or fused with) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASC1, JMJD2D, JARID1A, RBP2, JARID1B/PLU-1, JARIDIC/SMCX, JARIDID/SMCY, HDACl, HDAC2, HDAC3, HDAC8, HDAC4, HDAC5, HDAC7, HDAC9, SIRT1, SIRT2, HDAC11, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SET1A, SET1B, MLL1, MLL2, MLL3, MLL4, MLL5, ASH1, SYMD2, NSD1, JHDM2a, JHDM2b, UTX, JMJD3, GCN5, PCAF, CBP, p300, TAF1, TIP60/PLIP, MOZ/MYST3, MORF/MYST4, SRCl, ACTR, P160, CLOCK, TET1CD, TET1, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TET1CD), TET1, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. During transient expression, episomal DNA can be transferred to daughter cells, but since episomal DNA is not replicated, it is not permanently heritable and will dilute out over time. In contrast, stable expression of a transfected gene can occur when the gene is co-transfected with another gene that confers a selection advantage to the transfected cell. During stable expression, plasmids can have a DNA replication element that allows them to be inherited or integrated into the genome. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink Deoxy Nucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and may not, under normal circumstances, differentiate into a different cell type or revert to a less differentiated cell type.

The term “dedifferentiation” or “de-differentiation” generally refers to a process by which a specialized, committed, or partially specialized cell loses the features of the specialized cell (e.g., a muscle cell). A dedifferentiated cell or dedifferentiation-induced cell is one that has taken on a less specialized position within the lineage of a cell (e.g., a stem cell or a progenitor cell). A dedifferentiated cell (e.g., a stem cell or a progenitor cell) can subsequently differentiate into a different cell type or can revert to a less differentiated cell type.

The term “pluripotent” generally refers to the ability of a cell to form all lineages of the body or soma (e.g., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (e.g., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., muscle cells). In some cases, iPSCs can be engineered to differentiate first into tissue-specific stem cells (e.g., mesenchymal stem cells), which can be further induced to differentiate into committed cells (e.g., muscle cells).

The term “embryonic stem cell” (ESCs) generally refers to cells derived from the naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiate directly into committed cells (e.g., muscle cells). In some cases, ESCs can be engineered to differentiate first into tissue-specific stem cells (e.g., mesenchymal stem cells), which can be further induced to differentiate into committed cells (e.g., muscle cells).

The term “isolated stem cells” generally refers to any type of stem cells disclosed herein (e.g., ESCs, HSCs, mesenchymal stem cells (MSCs), etc.) that are isolated from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The terms “muscle cell” or “myocyte” generally refer to a cell that comprises muscle tissue. Non-limiting examples of muscle cells include cardiac muscle cells, smooth muscle cells, and skeletal muscle cells. Skeletal muscle cells are multinucleated and can also be referred to as muscle fibers. Muscle cells develop from myogenic stem cells, including but not limited to muscle stem cells and myoblasts. The terms “muscle stem cell” or “MuSC” as used interchangeably herein generally refer to muscle-specific progenitor cells that are more committed than stem cells (e.g., ESCs, MSCs, iPSCs, etc.) but less differentiated than myoblasts. MuSCs can be myogenic progenitor cells or myoprogenitor cells, as used interchangeably herein. MuSCs can be generated ex vivo by engineering isolated stem cells. MuSCs can be generated in vivo by engineered stem cells in vivo, e.g., by administering such stem cells with any one of the heterologous genetic circuits disclosed herein. MuSCs engineered from stem cells as disclosed herein can be similar to (e.g., transcriptionally as assessed by RNA sequencing, morphologically, etc.) an isolates muscle satellite cell, such as a quiescent satellite cell.

### Overview

Biological programming, such as cellular programming, allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control the cell fate of a cell or plurality of cells by inducing differentiation or dedifferentiation and converting from one cell type to another. Cellular programming is controlled through the regulation of desired expression and/or activity levels of a plurality of genes in a cell.

Although CRISPR/Cas systems are widely used for gene editing, Cas is essentially a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this simple barcoding, often using exogenous fluorophores, doesn't allow for the regulation of endogenous genes to effect cell differentiation.

Further, differentiation or dedifferentiation of cells is currently enabled through the use of exogenous serums and growth factors which bypass the underlying machineries of a cell's programming. The use of exogenous serums, growth factors, and other similar methods results in cells that are instructed to differentiate, but which lack the concomitant underlying biology (e.g., chromatin in the correct state, etc.) This lack often results in cells that either undergo premature termination of differentiation into non-desired cell types or which undergo inefficient differentiation whereby there are low yields of target cell types, or the resulting desired differentiated cells are only semi-functional. Semi-functional cells may resemble cell types of interest, but may lack key biological features necessary for the normal function of the desired differentiated cell type.

Thus, there remains an unmet need for an activatable, CRISPR/Cas system and use of the same to edit a target polynucleotide (e.g., a genome of a cell, in particular a eukaryotic cell), using cascades of gRNAs to form genetic circuits in order to single-handedly affect gene regulation and, in turn, cell-fate determination without the use of serums and exogenous growth factors. The preprogrammed, activatable, and self-regulating gRNA cascade CRISPR/Cas system finds use, e.g., in gene therapy, genetic circuitry, and/or complex cell-fate determination and/or control.

The present disclosure provides systems and methods for engineering a CRISPR/Cas9 system, which includes a Cas endonuclease and an array of cognate single guide RNAs (sgRNA or gRNA) that harbor inactivation sequences in a non-essential region and are activatable, to allow for modulation and modification of that system without the need for serum, growth factors, or other additional exogenous signals. The present disclosure also provides for an engineered cell that can contain any of the above-mentioned systems or that can be capable of performing any of the above-mentioned methods.

### Systems and Methods for Conversion of Cells of One Type to Cells of Another Type

Various aspects of the present disclosure provide systems for inducing a desired conversion from one type of cells into another type of cells. To this end, various aspects of the present disclosure provide methods for inducing a desired expression and/or activity levels (or profiles thereof) of one or more target genes in a cell.

In an aspect, the present disclosure provides for a system that converts a plurality of cells of a first type into a plurality of cells of a second cell type. The system can comprise a heterologous genetic circuit comprising a plurality of gate units. The plurality of gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s). The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). The plurality of gate units can be different (e.g., comprising different polynucleotide sequences). Each gate unit of the plurality of gate units can affect the modulation of the expression and/or activity levels of a distinct target gene or a plurality of distinct target genes.

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel sequential paths), or a combination thereof.

A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g. cell proliferation assays or metabolic activity assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

A plurality of gate units as disclosed herein can be sufficient to effect the conversion of a plurality of cells of a first cell type into a plurality of cells of a second cell type. For example, a plurality of gate units as disclosed herein can be sufficient to effect the conversion from a plurality of pluripotent stem cells (PSCs) into a plurality of tissue-specific progenitor cells. Alternatively, a plurality of gate units as disclosed herein can be necessary but insufficient to effect the conversion of a plurality of cells of a first cell type into a plurality of cells of a second cell type.

The outcome in the cell can comprise regulation of a distinct target gene or set of distinct target genes. The plurality of gate units can induce distinct modulations of the plurality of target genes (e.g., in a sequential manner), such that a collection of the modulations of the genes in concert yield a final expression and/or activity profile of the cell. The final expression and/or activity level profile of the cell can exemplify an outcome, such as a conversion of the cell from one cell type to another (or a process thereof).

In some cases, of the plurality of gate units, as disclosed herein, can be necessary but individually insufficient to effect the desired expression and/or activity profile of the target cell. Thus, the outcome in the cell (e.g., enhanced cell function, induced cell state, etc.) induced by the plurality of gate units may not be possible in absence of any one of the plurality of gate units. Alternatively, a degree or measure of the outcome in the cell induced by the plurality of gate units can be different (e.g., greater for a positive marker, or less for a negative marker) than a degree or measure of the outcome in a control cell that is induced by none, one or more, but not all of the plurality of gate units, and/or by all of the plurality gate units occurring through a different sequential order of events.

A second gate unit can be activated by a first gate unit (e.g. directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.). Yet in another alternative, the first gate unit and the second gate unit can be activated by different activating moieties (e.g., different polynucleotide molecules, such as different guide nucleic acid molecules).

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively, or in addition to, inactivation can be achieved through the use of a transcriptional modulator system (e.g. a transcriptional repressor). Alternatively, or in addition to, inactivation can be achieved through CRISPRi steric hindrance without the necessity of an additional transcriptional modulator. An endonuclease-transcriptional modulator system (e.g., a Cas-repressor) can be used to achieve polynucleotide cleavage (e.g. for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively or in addition to, the endonuclease-transcriptional modulator system (e.g., a Cas-repressor) can be used to modulate target gene expression. Alternatively, or in addition to, a CAS transcriptional modulator system that lacks endonuclease activity (dCAS or bare CAS with a shortened spacer insufficient to support cleavage) targets to a DNA region an physically halts transcription elongation resulting in repression of the target gene (CRISPRi).

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

In some cases, a first gate unit modulates a first target gene. Alternatively, or in addition to, a first gate unit can also modulate a second gate unit. The modulation of the second gate unit can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or after the modulation of the first gate unit, as ascertained by rt-qPCR, Western blotting, or other methods.

In some cases, the second gate unit can modulate a second target gene. The modulation of the second target gene can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or more after the modulation of the first target gene, as ascertained by rt-qPCR, Western blotting, or other methods.

In some cases, modification of a target gene by a gate unit can inactivate a gene. For example, modification of a gene can stop expression and/or activity level of a target gene. Alternatively, modification of a gene can decrease the expression and/or activity level of a target gene. In some cases, modification of a gene can increase the expression and/or activity level of a target gene. Alternatively, modification of a gene can maintain the expression and/or activity level of a target gene.

An expression and/or activity profile of a gene of interest (e.g. a differentiation marker) can be compared to a control gene (e.g., a house keeping gene such as GAPDH), relative expression levels of two or more genes of interest (e.g., a ratio of expression or activity level between a stem cell marker and a differentiation marker), relative average expression levels of a gene of interest compared to average expression levels of that same gene of interest in a cell type of interest, etc.

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least or up to about 1 gate moiety, at least or up to about 2 gate moieties, at least or up to about 3 gate moieties, at least or up to about 4 gate moieties, at least or up to about 5 gate moieties, etc.) and/or a gene regulating moiety (e.g., at least or up to about 1 gene regulating moiety, at least or up to about 2 gene regulating moieties, at least or up to about 3 gene regulating moieties, at least or up to about 4 gene regulating moieties, at least or up to about 5 gene regulating moieties, at least or up to about 6 gene regulating moieties, at least or up to about 7 gene regulating moieties, at least or up to about 8 gene regulating moieties, at least or up to about 9 gene regulating moieties, at least or up to about 10 gene regulating moieties, etc.). A gate moiety as disclosed herein can comprise a guide nucleic acid molecule (gNA) (e.g., at least or up to about 1 gNA molecule, at least or up to about 2 gNA molecules, at least or up to about 3 gNA molecules, at least or up to about 4 gNA molecules, at least or up to about 5 gNA molecules, etc.). A gene regulating moiety as disclosed herein can comprise a gNA (e.g., at least or up to about 1 gNA molecule, at least or up to about 2 gNA molecules, at least or up to about 3 gNA molecules, at least or up to about 4 gNA molecules, at least or up to about 5 gNA molecules, etc.). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety may activate the initial gate unit without directly binding the at least the portion of the initial gate unit (e.g., through the use of electromagnetic energy). In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, a vector (or expression cassette) encoding the activatable gNA can comprise an inactivation polynucleotide sequence to render the gNA inactive until activated (e.g., until the inactivation polynucleotide sequence is modified or removed from the vector. For example, the inactivation polynucleotide sequence can encode a self-cleaving polynucleotide molecule (e.g., a ribozyme). Alternatively or in addition to, the inactivation polynucleotide sequence can encode non-canonical transcription termination sequence, as described below. The inactivation polynucleotide sequence can be a part of or adjacent to a region of the vector that encodes (i) a spacer sequence of the gNA, (ii) a scaffold sequence of the gNA, and/or (ii) any linker sequence between the spacer sequence and the scaffold sequence. The vector can comprise at least or up to about 1 inactivation polynucleotide sequence, at least or up to about 2 inactivation polynucleotide sequences, at least or up to about 3 inactivation polynucleotide sequences, at least or up to about 4 inactivation polynucleotide sequences, at least or up to about 5 inactivation polynucleotide sequences, at least or up to about 6 inactivation polynucleotide sequences, at least or up to about 7 inactivation polynucleotide sequences, at least or up to about 8 inactivation polynucleotide sequences, at least or up to about 9 inactivation polynucleotide sequences, or at least or up to about 10 inactivation polynucleotide sequences.

In some cases, the term “proGuide” as generally used herein may refer to such vector (e.g., a plasmid) that encodes the activatable gNA. The proGuide can be an example of a gate moiety. The proGuide can be an example of a gene regulating moiety.

In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the activatable gNA molecule comprises a non-canonical transcription termination sequence (e.g., a polyX sequence, such as a polyU sequence or a polyT sequence), such that a functional gNA molecule is not expressed until a gene encoding the activatable gNA having the non-canonical transcription termination sequence can be modified (e.g., to remove some or all of the transcription termination sequence). Thus, in absence of the modification of the transcription termination sequence, a non-functional variant (e.g., a non-functional fragment) of the gNA may be expressed. In some embodiments, the gNA can be synthetic. In some embodiments, the gNA can have a fluorescent label attached.

In some cases, a size of the polyT sequence is greater than or equal to a threshold length, wherein the threshold length is sufficient to reduce expression of the guide nucleic acid molecule from the polynucleotide sequence. Accordingly, a plasmid (e.g., a gate moiety or a gene regulating moiety) can encode an inactivated gNA comprising the polyT sequence that is greater than or equal to the threshold length, and editing of such plasmid to reduce the length of the polyT to below the threshold length can permit expression of the gNA in its entirety without early termination, thereby activating the gNA. In some cases, the polyT sequence comprises at least 5 T. In some cases, the polyT sequence comprises at least 7 T. In some cases, the polyT sequence comprises at least 8 T. In some cases, the polyT sequence comprises at least 10 T. In some cases, the polyT sequence comprises between 5 T and 15 T. In some cases, the polyT sequence comprises one or more additional nucleotides that are not T.

In some cases, a gene regulating moiety (e.g., a guide nucleic acid and/or an endonuclease) can be configured to bind to a target polynucleotide sequence operatively coupled to a target gene in a cell. The target gene can comprise an encoding polynucleotide sequence that encodes a target nucleic acid molecule or a target protein. The target polynucleotide sequence can be a part of the encoding polynucleotide sequence. Alternatively, the target polynucleotide sequence may not be a part of the encoding polynucleotide sequence. For example, the target polynucleotide sequence can be upstream of the encoding polynucleotide sequence (e.g., part of a promoter of the encoding polynucleotide sequence, such as a transcription start site (TSS).

As provided herein, when the heterologous genetic circuit is activated to induce a plurality of distinct modulations of a target gene, as provided herein, the plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. Alternatively or in addition to, the distinct modulation of the target gene can be substantially the same (e.g., the same).

The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least about 1, at least about 2, at least about 3, at least about 4, at least about 5, at least about 6, at least about 7, at least about 8, at least about 9, at least about 10, or more single-stranded breaks. In some cases, inactivation of a gene can be caused by at most about 10, at most about 9, at most about 8, at most about 7, at most about 6, at most about 5, at most about 4, at most about 3, at most about 2, or at most about 1 single-stranded breaks.

In some cases, a gNA can have a size (e.g., including both spacer sequence and scaffold sequence) of at least or up to about 60 nucleotides, at least or up to about 70 nucleotides, at least or up to about 80 nucleotides, at least or up to about 85 nucleotides, at least or up to about 90 nucleotides, at least or up to about 95 nucleotides, at least or up to about 100 nucleotides, at least or up to about 105 nucleotides, at least or up to about 110 nucleotides, at least or up to about 120 nucleotides, at least or up to about 130 nucleotides, at least or up to about 140 nucleotides, at least or up to about 150 nucleotides, or at least or up to about 200 nucleotides.

In some cases, a scaffold sequence of a gNA can have a size of at least or up to about 30 nucleotides, at least or up to about 35 nucleotides, at least or up to about 40 nucleotides, at least or up to about 45 nucleotides, at least or up to about 50 nucleotides, at least or up to about 55 nucleotides, at least or up to about 60 nucleotides, at least or up to about 65 nucleotides, at least or up to about 70 nucleotides, at least or up to about 75 nucleotides, at least or up to about 80 nucleotides, at least or up to about 85 nucleotides, at least or up to about 90 nucleotides, at least or up to about 95 nucleotides, at least or up to about 100 nucleotides, at least or up to about 100 nucleotides, at least or up to about 120 nucleotides, at least or up to about 130 nucleotides, at least or up to about 140 nucleotides, or at least or up to about 150 nucleotides.

In some cases, a spacer sequence of a gNA can have a size of at least or up to about 10 nucleotides, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30 nucleotides.

In some cases, the systems and methods of the present disclosure can utilize a single endonuclease system (e.g., a Cas-repressor) to achieve both (i) polynucleotide cleavage (e.g. for activating/inactivating the gate moiety and/or the gene regulating moiety) and (ii) modulation of target gene expression. When using a single endonuclease-transcriptional modulator system, unique guide nucleic acid molecules (gNAs) of differing spacer sequence lengths can be used to determine whether the single endonuclease-transcriptional modulator system may (i) hybridize to the polynucleotide sequence to induce Cas-mediated nuclease activity of the polynucleotide sequence, or (ii) can hybridize to a target gene (e.g., genomic DNA) to modulate expression and/or activity level of the target gene via action of the transcriptional activator without mediating Cas nuclease activity, as desired by the individual heterologous genetic circuit. For example, use of gNAs of differing spacer sequence lengths that bind to different targets can allow for a second gate unit as provided herein to induce inactivation of a first gate unit that has been activated and/or induce a distinct modulation of a second target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g. a Cas or a Cas-transcriptional modulator fusion protein) at a target polynucleotide sequence. Alternatively or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g. a Cas-activator system, a Cas-repressor system), without cleavage of the target gene.

In some cases, modification of a polynucleotide sequence (e.g., as a component of a gate unit, such as a gate moiety) or a target gene can be caused by a double-stranded break wherein there is a discontinuity in both nucleotide strands. In some cases, a number of such double-stranded break (e.g., necessary for such modification) can be at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10.

In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein. Alternatively or in addition not, the gene regulating moiety can induce epigenetic modification (or epigenome modification) as provided herein.

In some cases, the modification of the polynucleotide sequence or the target gene, as provided herein, can inactivate the polynucleotide sequence or the target gene. For example, modification of the polynucleotide sequence or the target gene can repress or reduce expression and/or activity level of the polynucleotide sequence or the target gene. In some cases, the modification of the polynucleotide sequence or the target gene, as provided herein, can activate the polynucleotide sequence or the target gene. For example, modification of the polynucleotide sequence or the target gene can increase expression and/or activity level of the polynucleotide sequence or the target gene.

In some cases, the modification of the polynucleotide sequence or the target gene, as provided herein, can comprise decreasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, at least or up to about 99%, or about 100% (e.g., as compared to a control that, for example, lacks the modification).

In some cases, the modification of the polynucleotide sequence or the target gene, as provided herein, can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, or at least or up to about 100-fold (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 150%, at least or up to about 200%, at least or up to about 300%, at least or up to about 400%, or at least or up to about 500% (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, at least or up to about 100-fold, at least or up to about 200-fold, at least or up to about 300-fold, at least or up to about 400-fold, at least or up to about 500-fold, or at least or up to about 1,000-fold (e.g., as compared to a control that, for example, lacks the modification).

In some embodiments of any one of the systems disclosed herein, the gNA of the gate moiety and/or the gene regulating moiety (e.g., a gNA encoded by the gate moiety and/or the gene regulating moiety) can comprise a spacer sequence. In some cases, the spacer sequence can exhibit specific binding to a target gene (e.g., an endogenous target gene). Alternatively, the spacer sequence can be agnostic to the target gene, but rather can exhibit specific binding to a target polynucleotide sequence of another gate moiety or another gene regulating moiety. Example spacer sequences can be found in Table 1.

Non-limiting examples of the one or more target genes can comprise TBXT, bHLH (e.g., MSGN1), and/or PAX (e.g., PAX3, PAX7). Non-limiting examples of the one or more target genes can comprise TBXT, MSGN1, PAX3, and/or PAX7. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-31 (e.g., such sequence identity to one or more members selected from SEQ ID NOs: 1-6 for TBXT targeting, SEQ ID NOs: 7-12 for TBXT6 targeting, SEQ ID NOs: 13-18 for MSGN1 targeting, SEQ ID NOs: 19-25 for PAX3 targeting, and SEQ ID NOs: 26-31 for PAX7 targeting), or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-31 (e.g., such sequence identity to one or more members selected from SEQ ID NOs: 1-6 for TBXT targeting, SEQ ID NOs: 7-12 for TBXT6 targeting, SEQ ID NOs: 13-18 for MSGN1 targeting, SEQ ID NOs: 19-25 for PAX3 targeting, and SEQ ID NOs: 26-31 for PAX7 targeting), or a complementary sequence thereof (e.g., with uracil-to-thymine conversion).

TABLE 1
Example Spacer Sequences of guide nucleic acid
molecules

| SEQ ID NO | Description | Guide RNA sequence |
|---|---|---|
| 450 | TBXT | GCUUUCACGGAAGG |
| 451 | TBXT | CUGCGCAUCGGGUG |
| 452 | TBXT | CCGCACUUCCGCGA |
| 453 | TBXT | GCGCCAAGCGGUGU |
| 5 | TBXT | AUCUCGCAGCACCG |
| 6 | TBXT | CGCACUUCCGCGAG |
| 7 | TBXT6 | AUCUCGCAGCACCG |
| 8 | TBXT6 | GGGAGCCCCGGCUA |
| 454 | TBXT6 | CGAGGUCACGGCGU |
| 455 | TBXT6 | CCGCGUGGUCCGUC |
| 456 | TBXT6 | GGGGCAGCGUCCCA |
| 457 | TBXT6 | CCAGACCUUCUGGC |
| 13 | MSGN1 | CAAAGUGACCCGAG |
| 14 | MSGN1 | GGGCAUCCGCAGCA |
| 458 | MSGN1 | UAUGCAGCCUUAAU |
| 459 | MSGN1 | CUGCCGCCUGGCCA |
| 460 | MSGN1 | AAAGUGACCCGAGA |
| 461 | MSGN1 | GCUGCGGAUGCCCC |
| 19 | PAX3 | GUGACACCGAGUGC |
| 20 | PAX3 | AGGGCGGAAAAGUU |
| 21 | PAX3 | AGGCCCCGGAGUCC |
| 462 | PAX3 | GGGUAGACCGUCCC |
| 463 | PAX3 | CGAGUGCGGGGAUC |
| 464 | PAX3 | GCACCACCAGAGAU |
| 465 | PAX3 | CAACCUUCUCUCCU |
| 26 | PAX7 | GGGGCCUGGUCUCC |
| 27 | PAX7 | ACUGCAGCCAGGGG |
| 466 | PAX7 | UAGGGUGACUUGAA |
| 467 | PAX7 | ACGAGGUCGAGCCG |
| 468 | PAX7 | GGCCCAUUCAUCAA |
| 469 | PAX7 | GCUCCCUUGCGCCC |

The gene regulating moiety as disclosed herein can comprise an endonuclease, such as a CRISPR-Cas protein exhibiting at least a portion of its nuclease activity. For example, the nuclease activity can be utilized to activate expression or activity of a guide nucleic acid molecule, thereby to activate at least a portion of the heterologous genetic circuit as described herein.

The gene regulating moiety as disclosed herein can comprise an endonuclease that is operatively coupled to a transcriptional effector, including a transcriptional activator or a transcriptional repressor, that is heterologous to the cell. The endonuclease may be naturally or may be engineered to exhibit reduced nuclease activity (or substantially no nuclease activity), such that the endonuclease can be used to specifically bind to a target gene, but without cleaving the target gene (e.g., endogenous gene, such as TBX, bHLH, PAX, etc.). In some cases, the nuclease can be a deactivated Cas (dCas). Instead, once the endonuclease identifies and binds the target gene, the transcriptional effector that is coupled (e.g., covalently or non-covalently coupled) to the endonuclease can interact with the target gene to either increase or decrease the expression level of the target gene, thereby either increasing or decreasing the activity level of the target gene. For example, the endonuclease and the transcriptional effector can be part of a fusion protein that is encoded by the same expression cassette.

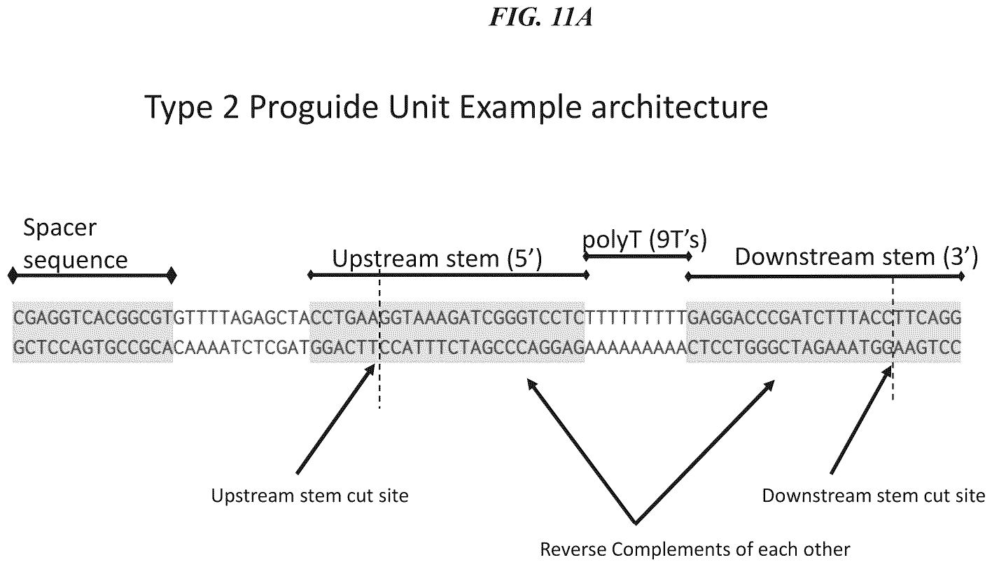

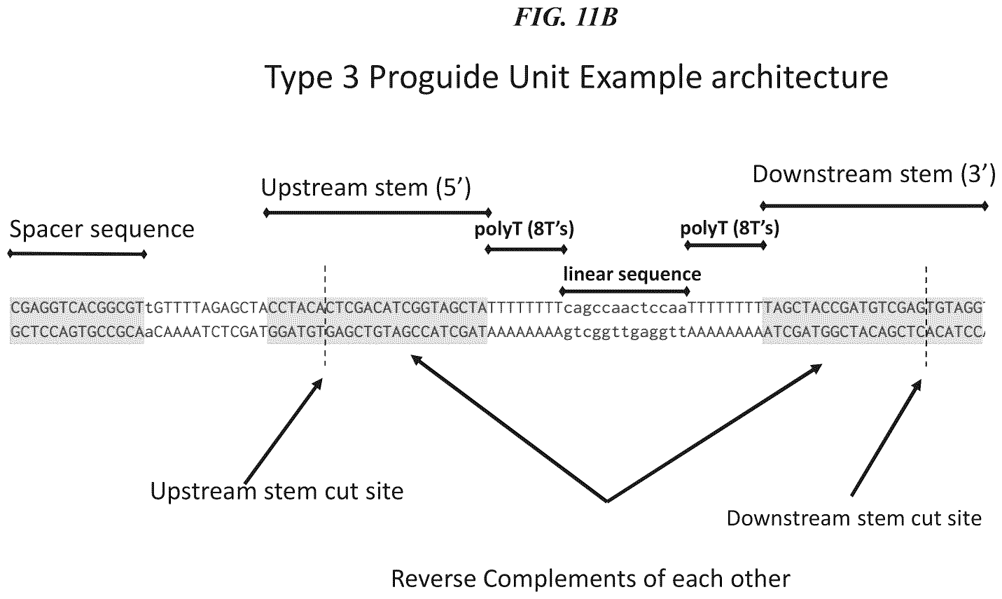

A proGuide as provided herein can encode an activatable guide nucleic acid molecule, e.g., having the inactivation polynucleotide sequence (e.g., one or more polyX sequences, such as one or more polyT sequences). In some embodiments, a portion of the proGuide encoding the activatable guide nucleic acid molecule can comprise various regions that are sequentially linked, comprising a spacer sequence, an extra sequence (e,g, a linker sequence), an upstream stem, a poly T unit, and a downstream stem, as shown in TABLE 2 and TABLE 4. In some cases, upon modification or removal of the polyT unit, the upstream stem and the downstream stem can form a part of a scaffold sequence of a functional guide nucleic acid molecule. These various regions can be sequentially linked, e.g., from 5′ to 3′, in the order as illustrated in FIGS. 11A and 11B. In some examples, a sequentially linked proGuide as reflected in FIG. 11A can have the following sequence: CGAGGTCACGGCGTGTTTTAGAGCTACCTGAAGGTAAAGATCGGGTCCTCTTTTTTTTTGA GGACCCGATCTTTACCTTCAGG (SEQ ID NO: 61). In some examples, a sequentially linked proGuide as reflected in FIG. 11B can have the following sequence: CGAGGTCACGGCGTtGTTTTAGAGCTACCTACACTCGACATCGGTAGCTATTTTTTTTcagcca actccaaTTTTTTTTTAGCTACCGATGTCGAGTGTAGG (SEQ ID NO: 449). A set of proGuides in a common heterologous genetic circuit can have identical (or substantially the same) or different extra sequences disposed between the spacer sequence and the upstream stem. In some examples, the proGuides as partly provided in TABLE 2 and TABLE 4 can have the following identical extra sequence: GTTTTAGAGCTA (SEQ ID NO: 32). Alternatively, the proGuide(s) may comprise a different extra sequence, e.g., as a linker. Yet in another alternative, the proGuide(s) may not comprise any extra/linker sequence between the spacer sequence and the upstream stem region. In some examples, the proGuides as partly provided in TABLE 2 can have the following identical polyT sequence: TTTTTTTTT. Alternatively, or in addition to, the proGuides as partly provided in TABLE 4 can have the following identical polyT sequence: TTTTTTTTcagccaactccaaTTTTTTTT (SEQ ID NO: 34).

In some embodiments, a proGuide can comprise a linker sequence between (i) a domain encoding a spacer sequence of a guide nucleic acid and (ii) a domain comprising a scaffold sequence of the guide nucleic acid, which domain comprising one or more inactivation polynucleotide sequences (e.g., one or more polyT sequences). The linker sequence as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to SEQ ID NO: 32, or a complementary sequence thereof. Alternatively, the proGuide may not comprise a linker sequence between the two domains (i) and (ii).

In some embodiments, a proGuide can comprise inactivation polynucleotide sequence that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to TTTTTTTTT or SEQ ID NO:44, or a complementary sequence thereof.

In some embodiments, a proGuide can comprise a target polynucleotide domain at or adjacent to an inactivation polynucleotide sequence (e.g., at or adjacent to 5′ and/or 3′ ends of the inactivation polynucleotide sequences), which target polynucleotide domain can be targeted (e.g., via sequential activation mechanism of the heterologous genetic circuit as provided herein) to modify (e.g., edit, cleave) the inactivation polynucleotide sequence, thereby rendering the proGuide to express an activated guide nucleic acid molecule. The target polynucleotide domain of a proGuide may not exhibit sequence identity to any comparable endogenous polynucleotide sequence in a cell, thereby to avoid inadvertent targeting and modulation of an endogenous target gene. Non-limiting examples of such target polynucleotide domain of a proGuide can include one or more polynucleotide sequences from SEQ ID NOs: 35-40 and 431-448. In some cases, a target polynucleotide domain of a proGuide can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members from SEQ ID NOs: 35-40 and 431-448, or a complementary sequence thereof.

In some embodiments, the inactivation polynucleotide sequence of the proGuide can be disposed between two target polynucleotide domains, which may or may not be targetable by a common guide nucleic acid sequence. In some cases, the two target polynucleotide domains can be reverse and complementary to one another, such that the inactivation polynucleotide sequence can be modified or cleaved by the same mechanism (e.g., same spacer sequence of a guide nucleic acid molecule). For example, the two target polynucleotide domains of the proGuide can be one or more pairs from the following (or a comparable paid exhibiting partial or full sequence identity to the following): SEQ ID NO: 35 and SEQ ID NO: 38, SEQ ID NO: 36 and SEQ ID NO: 39, SEQ ID NO: 37 and SEQ ID NO: 40, SEQ ID NO: 431 and SEQ ID NO: 440, SEQ ID NO: 432 and SEQ ID NO: 441, SEQ ID NO: 433 and SEQ ID NO: 442, SEQ ID NO: 434 and SEQ ID NO: 443, SEQ ID NO: 435 and SEQ ID NO: 444, SEQ ID NO: 436 and SEQ ID NO: 445, SEQ ID NO: 437 and SEQ ID NO: 446, SEQ ID NO: 438 and SEQ ID NO: 447, and SEQ ID NO: 439 and SEQ ID NO: 448, or a complementary sequence thereof.

In some embodiments, a proGuide can comprise a polynucleotide sequence that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members from SEQ ID NOs: 101-112 (e.g., TBXT targeting), SEQ ID NOs: 113-124 (e.g., TBX6 targeting), SEQ ID NOs: 125-136 (e.g., MSGN1 targeting), SEQ ID NOs: 137-148 (e.g., PAX3 targeting), SEQ ID NOs: 149-160 (e.g., PAX7 targeting), SEQ ID NOs: 161-196 (e.g., TBXT targeting), SEQ ID NOs: 197-232 (e.g., TBX6 targeting), SEQ ID NOs: 233-268 (e.g., MSGN1 targeting), SEQ ID NOs: 269-304 (e.g., PAX3 targeting), and SEQ ID NOs: 305-309 and 400-430 (e.g., PAX7 targeting), or a complementary sequence thereof.

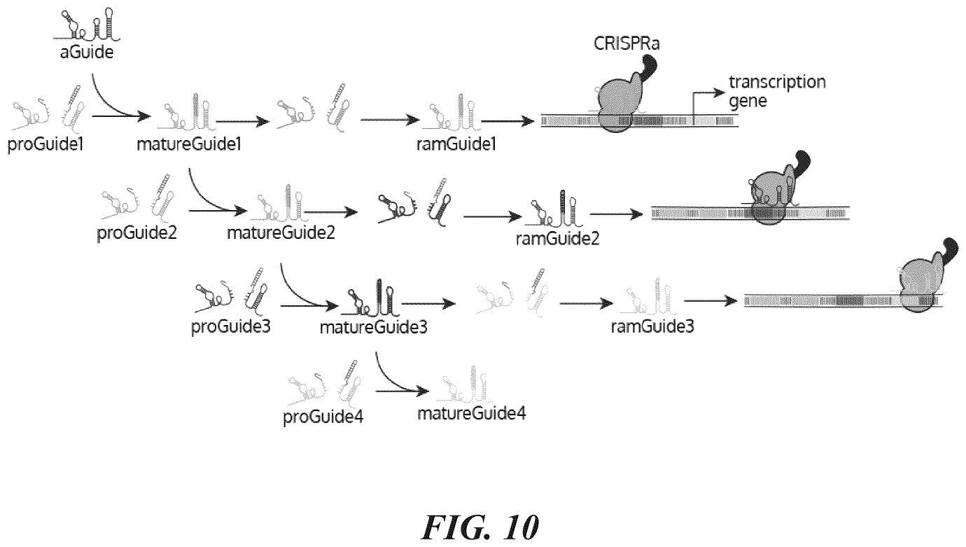

FIG. 10 schematically illustrates use of the heterologous genetic circuit in conjunction with an endonuclease-transcriptional effector fusion (e.g., CRISPR Cas-transcriptional activator, such as Cas9-VPR). Each gate moiety (denoted as proGuide in FIG. 10) can be a modified self-deactivating (e.g., self-destructing) guide RNA, which would be configured to form a complex with the CRISPR Cas-transcriptional effector fusion if not deactivated. The initial activating moiety (denoted as activating guide RNA or “aGuide”) can convert the first gate moiety to an activated guide RNA (denoted as a matureGuide). Each matureGuide can target an additional gene regulating moiety encoding an activatable guide RNA against a target gene (denoted as ramGuide), to activate such ramGuide. Subsequently, the activated ramGuide can form a complex with the CRISPR Cas-transcriptional effector fusion protein to bind the target gene and regulate expression level of the target gene. The activated matureGuide can also target an additional gate moiety that is downstream within the heterologous genetic circuit's signaling cascade, to subsequently regulate expression of one or more additional genes.

In some embodiments, the transcriptional effector can be a histone epigenetic modifier (or a histone modifier). In some cases, the histone epigenetic modifier can modulate histones through methylation (e.g., a histone methylation modifier, such as an amino acid methyltransferase, e.g., KRAB). In some cases, the histone epigenetic modifier can modulate histones through acetylation. In some cases, the histone epigenetic modifier can modulate histones through phosphorylation. In some cases, the histone epigenetic modifier can modulate histones through ADP-ribosylation. In some cases, the histone epigenetic modifier can modulate histones through glycosylation. In some cases, the histone epigenetic modifier can modulate histones through SUMOylation. In some cases, the histone epigenetic modifier can modulate histones through ubiquitination. In some cases, the histone epigenetic modifier can modulate histones by remodeling histone structure, e.g., via an ATP hydrolysis-dependent process.

In some embodiments, the transcriptional effector can be a gene epigenetic modifier (or a gene modifier). In some cases, a gene modifier can modulate genes through methylation (e.g., a gene methylation modifier, such as a DNA methyltransferase or DNMT). In some cases, a gene modifier can modulate genes through acetylation.

In some embodiments, the transcriptional effector can be derived from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the transcriptional effector can be derived from a histone lysine methyltransferase. Non-limiting examples of histone lysine methyltransferases include EZH subfamily, Non-SET subfamily, Other SET subfamily, PRDM subfamily, SET1 subfamily, SET2 subfamily, SUV39 subfamily, SYMD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

Non-limiting examples of the transcriptional effector that enhances expression or activity of the target gene can include, but are not limited to, transcriptional activators such as VP16, VP64, VP48, VP160, p65 subdomain (e.g., from NFkB), vp64-p65-rta fusion protein (VPR), and activation domain of EDLL and/or TAL activation domain (e.g., for activity in plants); histone lysine methyltransferases such as SETIA, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZMYST3, MORFMYST4, SRC1, ACTR, PI 60, CLOCK; and DNA demethylases such as Ten-Eleven Translocation (TET) dioxygenase 1 (TET1CD), TET1, DME, DML1, DML2, ROS1.

Non-limiting examples of the transcriptional effector that reduces expression or activity of the target gene can include, but are not limited to, transcriptional repressors such as the Kruppel associated box (KRAB or SKD); KOX1 repression domain; the Mad mSIN3 interaction domain (SID); the ERF repressor domain (ERD), the SRDX repression domain (e.g, for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARID1B/PLU-1, JARID 1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

Various aspects of the present disclosure provide for a plurality of heterologous genetic circuits that are individually activatable to modulate expression and/or activity levels of a plurality of distinct target genes in a sequential manner. In some embodiments, a first heterologous genetic circuit is activated to convert a plurality of cells from a first cell type to a second cell type and, subsequently, a second heterologous genetic circuit is activated to convert the plurality of cells from the second cell type to a target cell type.

In some embodiments, the activation of the second genetic circuit can be performed immediately following the activation of the first heterologous genetic circuit. Alternatively, the activation of the second genetic circuit can be performed at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 16 hours, at least about 20 hours, at least about 24 hours, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, at least about 1 year, or more subsequent to the activation of the first genetic circuit.

One or more target genes as disclosed herein can comprise a cell differentiation regulatory factor, a molecular function regulatory factor, a binding factor, a fusogenic factor, a protein folding chaperone, a protein tag, a RNA folding chaperone, a cell signaling factor, an immune response factor, a sensory receptor, a cell structural factor, a protein binding factor, a cargo receptor, a catalytic factor, or a small molecule sensor.

One or more target genes as disclosed herein can comprise a cell differentiation regulatory factor that comprises a growth factor, a transcription factor, a myogenic regulatory factor, an immune cell regulatory factor, a neuronal regulatory factor, a stem cell differentiation factor, a chondrogenic regulatory factor, an osteogenic regulatory factor, a senescence factor, a stemness factor (e.g., de-differentiation factor), etc.

In some cases, the one or more target genes (e.g., one or more myogenic regulatory factors) can comprise Msgn1, Pou5f1, Pax7, Myog, Myf5, Myf6, Myof, Srf, Ccnd2, Fgf2, Sox2, Tbx6, Tbxt, Ctnnb1, Mycl, Bcl212, Bcl211, Bcl2a1, Myod1, Bcl2, Tdgf1, Pax3, Chrd, Cond3, Hgf, Cond1, Foxm1, Myc, Tgfb1, Mdm2, Mcl1, Igf1, Mef2c, Mrf4, MyHC, MCK, Six1, Six4, Hdac4, Notch1, Ezh2, p21, Myh1, Myh2, Myh4, and/or Smad4.

In some cases, the one or more target genes can comprise T-box transcription factors (TBX genes). TBX transcription factors are involved in development. T-box proteins have relatively large DNA-binding domains. Non-limiting examples of TBX transcription factors can include TBX1, TBX2, TBX3, TBX4, TBX5, TBX6, TBX10, TBX15, TBX18, TBX19, TBX20, TBX21, TBX22, and TBXT (Brachyury protein).

In some cases, the target gene can comprise TBXT. TBXT, also known as T-box transcription factor T or brachyury protein, functions as a transcription factor in the T-box family of genes. TBXT has a role in defining the midline of a bilateral organism, helping to establish the anterior-posterior axis. It can also assist in defining the mesoderm during gastrulation.

In some cases, the target gene can comprise TBX6. TBX6, also known as T-box transcription factor 6, is involved in the segmentation of the paraxial mesoderm into somites.

In some cases, the one or more target genes can comprise a basic helix-loop-helix transcription factor (bHLH gene). bHLH transcription factors are involved in the regulation of the cell cycle and many other developmental processes. bHLH proteins have a basic helix-loop-helix protein structure. Non-limiting examples of bHLH transcription factors can include AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2.

In some cases, the target gene can comprise MSGN1. MSGN1, also known as mesogenin 1, is a Wnt-activated bHLH transcription factor which is involved in mesoderm formation and regulation of transcription by RNA polymerase II. MSGN1 can also be involved with somitogenesis.

In some cases, the one or more target genes can comprise a paired box transcription factor (PAX gene). PAX transcription factors are involved in tissue differentiation and development. Non-limiting examples of PAX transcription factors can include PAX1, PAX2, PAX3, PAX4, PAX5, PAX6, PAX7, PAX8, and PAX9.

In some cases, the one or more target genes can comprise PAX group 1, comprising PAX1 and/or PAX9. In some cases, the one or more target genes can comprise PAX group 2, comprising PAX2, PAX5 and/or PAX8. In some cases, the one or more target genes can comprise PAX group 3, comprising PAX3 and/or PAX7. In some cases, the one or more target genes can comprise PAX group 4, comprising PAX4 and/or PAX6.

In some cases, the target gene can comprise PAX3. PAX3, also known as paired-box transcription factor 3, is involved in ear, eye, and facial development. PAX3 can also contribute to tumor cell survival.

In some cases, the target gene can comprise PAX7. PAX7, also known as also known as paired-box transcription factor 7, is involved in myogenesis through the direction of postnatal renewal and propagation of myogenic satellite cells.

In some cases, the one or more target genes can comprise a cell de-differentiation factor (e.g., a Yamanaka factor). Non-limiting examples of dedifferentiation factors can include Oct3, Oct4, Sox2, Klf4, c-Myc, miR-302, miR-307, HERV-K, EEA, ZSCAN4, DUX4, OTX2, ABCE1, COL5A1, GAL4NT13, DUXA, DUXB, ARGFX, CPHX1, CPHX2, TPRX1, DPPA3, NASP, ATP2B1, NFAT, and/or CAMKII.

In some cases, the target gene can comprise OCT4. OCT4, also known as octamer-binding transcription factor 4 or POU5F1, is a transcription factor in the POU family. OCT4 is involved in the self-renewal of undifferentiated embryonic stem cells and is a commonly used marker for undifferentiated cells.

In some embodiments, the plurality of gate units can comprise a first gate unit and a second gate unit. In some cases, the heterologous genetic circuit can be configured (or preprogrammed) such that (i) the first gate unit is activated to target and modulate a first target gene (e.g., a first target endogenous gene) and (ii) the second gate unit is activated to target and modulate a second target gene (e.g., a second target endogenous gene) in a cell. The first gate unit and the second gate unit can be activated at different times. The heterologous genetic circuit can be configured such that activation of the first gate unit occurs prior to (or alternatively subsequent to) activation of the second unit. Alternatively, the first gate unit and the second gate unit can be activated substantially at the same time. Accordingly, the heterologous genetic circuit can be configured such that modulation of the first target gene occurs prior to (or alternatively subsequent to) modulation of the second target gene. Alternatively, the first target gene and the second target gene can be modulated substantially at the same time.

In some cases, the first target gene can comprise a member selected from the group consisting of a T-box transcription factor (TBX), a basic helix-loop-helix transcription factor (bHLH), and a Paired box transcription factor (PAX), and the second target gene can comprise a different member selected form the group consisting of the TBX, the bHLH, and the PAX. For example, the first target gene can be TBX, and the second target gene can be bHLH and/or PAX. In another example, the first target gene can be bHLH, and the second target gene can be TBX and/or PAX. In another example, the first target gene can be PAX and the second target gene can be TBX and/or bHLH.

In some cases, the first target gene can comprise a first species of TBX, and the second target gene can comprise a second species of TBX that is different from the first species of TBX. The two different species of TBX can be selected from the group consisting of TBX1, TBX2, TBX3, TBX4, TBX5, TBX6, TBX10, TBX15, TBX18, TBX19, TBX20, TBX21, TBX22, and TBXT. For example, the first species can be TBX1, and the second species can be selected from the group consisting of TBX2, TBX3, TBX4, TBX5, TBX6, TBX10, TBX15, TBX18, TBX19, TBX20, TBX21, TBX22, and TBXT. In another example, the first species can be TBX2, and the second species can be selected from the group consisting of TBX1, TBX3, TBX4, TBX5, TBX6, TBX10, TBX15, TBX18, TBX19, TBX20, TBX21, TBX22, and TBXT. In another example, the first species can be TBX3, and the second species can be selected from the group consisting of TBX1, TBX2, TBX4, TBX5, TBX6, TBX10, TBX15, TBX18, TBX19, TBX20, TBX21, TBX22, and TBXT. In another example, the first species can be TBX4, and the second species can be selected from the group consisting of TBX1, TBX2, TBX3, TBX5, TBX6, TBX10, TBX15, TBX18, TBX19, TBX20, TBX21, TBX22, and TBXT. In another example, the first species can be TBX5, and the second species can be selected from the group consisting of TBX1, TBX2, TBX3, TBX4, TBX6, TBX10, TBX15, TBX18, TBX19, TBX20, TBX21, TBX22, and TBXT. In another example, the first species can be TBX6, and the second species can be selected from the group consisting of TBX1, TBX2, TBX3, TBX4, TBX5, TBX10, TBX15, TBX18, TBX19, TBX20, TBX21, TBX22, and TBXT. In another example, the first species can be TBX10, and the second species can be selected from the group consisting of TBX1, TBX2, TBX3, TBX4, TBX5, TBX6, TBX15, TBX18, TBX19, TBX20, TBX21, TBX22, and TBXT. In another example, the first species can be TBX15, and the second species can be selected from the group consisting of TBX1, TBX2, TBX3, TBX4, TBX5, TBX6, TBX10, TBX18, TBX19, TBX20, TBX21, TBX22, and TBXT. In another example, the first species can be TBX18, and the second species can be selected from the group consisting of TBX1, TBX2, TBX3, TBX4, TBX5, TBX6, TBX10, TBX15, TBX19, TBX20, TBX21, TBX22, and TBXT. In another example, the first species can be TBX19, and the second species can be selected from the group consisting of TBX1, TBX2, TBX3, TBX4, TBX5, TBX6, TBX10, TBX15, TBX18, TBX20, TBX21, TBX22, and TBXT. In another example, the first species can be TBX20, and the second species can be selected from the group consisting of TBX1, TBX2, TBX3, TBX4, TBX5, TBX6, TBX10, TBX15, TBX18, TBX19, TBX21, TBX22, and TBXT. In another example, the first species can be TBX21, and the second species can be selected from the group consisting of TBX1, TBX2, TBX3, TBX4, TBX5, TBX6, TBX10, TBX15, TBX18, TBX19, TBX20, TBX22, and TBXT. In another example, the first species can be TBX22, and the second species can be selected from the group consisting of TBX1, TBX2, TBX3, TBX4, TBX5, TBX6, TBX10, TBX15, TBX18, TBX19, TBX20, TBX21, and TBXT. In another example, the first species can be TBXT, and the second species can be selected from the group consisting of TBX1, TBX2, TBX3, TBX4, TBX5, TBX6, TBX10, TBX15, TBX18, TBX19, TBX20, TBX21, and TBX22. For instance, the first species can be TBXT and the second species can be TBX6.

In some cases, the first target gene can comprise at least one cell de-differentiation factor as provided herein (e.g., Oct4, Sox2, Klf4, c-Myc, etc.), and the second target gene can comprise at least one tissue-specific differentiation factor as provided herein (e.g., TBX, bHLH, and/or the PAX). The at least one cell de-differentiation factor and the at least one tissue-specific differentiation factor are different factors (e.g., different proteins, different polynucleotide molecules, etc.).

In some cases, use of the heterologous genetic circuit as disclosed herein can be used to differentiate muscle stem cells (MuSCs) into muscle cells whereby at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% of the resulting cells generated by using the heterologous genetic circuit as disclosed herein are the target cell type.

In some cases, use of the heterologous genetic circuit as disclosed herein can be used to differentiate muscle stem cells (MuSCs) into muscle cells, e.g., in absence of one, two, or all of feeder cells, serum, and exogenous growth factors. Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹⁵, at least about 2×10¹⁵, at least about 5×10¹⁵, or more T cells from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or more muscle cells.

In some cases, use of the heterologous genetic circuit as disclosed herein can be used to differentiate pluripotent stem cells (PSCs, such as induced PSCs or iPSCs) into muscle cells, e.g., in absence of one, two, or all of feeder cells, serum, and exogenous growth factors. Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹⁵, at least about 2×10¹⁵, at least about 5×10¹⁵, or more T cells from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or more muscle cells.

Such generation of muscle cells by using the heterologous genetic circuit as disclosed herein can be achieved within the span of at most about 60 days, at most about 55 days, at most about 50 days, at most about 45 days, at most about 40 days, at most about 35 days, at most about 30 days, at most about 25 days, at most about 20 days, at most about 15 days, at most about 10 days, at most about 7 days, at most about 6 days, at most about 5 days, at most about 4 days, at most about 3 days, at most about 2 days, at most about 1 day, or less.

In some cases, the muscle cells generated through this method have more nuclei per myotube as compared to muscle cells obtained via directed differentiation. The muscle cells generated using the provided methods can have at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 more nuclei per myotube than muscle cells obtained via directed differentiation. Alternatively, or in addition to, the muscle generated through this method may have equivalent numbers of nuclei as compared to muscle cells obtained via directed differentiation.

In some cases, the muscle cells or muscle progenitor cells generated through this method exhibit higher expression levels of two or more muscle stem cell markers as compared to control PSCs. Non-limiting examples of muscle stem cell markers can include CD271, ERBB3, CD54, ITGA9, or SDC2. Myogenic progenitor cells can be identified using SDC2, ITGA9, CD54 and myogenic progenitor surface markers. Alternatively, myogenic progenitor cells can be identified using ERBB3, CD271 (NGFR) and myogenic progenitor surface markers.

In some cases, the muscle cells or muscle progenitor cells generated through this method exhibit at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% higher expression levels or more as compared to control PSCs.

In some cases, the muscle cells or muscle progenitor cells generated through this method exhibit at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold higher expression levels or more as compared to control PSCs. In some cases, expression levels of muscle stem cell markers can be measured using methods such as, but not limited to, RT-PCR, Western blotting, Northern blotting, protein staining, mRNA staining, and RNA-sequencing.

In some cases, expression levels can be measured at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 28 hours, at least about 32 hours, at least about 36 hours, at least about 40 hours, at least about 44 hours, at least about 48 hours, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days or more days after the introduction of a genetic circuit.

In some cases, the muscle cells or muscle progenitor cells generated through this method can be myoblasts. Alternatively, the muscle cells or muscle progenitor cells generated through this method can be cells other than myoblasts (e.g., muscle satellite cells). In some cases, the progenitor cells generated can be substantially mitotically dormant. Alternatively, the progenitor cells generated can be substantially mitotically active.

In some cases, the progenitor cells generated do not express Myf5. In some cases, the progenitor cells generated express less Myf5 as compared to control myoblast cells. In some cases, the generated progenitor cells express at least about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% less Myf5 as compared to control myoblast cells.

In some cases, the progenitor cells generated express at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold less Myf5 as compared to control myoblast cells.

In some cases, the progenitor cells generated do not express MyoD. In some cases, the progenitor cells generated express less MyoD as compared to control myoblast cells. In some cases, the generated progenitor cells express at least about at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% less MyoD as compared to control myoblast cells.

In some cases, the progenitor cells generated express at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold less MyoD as compared to control myoblast cells.

In some cases, a first gate unit can be configured to reduce expression and/or activity levels of one or more target genes. In some cases, a first gate unit can be configured to enhance expression and/or activity levels of one or more target genes. In some cases, a first gate unit can be configured to maintain expression and/or activity levels of one or more target genes.

In some cases, modulation of a first target gene can occur prior to modulation of a second target gene. In some cases, modulation of a first target gene can occur subsequent to modulation of a second target gene. In some cases, modulation of a first target gene can occur at about the same time as modulation of a second target gene.

In some cases, use of the heterologous genetic circuit can induce cells to differentiate into the cell type of interest in absence of growth factors, serum (fetal bovine serum, human serum AB, etc.), or other exogenous cell differentiation regulatory factors or mediums. Serum can comprise liquid fractions of clotted blood, including nutritional and macromolecular factors essential for cell growth.

Alternatively, use of the heterologous genetic circuit can induce cells to differentiate into the cell type of interest using a reduced amount of serum and/or growth factors (e.g., reduced by at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or substantially free of serum). Reduced amounts of serum can allow for more consistency across experiments or batches of cells, increased growth and/or productivity of differentiated cells, better control over physiological responsiveness, and reduced risk around contamination by serum-born agents in cell culture.

In some cases, use of the heterologous genetic circuit in the stem cells (e.g., iSPCs, MSCs, MuSCs) can induce the MSCs to differentiate into muscle cells in absence of growth factors, serum (fetal bovine serum, human serum AB, etc.), or other exogenous cell differentiation regulatory factors or mediums. In some cases, use of the heterologous genetic circuit as disclosed herein can be used to differentiate the stem cells into muscle cells in the absence of one or both growth factors and serum. The resulting muscle cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population.

In some cases, conversion from one cell type (e.g., PSCs, MSCs or myoprogenitor cells) to another cell type (e.g., muscle cells) using a heterologous genetic circuit can result in the target cell type. Alternatively, conversion from one cell type (e.g., PSCs, MSCs or myoprogenitor cells) to another cell type (e.g., muscle cells) using a heterologous genetic circuit can result in an intermediate cell type. An intermediate cell type can undergo a second conversion using a second genetic circuit to result in the target cell type.

The conversion of a cell from one cell type to another can comprise the regulation of a plurality of target genes. For example, the conversion can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The conversion can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic circuit and/or (ii) the activating moiety of the heterologous genetic circuit.

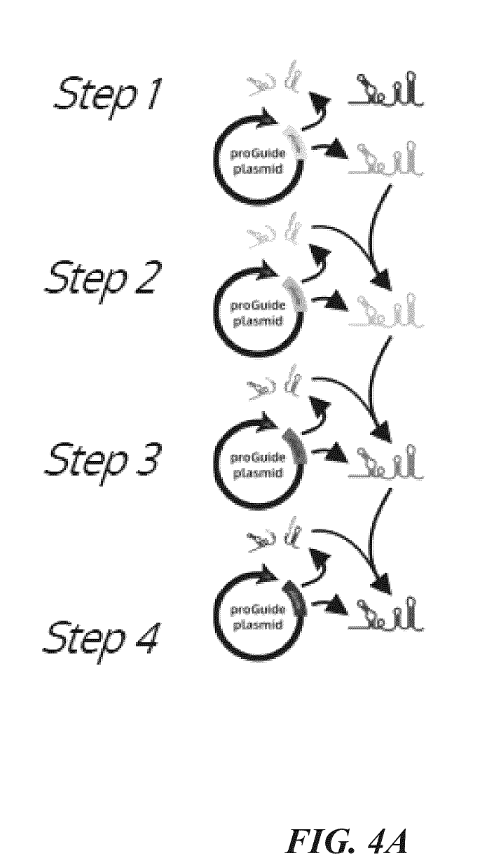

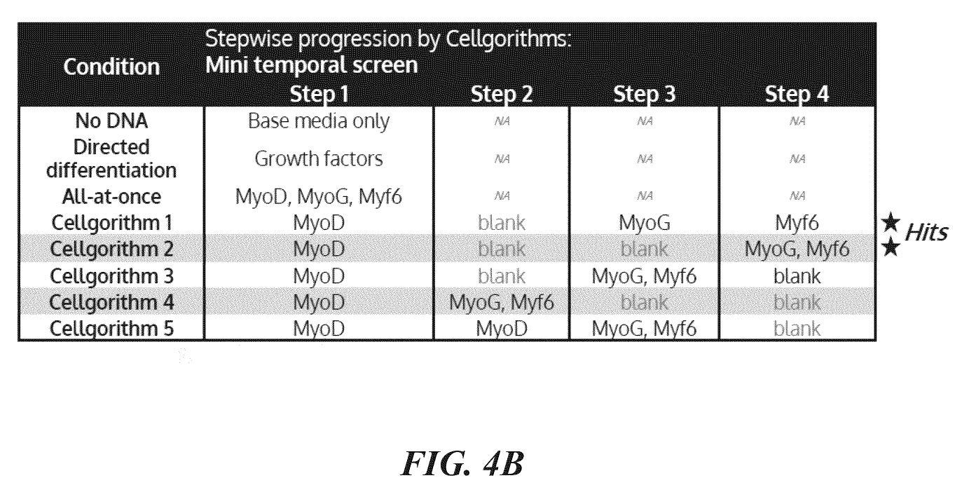

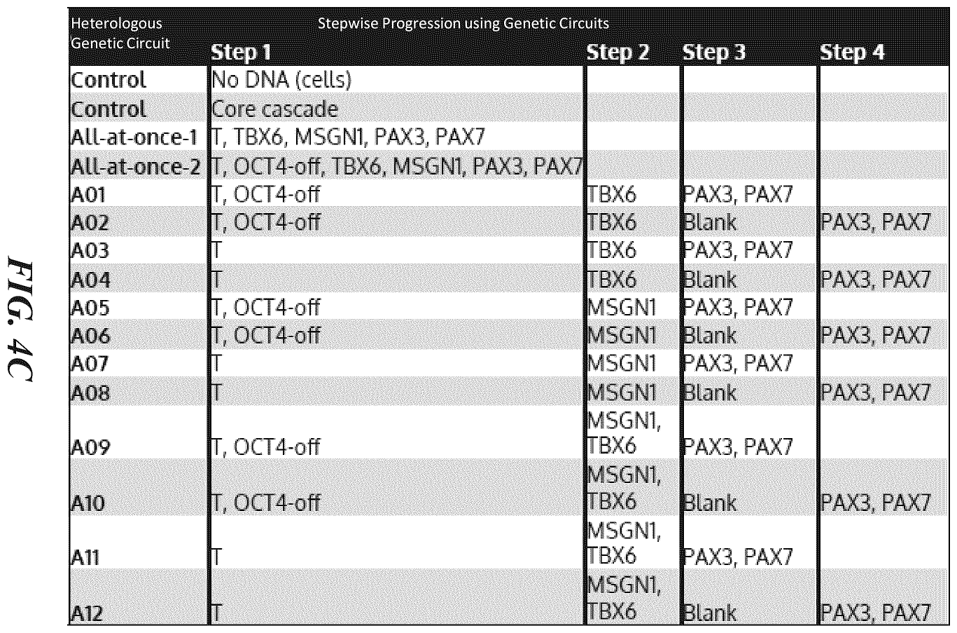

As demonstrated in FIG. 4C, various heterologous genetic circuits can be designed to modulate expression or activity levels of a plurality of genes (e.g., a plurality of endogenous genes) in a cell over a plurality of different time points. For example, heterologous genetic circuit #10 can be designed to (i) activate expression level of TBXT (denoted as T) at time point 1 (denoted as step 1), (ii) subsequently activate expression levels of MSGN1 and TBX6 at a time point after step 1 (denoted as step 2), (iii) subsequently activate expression levels of PAX3 and PAX7 at a time point after step 2 (denoted as step 4). For comparison, a control heterologous genetic circuit (denoted as All-at-once 1) can be designed to simultaneously activate the same target endogenous genes from the heterologous genetic circuit #10.

Activating a heterologous genetic circuit in a cell as disclosed herein can modulate expression or activity levels of a plurality of genes over a plurality of different time points, to effect conversion of the cell into a different cell type (e.g., stem cells into tissue-specific progenitor cells, etc.). A rate of such cell type conversion by use of the heterologous genetic circuit can be greater than a rate of the cell type conversion by use of the control heterologous genetic circuit (e.g., for simultaneous activation of multiple target genes) by at least or up to about 1 percent (%), at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 60%, at least or up to about 70%, at least or up to about 80%, at least or up to about 90%, or at least or up to about 95%.

A cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, an secretory cell, a blood cell, a germ cell, a nurse cell, a storage cell, an enteroendocrine cell, a pituitary cell, a neurosecretory cell, a duct cell, an odontoblast, a cementoblast, a glial cell, or an interstitial cell.

Non-limiting examples of such cell can include lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and

### Interstitial Kidney Cells.

In an aspect, the present disclosure provides for systems and methods that can convert a plurality of pluripotent stem cells (PSCs) into a plurality of tissue-specific progenitor cells.

A pluripotent stem cell can comprise an induced pluripotent stem cell (iPSC), or an embryonic stem cell (ESC). A tissue-specific progenitor cells can comprise a mesenchymal stem cell (MSC), a hematopoietic stem cell (HSC), a myeloid progenitor cell, a muscle stem cell, a myoprogenitor cell, a neural stem cell, an epithelial stem cell, an epidermal stem cell, a mammary stem cell, an intestinal stem cell, a neural crest stem cell, or a testicular stem cell.

Various aspects of the present disclosure provide engineered cells, or any further engineered variant thereof, that are programmed to induce a desired expression and/or activity level (or profile thereof) of one or more target genes in a cell.

In some embodiments, the engineered cell (e.g., the engineered muscle cell) of the present disclosure can be generated from an isolated stem cell (e.g., isolated MSCs or MuSCs). The heterologous genetic circuit and/or its components (e.g. gate units, gate moieties, activating moieties, etc.), as disclosed herein, can be introduced during any stage (or cellular state) between and including (a) the isolated stem cell and (b) the differentiated muscle cell state thereof (e.g. a terminally differentiated muscle cell state, such as a skeletal muscle cell.

The engineered cell (e.g., the engineered muscle cell) of the present disclosure can be used (e.g., administered) to treat a subject in need thereof. The subject can have or can be suspected of having a condition, such as a disease (e.g., cancer). A cell (e.g., a stem cell or a differentiated cell) can be obtained from the subject and such cell can be cultured ex vivo and genetically modified to generate any subject engineered cell (e.g. a muscle cell) as disclosed herein. Subsequently, the engineered immune cell can be administered to the subject for adaptive immunotherapy. Thus, the engineered cell can be autologous to the subject in need thereof. Alternatively, the engineered cell can be allogeneic to the subject (e.g., allogeneic stem cell transplantation, allogeneic adoptive immunotherapy, etc.).

The engineered cells, or any further engineered variant thereof, as disclosed herein can be administered to the subject prior to, concurrently with, or subsequent to activation of the heterologous genetic circuit(s) in the engineered stem cells, or any further engineered variant thereof. For example, the engineered cells, or any further engineered variant thereof, can be activated subsequent to being administered into the subject, e.g., by administering to the subject an activator of the heterologous genetic circuit(s).

The subject can be treated (e.g., administered with) a population of engineered cells (e.g., engineered muscle cells), or any further engineered variant thereof, of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses. Alternatively, or in addition to, the subject can be treated (e.g., administered with) a population of engineered cells (e.g., engineered T cells), or any further engineered variant thereof, of the present disclosure for at least about 1 week, at least about 2 weeks, at least about 3 weeks, at least about 4 weeks, at least about 5 weeks, at least about 6 weeks, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, at least about 30 years, at least about 40 years, at least about 50 years, at least about 60 years, at least about 70 years, at least about 80 years, at least about 90 years, or at least about 100 years.

Any one of the methods disclosed herein can be utilized to treat a target cell, a target tissue, a target condition, or a target disease of a subject.

The disease can be a neuromuscular disease or disorder, including, but are not limited to, muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy), motor neuron diseases (e.g. amyotrophic lateral sclerosis (ALS), Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphogly cerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenita paramyotonia congenita central core disease nemaline myopathy myotubular myopathy periodic paralysis).

The disease can be specifically a muscular disease or disorder, such as myotonic dystrophy type 1 (DM1), Duchenne muscular dystrophy (DMD), Becker muscular dystrophy (BMD), limb girdle muscular dystrophy type 1B (LGMD1B), LMNA-linked dilated cardiomyopathy (DCM), Hutchinson-Gilford progeria syndrome (HGPS), Familial partial lipodystrophy type 2 (FPLD2), spinal muscular atrophy (SMA), or amyotrophic lateral sclerosis (ALS).

Non-limiting examples of the target tissue can include cells, for example muscle cells, can be obtained from a subject. Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

The target disease of the subject can be cancer or tumor. Non-limiting examples of cancer can include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

The present disclosure also provides a composition comprising the engineered genetic circuit(s) as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered cells, or any further engineered variant thereof. Alternatively or in addition to, the activator(s) can be in a different and separate composition from the engineered cells, or any further engineered variant thereof.

In some cases, the engineered progenitor cells disclosed herein can exhibit (i) comparable or enhanced regenerative capacity; (ii) comparable or enhanced in vitro expression; (iii) comparable or enhanced genetic editing capabilities; (iv) comparable or enhanced immunotolerance; (v) comparable or shorter manufacturing timelines; (vi) comparable or fewer growth factor or culturing requirements; and/or (vii) comparable or enhanced safety, as compared to a control progenitor cell.

The control progenitor cell can be generated by any method comprising expansion of a progenitor cell (e.g., muscle satellite cell) isolated from a tissue, directed iPSC differentiation (e.g., using exogenous growth factors), and/or transgenic iPSC differentiation (e.g., viral transduction of heterologous genes).

In some cases, the tissue-specific progenitor cells can be stored in a receptacle (e.g., a sterilized vial). In some cases, the tissue-specific progenitor cells are stored at a temperature of at most about 10° C., at most about 5° C., at most about 4° C., at most about 0° C., at most about −5° C., at most about −10° C., at most about −20° C., at most about −30° C., at most about −40° C., at most about −50° C., at most about −60° C., at most about −70° C., at most about −80° C., at most about −90° C., at most about −100° C., at most about −110° C., at most about −120° C., at most about −130° C., at most about −140° C., at most about −150° C., at most about −160° C., at most about −170° C., at most about −180° C., at most about −190° C., at most about −200° C., or colder.

The present disclosure also provides a composition comprising the engineered genetic modulators and/or the engineered genetic circuits as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered genetic modulators and/or the engineered genetic circuits. Alternatively or in addition to, the activator(s) can be in a different and separate composition from the engineered genetic modulators and/or the engineered genetic circuits.

### Pharmaceutical Compositions

In some cases, methods disclosed herein comprise administering at least one of the tissue-specific progenitor cells to a subject in need thereof. A subject can be an animal. A subject can be a mammal (e.g., a primate, a horse, a cat, a dog, a cow, a pig, a sheep, a goat, a mouse, a rabbit, a rat, a guinea pig). A subject can be a human subject.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

### EXAMPLES

### Example 1: Differentiation of Myogenic Progenitor Cells

Tissue-specific cells (e.g., myoprogenitor cells) can be generated from less-differentiated cells (e.g., stem cells, such as iPSCs) by the systems and methods of the present disclosure.

### A. Generation of Myoprogenitor Cells.

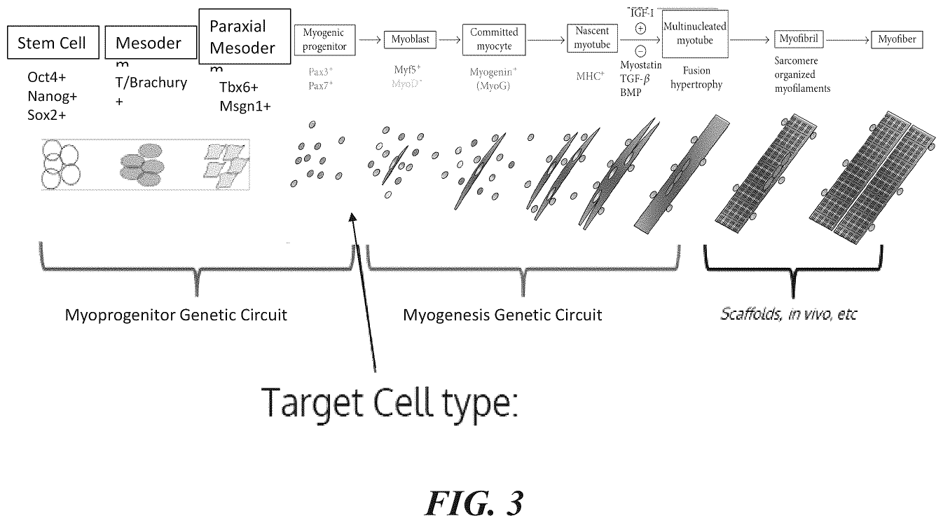

In this example, stem cells (e.g. iPSCs) were formed into tissue-specific cells (e.g. myoprogenitor cells) using heterologous genetic circuits. Differentiation of a stem cell (e.g., iPSC) to a tissue-specific cell (e.g. myoprogenitor cell) can be a complex process requiring turning on a plurality of endogenous genes at different time points, while turning off a plurality of endogenous genes at different time points. See FIG. 3 for examples of different endogenous genes that are induced for expression at different stages of a differentiation of a stem cell to a myoprogenitor cell, then to a myoblast, then into a muscle tissue. Thus, one or more heterologous genetic circuits as disclosed herein can be utilized to automatically promote regulation of such cascade of different endogenous gene expressions. In some cases, each heterologous genetic circuit can be configured to regulate expression levels of a plurality of genes at a plurality of different time points upon a single activation of such heterologous genetic circuit.

The stem cells (e.g. iPSCs) were transiently transfected with plasmid DNAs encoding one of the heterologous genetic circuits as described in FIG. 4C, e.g., targeting TBX6, TBXT (denoted as T), MSGN1, PAX3, PAX7, and OCT4. All genes targeted were activated with the exception of OCT4, which was targeted for deactivation. The stem cells (e.g. iPSCs) were cultured in simple media (e.g., no serum). Flow cytometry was used to analyze for Pax3/Pax7 double positive cells, which indicated the formation of tissue-specific (e.g. myogenic progenitor cells).

For instance, heterologous genetic circuit vectors were purified as single plasmids in an endotoxin free manner and then pooled. Each pool contained sgRNAs for step 1 and gate moiety vectors for steps 2, 3, and 4 of each heterologous genetic circuit as shown in FIG. 4C. The gate moiety vectors were pooled at an equal ratio, and expression plasmid (e.g. Cas9-VPR) was added at a 1:1 ratio to the total mass of the gate moiety vectors, as well as GFP expression plasmid to mark transfected cells. After pooling the DNA, human stem cells (e.g. iPSC) maintained in E8 media (vitronectin coated flasks) were lifted using accutase and pelleted (e.g. to 30,000 cells per well of 96 well plate). To make the transfection mix, the heterologous genetic circuit vector pool (e.g. 130 ng) was added to serum free DMEM (e.g. 20 microliters) followed by lipofection reagent (e.g. 0.3 microliters). After a period of time (e.g. 15 minutes), the transfection mix was used to resuspend the stem cell (e.g. iPSC) pellet, which was then re-plated into E6 base media containing ITS. The cells were grown for days (e.g. five days) with a media change on day 3. On day 5, the cells were lifted using TrypLE, pelleted, and incubated with antibodies against, CD54, SDC2, ITGA9. Cells were then run through a flow cytometer and the percentage of each marker was quantified using single live cells as the parental gate.

### B. Characterization of Myoprogenitor Cells Generated by a Heterologous Genetic Circuit

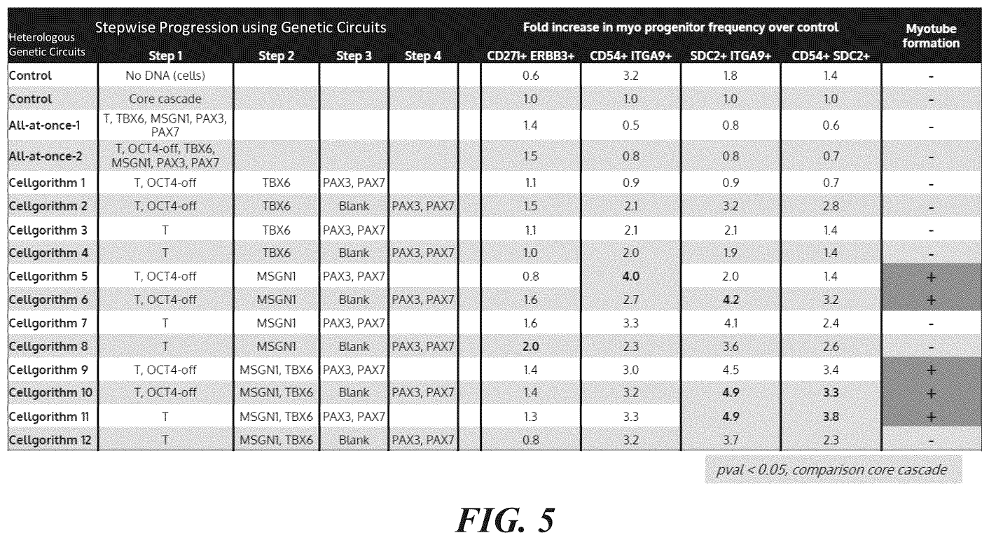

FIG. 5 shows that transient plasmid delivery of the heterologous genetic circuits induced double positive myogenic progenitor cell markers to appear after five days. Myotube formation was see in five of the genetic circuits tested.

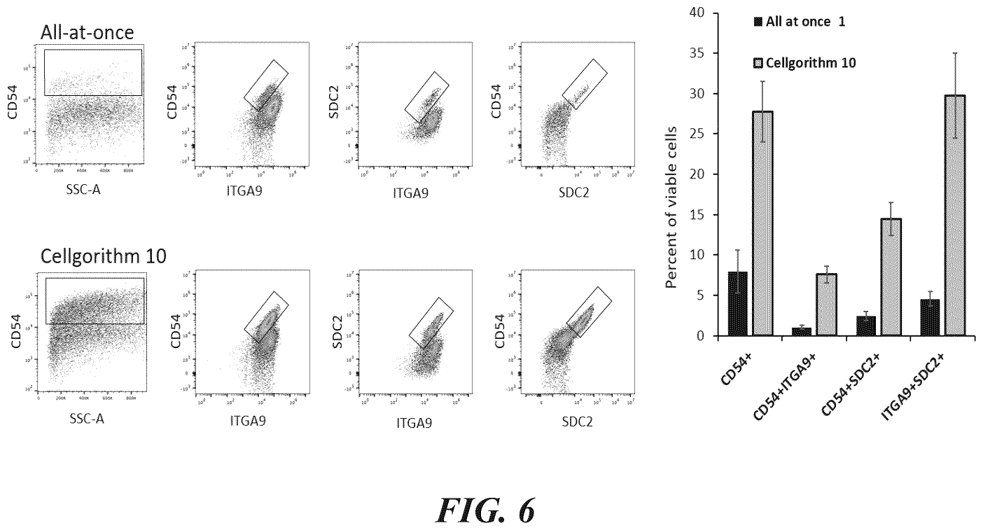

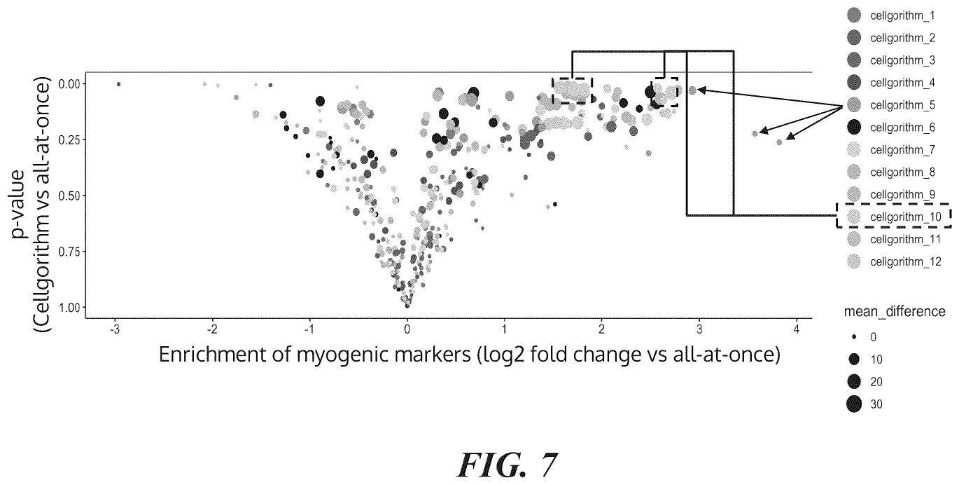

As shown in FIG. 6, flow analysis revealed that at least one of the heterologous genetic circuits provided in FIG. 4C yield at least about 30% iPSC-to-myogenic progenitor cell (e.g., ITGA9+ and SDC2+ cells) conversion, in 5 days. This conversion rate was greater than a control population of iPSCs that were treated by activating the same endogenous genes, but all at the same time. For example, the heterologous genetic circuit 10 (e.g., Cellgorithm 10) from FIG. 4C activated 5 genes throughout the course of multiple time points (e.g., 4 different time points), and the resulting stem cell to tissue-specific cell (e.g. iPSC-to-myogenic progenitor cell) conversion rate was at least about 6 times greater than that resulting from activating the same 5 genes at the same time. Separately, cells treated with various heterologous genetic circuits from FIG. 4C were analyzed (e.g., via flow) for the positive markers of myoprogenitor cells (e.g., SDC2+CD54+, SDC2+ITAG9+, CD271+EBB3+, CD54+ITGA9+, etc.), then plotted in a volcano plot, as shown in FIG. 7. The volcano plot was generated to compare the efficiency of each heterologous genetic circuit in terms of (i) statistical significance (p-value) as compared to the all-at-once control circuit that activated the target endogenous genes at once, versus (ii) magnitude of change (fold change) of the positive markers of myoprogenitor cells as compared to the all-at-once control circuit. Each axis was value was in comparison to the all-at-once control circuit, in order to emphasize the importance of the order in which the target endogenous genes are manipulated via the heterologous genetic circuits disclosed herein. As indicated by the dotted boxes in FIG. 7, at least the heterologous genetic circuit 10 (e.g, Cellgorithm 10) from FIG. 4C resulted in one of the highest fold change in expression of the myoprogenitor cell markers (x-axis) along with one of the highest p-values (y-axis), suggesting that this heterologous genetic circuit efficiently and rapidly produced myogenic progenitors (e.g., in 5 days). Separately, some of the data from a cell sample treated with heterologous genetic circuit #5 (or Cellgorithm 5) is indicated by the three arrows, as a comparison to show a heterologous genetic circuit design that was able to induce a higher fold change in the multiple myoprogenitor cell marker expressions but at a lower p-value, as compared to the heterologous genetic circuit 10.

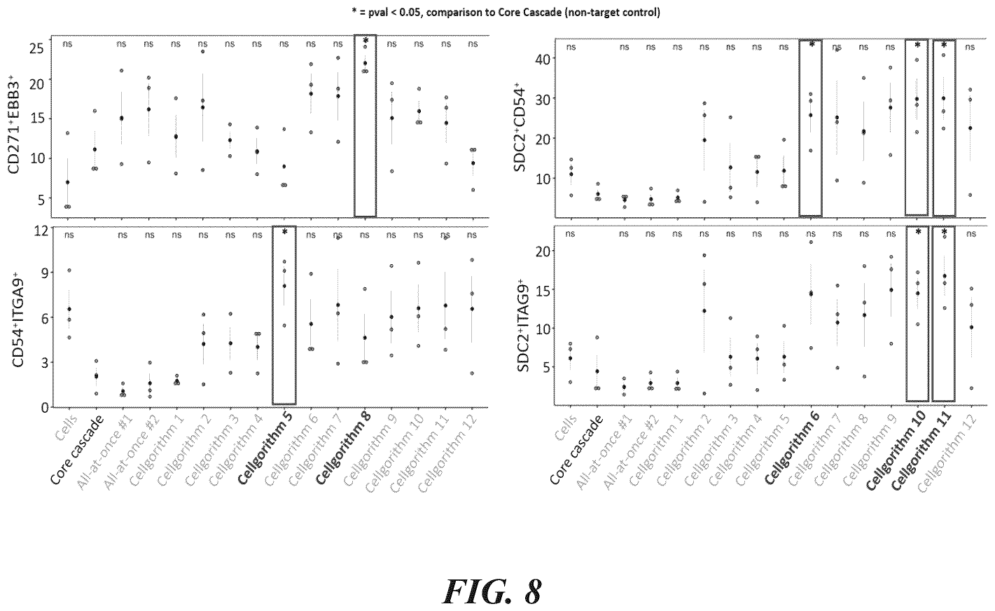

FIG. 8 shows representative data utilized in the volcano plot of FIG. 7. In each plot of FIG. 8, the y-axis represents proportion of each cell sample that express the indicated myoprogenitor cell markers (e.g., SDC2+CD54+, SDC2+ITAG9+, or CD271+EBB3+, CD54+ITGA9+) upon treatment with one of the heterologous genetic circuits from FIG. 4C. The x-axis indicates which heterologous genetic circuit was utilized for each cell sample. The plots in FIG. 8 show that various heterologous genetic circuits (e.g., Cellgorithm 10, Cellgorithm 11, etc.) can generate myoprogenitor cells as indicated by multiple myoprogenitor cell marker panels.

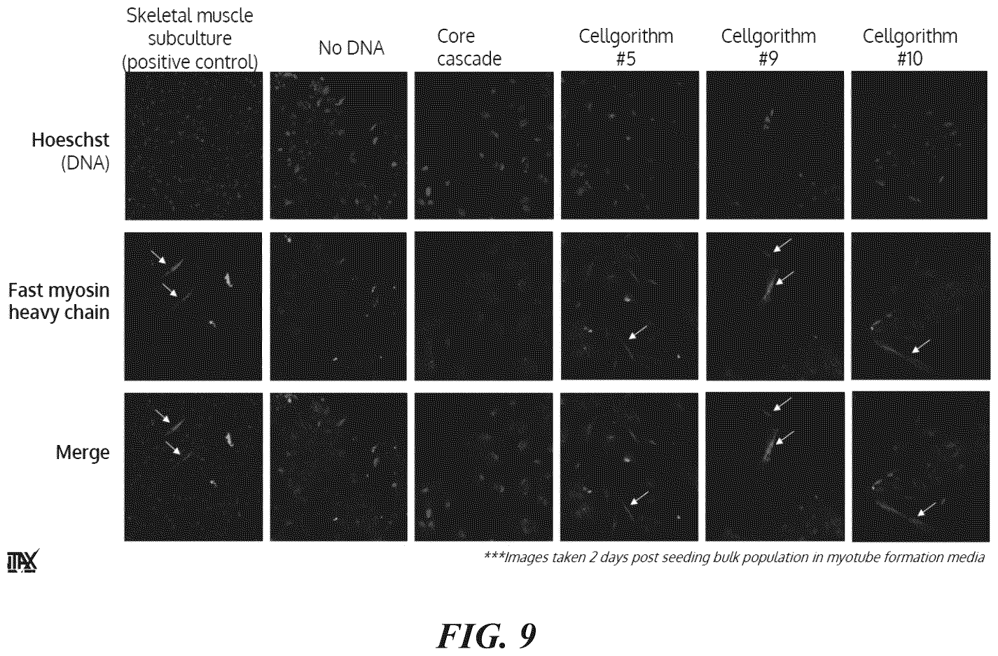

Furthermore, myogenic progenitor cells selected using flow cytometry were placed in myotube formation media to encourage further myogenesis. FIG. 9 shows myoprogenitor cell formation using heterologous genetic circuits (e.g., Cellgorithm #5, #9, and #10 from FIG. 5). Arrows point to the formation of myotubes.

### Example 2: Implantation and Engraftment of Myogenic Progenitor Cells

Tissue-specific cells (e.g., myoprogenitor cells) as prepared by the systems and methods of the present disclosure can be administered to a subject in need thereof (e.g., injected into a muscle tissue, such as skeletal muscle or heart), to treat a neuromuscular disorder or a muscular disorder of a subject.

### A. Generation of Myoprogenitor Cells.

Stem cells (e.g., iPSCs) can be transduced or transfected (e.g., transiently transfected) with one or more heterologous genes (e.g., plasmid DNAs) encoding a at least one heterologous genetic circuit, such as, for example, one of the respective heterologous genetic circuit as shown in FIG. 4C, in accordance with the methods described in Example 1, in order to generate myoprogenitor cells.

### B. In Vivo Administration of Myoprogenitor Cells

Upon generation of myoprogenitor cells by systems and methods of the present disclosure, the myoprogenitor cells (e.g., CD54+ cells) can be purified using anti-CD54 antibody. Cells can be concentrated and resuspended in a buffer (e.g., PBS), and then be administered to mice via intramuscular injection directly into the site of wound or interest. After 8 to 10 weeks, the mice can be sacrificed, and the muscle tissue surrounding the area of injection can be sectioned. Sections can be immunostained for human dystrophin and human lamin A/C to confirm the engraftment of the ex vivo-generated myoprogenitor cells.

An additional protocol for myoprogenitor cell transplantation is provided herein. Upon generation as described herein, the myoprogenitor cells can be suspended in myogenic cell media (e.g., F10/DMEM (50/50)+1 5% FBS+2.5 ng/ml bFGF). These cells can be transplanted into a target site in a muscle tissue with or without further expansion. For expansion, the myoprogenitor cells can be plated into tissue culture wells containing hydrogels (flat or patterned) or thin gel coated plastic (flat or patterned) as sparse cultures (e.g. 1000-2000 cells/well of a 24 well size plate), and cultured while replacing media every 3 days. On the day of transplantation, NOD/SCID mice can be anesthetized by injection (e.g. intraperitoneal) of anesthetics (e.g. Ketamine (2.4 mg/mouse) and Xylazine (240 g/mouse)) at an appropriate dosage and hindlimb irradiated as previously described (A. Sacco et al. (2008) Nature 456, 502). The generated myoprogenitor cells can be counted and resuspended in media (e.g. 2.5% goat serum/1 mM EDTA in PBS), and subsequently injected intramuscularly (e.g. into the tibialis anterior (TA) muscles) into recipient mice. Optionally, to test engraftment of the generated myoprogenitor cells in an injured tissue, animals can be anesthetized with isofluorane and a single injection of notexin can be injected into recipient animal TA muscles.

Engraftment (e.g., differentiation and integration into the local muscle tissue) of the transplanted myoprogenitor cells can be visualized by various methods. For example, the myoprogenitor cells can be engineered to express a heterologous marker (e.g., fluorescent proteins, such as green fluorescent protein) that is not present in the transplanted animal. Alternatively or in addition to, the myoprogenitor cells can be allogeneic to the animal, such that any myoblasts or myotubes that are differentiated from the myoprogenitor cells upon the transplantation can be identified (e.g., immunostaining) by an antigen that is not found in the transplanted animal.

### Example 3: Myogenic Progenitor Mouse Model

Methods and systems of the present disclosure can be utilized to generate tissue specific progenitor cells for repair or regeneration of the tissue. In some cases, skeletal tissue-specific progenitor cells (e.g., myogenic progenitors) can be generated accordingly to repair or regeneration of a skeletal tissue (e.g., muscle tissue).

In some cases, myogenic progenitor cells generated in accordance with the methods and systems provided herein can be used to repair skeletal muscle that has been damaged or impaired by disease (or suspected thereof), e.g., Duchenne muscular dystrophy. Such myogenic progenitors provided herein can be used in a comparable or improved manner as compared to other types of myogenic progenitors, e.g., natural myogenic progenitors isolated from an existing skeletal muscle, or those differentiated from pluripotent stem cells via other methods that require changing of exogenous growth factors and other small molecules over a period of time in a cell culture medium (e.g., directed differentiation). Regardless of their origin, myogenic progenitor cells can be evaluated and used in vivo by injecting them directly (e.g. into a muscle) at the site of intended repair or growth of new muscle fibers. Myogenic progenitor cells can be phenotypically characterized based on gene expression and cell surface marker characteristics, and can be functionally characterized for their ability to form multinucleated myofibers in vitro. However, the in vitro characterizations provide substantially less information compared to in vivo functional testing in animal models.

To evaluate function of myogenic progenitor cells, animal models use an injection (e.g. into a muscle) of the cells that has been damaged by a genetic disease state and/or a physically- or chemically-induced injury. Accordingly, an example protocol for injecting cells into an injury (e.g. cardiotoxin-induced) in the mouse muscle (e.g. tibialis anterior) is provided herein. An important criterion for assessing the therapeutic potential of the myogenic progenitor cells can include assessing whether repair and regrowth of skeletal muscle is associated with engraftment of the injected cells into new muscle tissue. Repair of damaged muscle can occur even without incorporation of the injected cells into the muscle, therefore engraftment (and not repair) is the critical outcome measured for most animal models. Engraftment is determined by detecting a marker unique to human cells in the mouse muscle after it has been removed, embedded in a cryomold, sectioned onto slides, and stained by indirect immunofluorescence using antibodies specific for the human antigen. Alternatively or in addition to being able to detect the presence of human cells in the mouse muscle, it is also important to determine if those cells have formed myofibers and have been incorporated into the existing mouse muscle. Two human antigens are typically used for these analysis (e.g. one specific for human nuclei (such as LMNA) and the second specific for human muscle fibers (typically Dystrophin)). Following immunofluorescent staining for the human antigens in the mouse muscle, the number of muscle fibers of human origin are counted and compared between treatments or samples.

### A. Identification

A cohort of immunodeficient mice (e.g., 21 NSG mice) is acclimated to facility (e.g., for at two days) prior to initiation of procedures. Mice are split into three groups (e.g. of 7) and identified with an ear notching procedure standard and/or routine to the facility.

### B. Cardiotoxin Tibialis Anterior Muscle Injury

A working solution of cardiotoxin (CTX) (e.g., 10 micromolar (μM)) is prepared by diluting the cardiotoxin stock solution in sterile buffer (e.g., Phosphate-Buffered Saline (PBS)) or in water before starting the procedure. For example, CTX used for this protocol can be a mixture of cardiotoxins having an average molecular weight of approximately 7,000 Da.

The mice are anesthetized by intraperitoneal injection of ketamine and xylazine (80 mg/kg and 10 mg/kg of body mass) or by Isoflurane inhalation (e.g. 3-4% in oxygen with maintenance as needed—typically 1-2%).

With the mouse face up, the hind limbs of the mice are sprayed with ethanol (e.g., 70% ethanol). Then, the hair at the anterior part of the lower leg (e.g., under the knee) is cut away (e.g., with the help of a scalpel) to visualize the exact location of the Tibialis Anterior (TA) muscle.

The CTX solution is drawn (e.g. about 100 microliter (μL)) into a microsyringe (e.g. 30 gauge Hamilton microsyringe) by pulling the plunger back slowly. Following, the CTX solution (e.g. 25 μL) can be injected into each TA muscle. For example, the needle of the syringe is inserted in the center of the TA by following the position of the tibia bone as guidance, from the tendon towards the knee. Since the TA is a thin tissue, the injection can be performed without going too deep (e.g. insert the needle ⅔ mm deep, at approximately a 10° to 20° angle), and avoid inserting the needle beyond the muscle itself.

After the procedure, the cage with the animal(s) is placed on a heated plate (e.g. 37° C.) until the mice are awake, since the anesthetic and the ethanol reduce body temperature.

### C. Myogenic Progenitor Cell Injection

After (e.g. 24 hours) CTX injection into both TA muscles, various groups of myogenic progenitor cells (e.g. 3×10e5 cells in 15 μl PBS) or blank buffer are injected into each mouse (e.g. left leg). The various groups can include isolated myogenic progenitor cells (e.g., as control) or myogenic progenitor cells prepared as provided herein. Each mouse receives one injection (e.g. in the left leg) of one of the cell preparations. Each mouse further receives one injection (e.g. in the right leg) of saline (e.g. 15 μl sterile PBS).

After injection, mice are returned to housing and monitored daily for humane endpoint conditions for a period of time (e.g. 4-8 weeks). The CTX injury model is typically repaired at a cellular level within a period of time (e.g. a month). At the end of the study (e.g. 4 weeks post-injection), mice are euthanized (e.g. by CO₂ asphyxiation with a secondary cervical dislocation) prior to tissue collection of the TA muscle for analyses (e.g. histological and molecular).

### D. Histological and Molecular Analysis

### Immunostaining

Cells (e.g., myogenetic progenitor cells as prepared herein prior to in vivo administration, cells isolated from the in vivo model) are fixed (e.g. with 4% PFA for 10 min at +4° C.) followed by permeabilization (e.g. with 0.1% Triton in PBS for 10 min at room temperature (RT)). After PBS wash, cells are blocked (e.g. for 30 min at RT with 3% BSA in PBS) and then incubated with primary antibodies diluted in blocking solution overnight at +4° C. Cells are then washed and incubated with secondary antibody diluted in blocking solution (e.g. supplemented with nuclear stain 4′,6-diamidino-2-phenylindole (DAPI) for 1 hour at RT). After PBS washes, cells are maintained in buffer (e.g. PBS) until final analysis. Pictures are acquired using an inverted fluorescence microscope.

Similarly, tissue cryosections (e.g., from the in vivo model) are permeabilized (e.g. with 0.3% Triton X-100 in PBS for 20 min at RT), then blocked and incubated with primary antibodies overnight. Sections are then washed and incubated with secondary antibodies.

### Quantification and Statistical Analysis

Histological analysis is performed (e.g. using the ImageJ distribution Fiji). Quantification of in vivo engraftment is performed by counting the number of muscle fibers derived from the myogenic progenitor cells as provided herein (e.g., human lamin A/C positive (hLMNA-C+) fibers for myogenic progenitor cells prepared from human cell-derived induced pluripotent stem cells) in a number of representative pictures (e.g. four) for each transplanted mouse (e.g. using a cell counter). The presence of hLMNA-C+fibers can indicate long-term homeostasis following stem cell therapy.

Alternatively or in addition to, analysis of myogenic differentiation potential of sorted subfractions is performed as follows: color channels are separated and threshold level for the red and blue channels are adjusted in order to select the area positive respectively to sample of interest (e.g. myosin heavy chain or MYHC (red)) and nuclear stain (e.g. DAPI (blue)). The area positive for each channel is analyzed (e.g. using Analyze Particle using 0-Infinity as Size parameter). Finally, the percentage of sample of interest (e.g. MYHC+) area for each image is normalized based on nuclear staining (e.g. DAPI+). Data represent mean±standard deviation of representative pictures (e.g. at least 2 pictures) for each independent experiments (e.g. n=3).

### Example 4: Differentiation of Myogenic Progenitor Cells

Similar to the experimental procedure provided in Example 1, a multi-step heterologous genetic circuit (FIG. 4A) can be designed up to promote stepwise progression of endogenous gene modulation in stem cell, to effect transformation of the stem cell to a differentiated cell (e.g., myocytes or myoblasts). Briefly, the stem cells (e.g. iPSCs) were transiently transfected with plasmid DNAs encoding one of the heterologous genetic circuits (“condition”) as described in FIG. 4B. Subsequently, cell differentiation analysis (e.g., staining, flow cytometry) revealed greater myoblast differentiation efficiency with heterologous genetic circuits (or conditions) 1 and 2.

TABLE 2
Example sequences of a proGuide encoding an activatable guide nucleic acid molecule
against a target gene.

| Target | SPACER | EXTRA | UPSTREAM | POLYT | DOWNSTREAM |
|---|---|---|---|---|---|
| Gene | SEQUENCE | SEQUENCE | STEM | UNIT | STEM |
| TBXT | SEQ ID NO: 1 | SEQ ID NO: | SEQ ID NO: | TTTTTT | SEQ ID NO: 38 |
| | GCTTTCACG | 32 | 35 | TTT | GCGCTCCTACGGA |
| | GAAGG | GTTTTAGA | CCTTCCCG | | AGTCGGGAAGG |
| | | GCTA | ACTTCCGT | | |
| | | | AGGAGCGC | | |
| TBXT | SEQ ID NO: 2 | SEQ ID NO: | SEQ ID NO: | TTTTTT | SEQ ID NO: 38 |
| | CTGCGCATC | 32 | 35 | TTT | |
| | GGGTG | | | | |
| TBXT | SEQ ID NO: 3 | SEQ ID NO: | SEQ ID NO: | TTTTTT | SEQ ID NO: 38 |
| | CCGCACTTC | 32 | 35 | TTT | |
| | CGCGA | | | | |
| TBXT | SEQ ID NO: 4 | SEQ ID NO: | SEQ ID NO: | TTTTTT | SEQ ID NO: 38 |
| | GCGCCAAGC | 32 | 35 | TTT | |
| | GGTGT | | | | |
| TBXT | SEQ ID NO: 1 | SEQ ID NO: | SEQ ID NO: | TTTTTT | SEQ ID NO: 39 |
| | GCTTTCACG | 32 | 36 | TTT | CGGCTTGGTATGCG |
| | GAAGG | | CCGGTGCT | | GAGCACCGG |
| | | | CCGCATAC | | |
| | | | CAAGCCG | | |
| TBXT | SEQ ID NO: 2 | SEQ ID NO: | SEQ ID NO: | TTTTTT | SEQ ID NO: 39 |
| | | 32 | 36 | TTT | |
| TBXT | SEQ ID NO: 3 | SEQ ID NO: | SEQ ID NO: | TTTTTT | SEQ ID NO: 39 |
| | | 32 | 36 | TTT | |
| TBXT | SEQ ID NO: 4 | SEQ ID NO: | SEQ ID NO: | TTTTTT | SEQ ID NO: 39 |
| | | 32 | 36 | TTT | |
| TBXT | SEQ ID NO: 1 | SEQ ID NO: | SEQ ID NO: | TTTTTT | SEQ ID NO: 40 |
| | | 32 | 37 | TTT | GAGGACCCGATCTT |
| | | | CCTGAAGG | | TACCTTCAGG |
| | | | TAAAGATC | | |
| | | | GGGTCCTC | | |
| TBXT | SEQ ID NO: 2 | SEQ ID NO: | SEQ ID NO: | TTTTTT | SEQ ID NO: 40 |
| | | 32 | 37 | TTT | |
| TBXT | SEQ ID NO: 3 | SEQ ID NO: | SEQ ID NO: | TTTTTT | SEQ ID NO: 40 |
| | | 32 | 37 | TTT | |
| TBXT | SEQ ID NO: 4 | SEQ ID NO: | SEQ ID NO: | TTTTTT | SEQ ID NO: 40 |
| | | 32 | 37 | TTT | |
| TBX6 | SEQ ID NO: 9 | SEQ ID NO: | SEQ ID NO: | TTTTTT | SEQ ID NO: 38 |
| | CGAGGTCAC | 32 | 35 | TTT | |
| | GGCGT | | | | |
| TBX6 | SEQ ID NO: 10 | SEQ ID NO: | SEQ ID NO: | TTTTTT | SEQ ID NO: 38 |
| | CCGCGTGGT | 32 | 35 | TTT | |
| | CCGTC | | | | |
| TBX6 | SEQ ID NO: 11 | SEQ ID NO: | SEQ ID NO: | TTTTTT | SEQ ID NO: 38 |
| | GGGGCAGCG | 32 | 35 | TTT | |
| | TCCCA | | | | |
| TBX6 | SEQ ID NO: 12 | SEQ ID NO: | SEQ ID NO: | TTTTTT | SEQ ID NO: 38 |
| | CCAGACCTT | 32 | 35 | TTT | |
| | CTGGC | | | | |
| TBX6 | SEQ ID NO: 9 | SEQ ID NO: | SEQ ID NO: | TTTTTT | SEQ ID NO: 39 |
| | | 32 | 36 | TTT | |
| TBX6 | SEQ ID NO: 10 | SEQ ID NO: | SEQ ID NO: | TTTTTT | SEQ ID NO: 39 |
| | | 32 | 36 | TTT | |
| TBX6 | SEQ ID NO: 11 | SEQ ID NO: | SEQ ID NO: | TTTTTT | SEQ ID NO: 39 |
| | | 32 | 36 | TTT | |
| TBX6 | SEQ ID NO: 12 | SEQ ID NO: | SEQ ID NO: | TTTTTT | SEQ ID NO: 39 |
| | | 32 | 36 | TTT | |
| TBX6 | SEQ ID NO: 9 | SEQ ID NO: | SEQ ID NO: | TTTTTT | SEQ ID NO: 40 |
| | | 32 | 37 | TTT | |
| TBX6 | SEQ ID NO: 10 | SEQ ID NO: | SEQ ID NO: | TTTTTT | SEQ ID NO: 40 |
| | | 32 | 37 | TTT | |
| TBX6 | SEQ ID NO: 11 | SEQ ID NO: | SEQ ID NO: | TTTTTT | SEQ ID NO: 40 |
| | | 32 | 37 | TTT | |
| TBX6 | SEQ ID NO: 12 | SEQ ID NO: | SEQ ID NO: | TTTTTT | SEQ ID NO: 40 |
| | | 32 | 37 | TTT | |
| MSGN1 | SEQ ID NO: 15 | SEQ ID NO: | SEQ ID NO: | TTTTTT | SEQ ID NO: 38 |
| | TATGCAGCC | 32 | 35 | TTT | |
| | TTAAT | | | | |
| MSGN1 | SEQ ID NO: 16 | SEQ ID NO: | SEQ ID NO: | TTTTTT | SEQ ID NO: 38 |
| | CTGCCGCCT | 32 | 35 | TTT | |
| | GGCCA | | | | |
| MSGN1 | SEQ ID NO: 17 | SEQ ID NO: | SEQ ID NO: | TTTTTT | SEQ ID NO: 38 |
| | AAAGTGACC | 32 | 35 | TTT | |
| | CGAGA | | | | |
| MSGN1 | SEQ ID NO: 18 | SEQ ID NO: | SEQ ID NO: | TTTTTT | SEQ ID NO: 38 |
| | GCTGCGGAT | 32 | 35 | TTT | |
| | GCCCC | | | | |
| MSGN1 | SEQ ID NO: 15 | SEQ ID NO: | SEQ ID NO: | TTTTTT | SEQ ID NO: 39 |
| | | 32 | 36 | TTT | |
| MSGN1 | SEQ ID NO: 16 | SEQ ID NO: | SEQ ID NO: | TTTTTT | SEQ ID NO: 39 |
| | | 32 | 36 | TTT | |
| MSGN1 | SEQ ID NO: 17 | SEQ ID NO: | SEQ ID NO: | TTTTTT | SEQ ID NO: 39 |
| | | 32 | 36 | TTT | |
| MSGN1 | SEQ ID NO: 18 | SEQ ID NO: | SEQ ID NO: | TTTTTT | SEQ ID NO: 39 |
| | | 32 | 36 | TTT | |
| MSGN1 | SEQ ID NO: 15 | SEQ ID NO: | SEQ ID NO: | TTTTTT | SEQ ID NO: 40 |
| | | 32 | 37 | TTT | |
| MSGN1 | SEQ ID NO: 16 | SEQ ID NO: | SEQ ID NO: | TTTTTT | SEQ ID NO: 40 |
| | | 32 | 37 | TTT | |
| MSGN1 | SEQ ID NO: 17 | SEQ ID NO: | SEQ ID NO: | TTTTTT | SEQ ID NO: 40 |
| | | 32 | 37 | TTT | |
| MSGN1 | SEQ ID NO: 18 | SEQ ID NO: | SEQ ID NO: | TTTTTT | SEQ ID NO: 40 |
| | | 32 | 37 | TTT | |
| PAX3 | SEQ ID NO: 22 | SEQ ID NO: | SEQ ID NO: | TTTTTT | SEQ ID NO: 38 |
| | GGGTAGACC | 32 | 35 | TTT | |
| | GTCCC | | | | |
| PAX3 | SEQ ID NO: 23 | SEQ ID NO: | SEQ ID NO: | TTTTTT | SEQ ID NO: 38 |
| | CGAGTGCGG | 32 | 35 | TTT | |
| | GGATC | | | | |
| PAX3 | SEQ ID NO: 24 | SEQ ID NO: | SEQ ID NO: | TTTTTT | SEQ ID NO: 38 |
| | GCACCACCA | 32 | 35 | TTT | |
| | GAGAT | | | | |
| PAX3 | SEQ ID NO: 25 | SEQ ID NO: | SEQ ID NO: | TTTTTT | SEQ ID NO: 38 |
| | CAACCTTCT | 32 | 35 | TTT | |
| | CTCCT | | | | |
| PAX3 | SEQ ID NO: 22 | SEQ ID NO: | SEQ ID NO: | TTTTTT | SEQ ID NO: 39 |
| | | 32 | 36 | TTT | |
| PAX3 | SEQ ID NO: 23 | SEQ ID NO: | SEQ ID NO: | TTTTTT | SEQ ID NO: 39 |
| | | 32 | 36 | TTT | |
| PAX3 | SEQ ID NO: 24 | SEQ ID NO: | SEQ ID NO: | TTTTTT | SEQ ID NO: 39 |
| | | 32 | 36 | TTT | |
| PAX3 | SEQ ID NO: 25 | SEQ ID NO: | SEQ ID NO: | TTTTTT | SEQ ID NO: 39 |
| | | 32 | 36 | TTT | |
| PAX3 | SEQ ID NO: 22 | SEQ ID NO: | SEQ ID NO: | TTTTTT | SEQ ID NO: 40 |
| | | 32 | 37 | TTT | |
| PAX3 | SEQ ID NO: 23 | SEQ ID NO: | SEQ ID NO: | TTTTTT | SEQ ID NO: 40 |
| | | 32 | 37 | TTT | |
| PAX3 | SEQ ID NO: 24 | SEQ ID NO: | SEQ ID NO: | TTTTTT | SEQ ID NO: 40 |
| | | 32 | 37 | TTT | |
| PAX3 | SEQ ID NO: 25 | SEQ ID NO: | SEQ ID NO: | TTTTTT | SEQ ID NO: 40 |
| | | 32 | 37 | TTT | |
| PAX7 | SEQ ID NO: 28 | SEQ ID NO: | SEQ ID NO: | TTTTTT | SEQ ID NO: 38 |
| | TAGGGTGAC | 32 | 35 | TTT | |
| | TTGAA | | | | |
| PAX7 | SEQ ID NO: 29 | SEQ ID NO: | SEQ ID NO: | TTTTTT | SEQ ID NO: 38 |
| | ACGAGGTCG | 32 | 35 | TTT | |
| | AGCCG | | | | |
| PAX7 | SEQ ID NO: 30 | SEQ ID NO: | SEQ ID NO: | TTTTTT | SEQ ID NO: 38 |
| | GGCCCATTC | 32 | 35 | TTT | |
| | ATCAA | | | | |
| PAX7 | SEQ ID NO: 31 | SEQ ID NO: | SEQ ID NO: | TTTTTT | SEQ ID NO: 38 |
| | GCTCCCTTG | 32 | 35 | TTT | |
| | CGCCC | | | | |
| PAX7 | SEQ ID NO: 28 | SEQ ID NO: | SEQ ID NO: | TTTTTT | SEQ ID NO: 39 |
| | | 32 | 36 | TTT | |
| PAX7 | SEQ ID NO: 29 | SEQ ID NO: | SEQ ID NO: | TTTTTT | SEQ ID NO: 39 |
| | | 32 | 36 | TTT | |
| PAX7 | SEQ ID NO: 30 | SEQ ID NO: | SEQ ID NO: | TTTTTT | SEQ ID NO: 39 |
| | | 32 | 36 | TTT | |
| PAX7 | SEQ ID NO: 31 | SEQ ID NO: | SEQ ID NO: | TTTTTT | SEQ ID NO: 39 |
| | | 32 | 36 | TTT | |
| PAX7 | SEQ ID NO: 28 | SEQ ID NO: | SEQ ID NO: | TTTTTT | SEQ ID NO: 40 |
| | | 32 | 37 | TTT | |
| PAX7 | SEQ ID NO: 29 | SEQ ID NO: | SEQ ID NO: | TTTTTT | SEQ ID NO: 40 |
| | | 32 | 37 | TTT | |
| PAX7 | SEQ ID NO: 30 | SEQ ID NO: | SEQ ID NO: | TTTTTT | SEQ ID NO: 40 |
| | | 32 | 37 | TTT | |
| PAX7 | SEQ ID NO: 31 | SEQ ID NO: | SEQ ID NO: | TTTTTT | SEQ ID NO: 40 |
| | | 32 | 37 | TTT | |

TABLE 3
Concatenated portion of the proGuide from Table 2

| | | Concatenated portion of a proGuide |
|---|---|---|
| Target | SEQ ID | (SPACER + EXTRA + UPSTREAM + POLYT_UNIT + |
| Gene | NO: | DOWNSTREAM STEM) |
| TBXT | 41 | GCTTTCACGGAAGGGTTTTAGAGCTACCTTCCCGACTTCCGTAGGAG |
| | | CGCTTTTTTTTTGCGCTCCTACGGAAGTCGGGAAGG |
| TBXT | 42 | CTGCGCATCGGGTGGTTTTAGAGCTACCTTCCCGACTTCCGTAGGAG |
| | | CGCTTTTTTTTTGCGCTCCTACGGAAGTCGGGAAGG |
| TBXT | 43 | CCGCACTTCCGCGAGTTTTAGAGCTACCTTCCCGACTTCCGTAGGAG |
| | | CGCTTTTTTTTTGCGCTCCTACGGAAGTCGGGAAGG |
| TBXT | 44 | GCGCCAAGCGGTGTGTTTTAGAGCTACCTTCCCGACTTCCGTAGGAG |
| | | CGCTTTTTTTTTGCGCTCCTACGGAAGTCGGGAAGG |
| TBXT | 45 | GCTTTCACGGAAGGGTTTTAGAGCTACCGGTGCTCCGCATACCAAGC |
| | | CGTTTTTTTTTCGGCTTGGTATGCGGAGCACCGG |
| TBXT | 46 | CTGCGCATCGGGTGGTTTTAGAGCTACCGGTGCTCCGCATACCAAGC |
| | | CGTTTTTTTTTCGGCTTGGTATGCGGAGCACCGG |
| TBXT | 47 | CCGCACTTCCGCGAGTTTTAGAGCTACCGGTGCTCCGCATACCAAGC |
| | | CGTTTTTTTTTCGGCTTGGTATGCGGAGCACCGG |
| TBXT | 48 | GCGCCAAGCGGTGTGTTTTAGAGCTACCGGTGCTCCGCATACCAAGC |
| | | CGTTTTTTTTTCGGCTTGGTATGCGGAGCACCGG |
| TBXT | 49 | GCTTTCACGGAAGGGTTTTAGAGCTACCTGAAGGTAAAGATCGGGT |
| | | CCTCTTTTTTTTTGAGGACCCGATCTTTACCTTCAGG |
| TBXT | 50 | CTGCGCATCGGGTGGTTTTAGAGCTACCTGAAGGTAAAGATCGGGT |
| | | CCTCTTTTTTTTTGAGGACCCGATCTTTACCTTCAGG |
| TBXT | 51 | CCGCACTTCCGCGAGTTTTAGAGCTACCTGAAGGTAAAGATCGGGTC |
| | | CTCTTTTTTTTTGAGGACCCGATCTTTACCTTCAGG |
| TBXT | 52 | GCGCCAAGCGGTGTGTTTTAGAGCTACCTGAAGGTAAAGATCGGGT |
| | | CCTCTTTTTTTTTGAGGACCCGATCTTTACCTTCAGG |
| TBX6 | 53 | CGAGGTCACGGCGTGTTTTAGAGCTACCTTCCCGACTTCCGTAGGAG |
| | | CGCTTTTTTTTTGCGCTCCTACGGAAGTCGGGAAGG |
| TBX6 | 54 | CCGCGTGGTCCGTCGTTTTAGAGCTACCTTCCCGACTTCCGTAGGAG |
| | | CGCTTTTTTTTTGCGCTCCTACGGAAGTCGGGAAGG |
| TBX6 | 55 | GGGGCAGCGTCCCAGTTTTAGAGCTACCTTCCCGACTTCCGTAGGAG |
| | | CGCTTTTTTTTTGCGCTCCTACGGAAGTCGGGAAGG |
| TBX6 | 56 | CCAGACCTTCTGGCGTTTTAGAGCTACCTTCCCGACTTCCGTAGGAG |
| | | CGCTTTTTTTTTGCGCTCCTACGGAAGTCGGGAAGG |
| TBX6 | 57 | CGAGGTCACGGCGTGTTTTAGAGCTACCGGTGCTCCGCATACCAAGC |
| | | CGTTTTTTTTTCGGCTTGGTATGCGGAGCACCGG |
| TBX6 | 58 | CCGCGTGGTCCGTCGTTTTAGAGCTACCGGTGCTCCGCATACCAAGC |
| | | CGTTTTTTTTTCGGCTTGGTATGCGGAGCACCGG |
| TBX6 | 59 | GGGGCAGCGTCCCAGTTTTAGAGCTACCGGTGCTCCGCATACCAAG |
| | | CCGTTTTTTTTTCGGCTTGGTATGCGGAGCACCGG |
| TBX6 | 60 | CCAGACCTTCTGGCGTTTTAGAGCTACCGGTGCTCCGCATACCAAGC |
| | | CGTTTTTTTTTCGGCTTGGTATGCGGAGCACCGG |
| TBX6 | 61 | CGAGGTCACGGCGTGTTTTAGAGCTACCTGAAGGTAAAGATCGGGT |
| | | CCTCTTTTTTTTTGAGGACCCGATCTTTACCTTCAGG |
| TBX6 | 62 | CCGCGTGGTCCGTCGTTTTAGAGCTACCTGAAGGTAAAGATCGGGTC |
| | | CTCTTTTTTTTTGAGGACCCGATCTTTACCTTCAGG |
| TBX6 | 63 | GGGGCAGCGTCCCAGTTTTAGAGCTACCTGAAGGTAAAGATCGGGT |
| | | CCTCTTTTTTTTTGAGGACCCGATCTTTACCTTCAGG |
| TBX6 | 64 | CCAGACCTTCTGGCGTTTTAGAGCTACCTGAAGGTAAAGATCGGGTC |
| | | CTCTTTTTTTTTGAGGACCCGATCTTTACCTTCAGG |
| MSGN1 | 65 | TATGCAGCCTTAATGTTTTAGAGCTACCTTCCCGACTTCCGTAGGAG |
| | | CGCTTTTTTTTTGCGCTCCTACGGAAGTCGGGAAGG |
| MSGN1 | 66 | CTGCCGCCTGGCCAGTTTTAGAGCTACCTTCCCGACTTCCGTAGGAG |
| | | CGCTTTTTTTTTGCGCTCCTACGGAAGTCGGGAAGG |
| MSGN1 | 67 | AAAGTGACCCGAGAGTTTTAGAGCTACCTTCCCGACTTCCGTAGGAG |
| | | CGCTTTTTTTTTGCGCTCCTACGGAAGTCGGGAAGG |
| MSGN1 | 68 | GCTGCGGATGCCCCGTTTTAGAGCTACCTTCCCGACTTCCGTAGGAG |
| | | CGCTTTTTTTTTGCGCTCCTACGGAAGTCGGGAAGG |
| MSGN1 | 69 | TATGCAGCCTTAATGTTTTAGAGCTACCGGTGCTCCGCATACCAAGC |
| | | CGTTTTTTTTTCGGCTTGGTATGCGGAGCACCGG |
| MSGN1 | 70 | CTGCCGCCTGGCCAGTTTTAGAGCTACCGGTGCTCCGCATACCAAGC |
| | | CGTTTTTTTTTCGGCTTGGTATGCGGAGCACCGG |
| MSGN1 | 71 | AAAGTGACCCGAGAGTTTTAGAGCTACCGGTGCTCCGCATACCAAG |
| | | CCGTTTTTTTTTCGGCTTGGTATGCGGAGCACCGG |
| MSGN1 | 72 | GCTGCGGATGCCCCGTTTTAGAGCTACCGGTGCTCCGCATACCAAGC |
| | | CGTTTTTTTTTCGGCTTGGTATGCGGAGCACCGG |
| MSGN1 | 73 | TATGCAGCCTTAATGTTTTAGAGCTACCTGAAGGTAAAGATCGGGTC |
| | | CTCTTTTTTTTTGAGGACCCGATCTTTACCTTCAGG |
| MSGN1 | 74 | CTGCCGCCTGGCCAGTTTTAGAGCTACCTGAAGGTAAAGATCGGGTC |
| | | CTCTTTTTTTTTGAGGACCCGATCTTTACCTTCAGG |
| MSGN1 | 75 | AAAGTGACCCGAGAGTTTTAGAGCTACCTGAAGGTAAAGATCGGGT |
| | | CCTCTTTTTTTTTGAGGACCCGATCTTTACCTTCAGG |
| MSGN1 | 76 | GCTGCGGATGCCCCGTTTTAGAGCTACCTGAAGGTAAAGATCGGGT |
| | | CCTCTTTTTTTTTGAGGACCCGATCTTTACCTTCAGG |
| PAX3 | 77 | GGGTAGACCGTCCCGTTTTAGAGCTACCTTCCCGACTTCCGTAGGAG |
| | | CGCTTTTTTTTTGCGCTCCTACGGAAGTCGGGAAGG |
| PAX3 | 78 | CGAGTGCGGGGATCGTTTTAGAGCTACCTTCCCGACTTCCGTAGGAG |
| | | CGCTTTTTTTTTGCGCTCCTACGGAAGTCGGGAAGG |
| PAX3 | 79 | GCACCACCAGAGATGTTTTAGAGCTACCTTCCCGACTTCCGTAGGAG |
| | | CGCTTTTTTTTTGCGCTCCTACGGAAGTCGGGAAGG |
| PAX3 | 80 | CAACCTTCTCTCCTGTTTTAGAGCTACCTTCCCGACTTCCGTAGGAGC |
| | | GCTTTTTTTTTGCGCTCCTACGGAAGTCGGGAAGG |
| PAX3 | 81 | GGGTAGACCGTCCCGTTTTAGAGCTACCGGTGCTCCGCATACCAAGC |
| | | CGTTTTTTTTTCGGCTTGGTATGCGGAGCACCGG |
| PAX3 | 82 | CGAGTGCGGGGATCGTTTTAGAGCTACCGGTGCTCCGCATACCAAG |
| | | CCGTTTTTTTTTCGGCTTGGTATGCGGAGCACCGG |
| PAX3 | 83 | GCACCACCAGAGATGTTTTAGAGCTACCGGTGCTCCGCATACCAAG |
| | | CCGTTTTTTTTTCGGCTTGGTATGCGGAGCACCGG |
| PAX3 | 84 | CAACCTTCTCTCCTGTTTTAGAGCTACCGGTGCTCCGCATACCAAGC |
| | | CGTTTTTTTTTCGGCTTGGTATGCGGAGCACCGG |
| PAX3 | 85 | GGGTAGACCGTCCCGTTTTAGAGCTACCTGAAGGTAAAGATCGGGT |
| | | CCTCTTTTTTTTTGAGGACCCGATCTTTACCTTCAGG |
| PAX3 | 86 | CGAGTGCGGGGATCGTTTTAGAGCTACCTGAAGGTAAAGATCGGGT |
| | | CCTCTTTTTTTTTGAGGACCCGATCTTTACCTTCAGG |
| PAX3 | 87 | GCACCACCAGAGATGTTTTAGAGCTACCTGAAGGTAAAGATCGGGT |
| | | CCTCTTTTTTTTTGAGGACCCGATCTTTACCTTCAGG |
| PAX3 | 88 | CAACCTTCTCTCCTGTTTTAGAGCTACCTGAAGGTAAAGATCGGGTC |
| | | CTCTTTTTTTTTGAGGACCCGATCTTTACCTTCAGG |
| PAX7 | 89 | TAGGGTGACTTGAAGTTTTAGAGCTACCTTCCCGACTTCCGTAGGAG |
| | | CGCTTTTTTTTTGCGCTCCTACGGAAGTCGGGAAGG |
| PAX7 | 90 | ACGAGGTCGAGCCGGTTTTAGAGCTACCTTCCCGACTTCCGTAGGAG |
| | | CGCTTTTTTTTTGCGCTCCTACGGAAGTCGGGAAGG |
| PAX7 | 91 | GGCCCATTCATCAAGTTTTAGAGCTACCTTCCCGACTTCCGTAGGAG |
| | | CGCTTTTTTTTTGCGCTCCTACGGAAGTCGGGAAGG |
| PAX7 | 92 | GCTCCCTTGCGCCCGTTTTAGAGCTACCTTCCCGACTTCCGTAGGAG |
| | | CGCTTTTTTTTTGCGCTCCTACGGAAGTCGGGAAGG |
| PAX7 | 93 | TAGGGTGACTTGAAGTTTTAGAGCTACCGGTGCTCCGCATACCAAGC |
| | | CGTTTTTTTTTCGGCTTGGTATGCGGAGCACCGG |
| PAX7 | 94 | ACGAGGTCGAGCCGGTTTTAGAGCTACCGGTGCTCCGCATACCAAG |
| | | CCGTTTTTTTTTCGGCTTGGTATGCGGAGCACCGG |
| PAX7 | 95 | GGCCCATTCATCAAGTTTTAGAGCTACCGGTGCTCCGCATACCAAGC |
| | | CGTTTTTTTTTCGGCTTGGTATGCGGAGCACCGG |
| PAX7 | 96 | GCTCCCTTGCGCCCGTTTTAGAGCTACCGGTGCTCCGCATACCAAGC |
| | | CGTTTTTTTTTCGGCTTGGTATGCGGAGCACCGG |
| PAX7 | 97 | TAGGGTGACTTGAAGTTTTAGAGCTACCTGAAGGTAAAGATCGGGT |
| | | CCTCTTTTTTTTTGAGGACCCGATCTTTACCTTCAGG |
| PAX7 | 98 | ACGAGGTCGAGCCGGTTTTAGAGCTACCTGAAGGTAAAGATCGGGT |
| | | CCTCTTTTTTTTTGAGGACCCGATCTTTACCTTCAGG |
| PAX7 | 99 | GGCCCATTCATCAAGTTTTAGAGCTACCTGAAGGTAAAGATCGGGTC |
| | | CTCTTTTTTTTTGAGGACCCGATCTTTACCTTCAGG |
| PAX7 | 100 | GCTCCCTTGCGCCCGTTTTAGAGCTACCTGAAGGTAAAGATCGGGTC |
| | | CTCTTTTTTTTTGAGGACCCGATCTTTACCTTCAGG |

TABLE 4
Example sequences of a proGuide encoding an activatable guide nucleic acid molecule
against a target gene.

| Target | SPACER | EXTRA | UPSTREAM | POLYT | DOWNSTREAM |
|---|---|---|---|---|---|
| gene | SEQUENCE | SEQUENCE | STEM | UNIT | STEM |
| TBXT | SEQ ID NO: 1 | SEQ ID NO: | SEQ ID NO: 35 | SEQ ID | SEQ ID NO: 38 |
| | GCTTTCACG | 32 | CCTTCCCGACT | NO: 34 | GCGCTCCTACG |
| | GAAGG | GTTTTAGA | TCCGTAGGAG | TTTTT | GAAGTCGGGA |
| | | GCTA | CGC | TTTT | AGG |
| TBXT | SEQ ID NO: 2 | SEQ ID NO: | SEQ ID NO: 35 | SEQ ID | SEQ ID NO: 38 |
| | CTGCGCATC | 32 | | NO: 34 | |
| | GGGTG | | | | |
| TBXT | SEQ ID NO: 3 | SEQ ID NO: | SEQ ID NO: 35 | SEQ ID | SEQ ID NO: 38 |
| | CCGCACTTC | 32 | | NO: 34 | |
| | CGCGA | | | | |
| TBXT | SEQ ID NO: 4 | SEQ ID NO: | SEQ ID NO: 35 | SEQ ID | SEQ ID NO: 38 |
| | GCGCCAAGC | 32 | | NO: 34 | |
| | GGTGT | | | | |
| TBXT | SEQ ID NO: 1 | SEQ ID NO: | SEQ ID NO: 36 | SEQ ID | SEQ ID NO: 39 |
| | | 32 | CCGGTGCTCC | NO: 34 | CGGCTTGGTAT |
| | | | GCATACCAAG | | GCGGAGCACCG |
| | | | CCG | | G |
| TBXT | SEQ ID NO: 2 | SEQ ID NO: | SEQ ID NO: 36 | SEQ ID | SEQ ID NO: 39 |
| | | 32 | | NO: 34 | |
| TBXT | SEQ ID NO: 3 | SEQ ID NO: | SEQ ID NO: 36 | SEQ ID | SEQ ID NO: 39 |
| | | 32 | | NO: 34 | |
| TBXT | SEQ ID NO: 4 | SEQ ID NO: | SEQ ID NO: 36 | SEQ ID | SEQ ID NO: 39 |
| | | 32 | | NO: 34 | |
| TBXT | SEQ ID NO: 1 | SEQ ID NO: | SEQ ID NO: 37 | SEQ ID | SEQ ID NO: 40 |
| | | 32 | CCTGAAGGTA | NO: 34 | GAGGACCCGAT |
| | | | AAGATCGGGT | | CTTTACCTTCA |
| | | | CCTC | | GG |
| TBXT | SEQ ID NO: 2 | SEQ ID NO: | SEQ ID NO: 37 | SEQ ID | SEQ ID NO: 40 |
| | | 32 | | NO: 34 | |
| TBXT | SEQ ID NO: 3 | SEQ ID NO: | SEQ ID NO: 37 | SEQ ID | SEQ ID NO: 40 |
| | | 32 | | NO: 34 | |
| TBXT | SEQ ID NO: 4 | SEQ ID NO: | SEQ ID NO: 37 | SEQ ID | SEQ ID NO: 40 |
| | | 32 | | NO: 34 | |
| TBX6 | SEQ ID NO: 9 | SEQ ID NO: | SEQ ID NO: 35 | SEQ ID | SEQ ID NO: 38 |
| | CGAGGTCAC | 32 | | NO: 34 | |
| | GGCGT | | | | |
| TBX6 | SEQ ID NO: 10 | SEQ ID NO: | SEQ ID NO: 35 | SEQ ID | SEQ ID NO: 38 |
| | CCGCGTGGT | 32 | | NO: 34 | |
| | CCGTC | | | | |
| TBX6 | SEQ ID NO: 11 | SEQ ID NO: | SEQ ID NO: 35 | SEQ ID | SEQ ID NO: 38 |
| | GGGGCAGCG | 32 | | NO: 34 | |
| | TCCCA | | | | |
| TBX6 | SEQ ID NO: 12 | SEQ ID NO: | SEQ ID NO: 35 | SEQ ID | SEQ ID NO: 38 |
| | CCAGACCTT | 32 | | NO: 34 | |
| | CTGGC | | | | |
| TBX6 | SEQ ID NO: 9 | SEQ ID NO: | SEQ ID NO: 36 | SEQ ID | SEQ ID NO: 39 |
| | | 32 | | NO: 34 | |
| TBX6 | SEQ ID NO: 10 | SEQ ID NO: | SEQ ID NO: 36 | SEQ ID | SEQ ID NO: 39 |
| | | 32 | | NO: 34 | |
| TBX6 | SEQ ID NO: 11 | SEQ ID NO: | SEQ ID NO: 36 | SEQ ID | SEQ ID NO: 39 |
| | | 32 | | NO: 34 | |
| TBX6 | SEQ ID NO: 12 | SEQ ID NO: | SEQ ID NO: 36 | SEQ ID | SEQ ID NO: 39 |
| | | 32 | | NO: 34 | |
| TBX6 | SEQ ID NO: 9 | SEQ ID NO: | SEQ ID NO: 37 | SEQ ID | SEQ ID NO: 40 |
| | | 32 | | NO: 34 | |
| TBX6 | SEQ ID NO: 10 | SEQ ID NO: | SEQ ID NO: 37 | SEQ ID | SEQ ID NO: 40 |
| | | 32 | | NO: 34 | |
| TBX6 | SEQ ID NO: 11 | SEQ ID NO: | SEQ ID NO: 37 | SEQ ID | SEQ ID NO: 40 |
| | | 32 | | NO: 34 | |
| TBX6 | SEQ ID NO: 12 | SEQ ID NO: | SEQ ID NO: 37 | SEQ ID | SEQ ID NO: 40 |
| | | 32 | | NO: 34 | |
| MSGN1 | SEQ ID NO: 15 | SEQ ID NO: | SEQ ID NO: 35 | SEQ ID | SEQ ID NO: 38 |
| | TATGCAGCC | 32 | | NO: 34 | |
| | TTAAT | | | | |
| MSGN1 | SEQ ID NO: 16 | SEQ ID NO: | SEQ ID NO: 35 | SEQ ID | SEQ ID NO: 38 |
| | CTGCCGCCT | 32 | | NO: 34 | |
| | GGCCA | | | | |
| MSGN1 | SEQ ID NO: 17 | SEQ ID NO: | SEQ ID NO: 35 | SEQ ID | SEQ ID NO: 38 |
| | AAAGTGACC | 32 | | NO: 34 | |
| | CGAGA | | | | |
| MSGN1 | SEQ ID NO: 18 | SEQ ID NO: | SEQ ID NO: 35 | SEQ ID | SEQ ID NO: 38 |
| | GCTGCGGAT | 32 | | NO: 34 | |
| | GCCCC | | | | |
| MSGN1 | SEQ ID NO: 15 | SEQ ID NO: | SEQ ID NO: 36 | SEQ ID | SEQ ID NO: 39 |
| | | 32 | | NO: 34 | |
| MSGN1 | SEQ ID NO: 16 | SEQ ID NO: | SEQ ID NO: 36 | SEQ ID | SEQ ID NO: 39 |
| | | 32 | | NO: 34 | |
| MSGN1 | SEQ ID NO: 17 | SEQ ID NO: | SEQ ID NO: 36 | SEQ ID | SEQ ID NO: 39 |
| | | 32 | | NO: 34 | |
| MSGN1 | SEQ ID NO: 18 | SEQ ID NO: | SEQ ID NO: 36 | SEQ ID | SEQ ID NO: 39 |
| | | 32 | | NO: 34 | |
| MSGN1 | SEQ ID NO: 15 | SEQ ID NO: | SEQ ID NO: 37 | SEQ ID | SEQ ID NO: 40 |
| | | 32 | | NO: 34 | |
| MSGN1 | SEQ ID NO: 16 | SEQ ID NO: | SEQ ID NO: 37 | SEQ ID | SEQ ID NO: 40 |
| | | 32 | | NO: 34 | |
| MSGN1 | SEQ ID NO: 17 | SEQ ID NO: | SEQ ID NO: 37 | SEQ ID | SEQ ID NO: 40 |
| | | 32 | | NO: 34 | |
| MSGN1 | SEQ ID NO: 18 | SEQ ID NO: | SEQ ID NO: 37 | SEQ ID | SEQ ID NO: 40 |
| | | 32 | | NO: 34 | |
| PAX3 | SEQ ID NO: 22 | SEQ ID NO: | SEQ ID NO: 35 | SEQ ID | SEQ ID NO: 38 |
| | GGGTAGACC | 32 | | NO: 34 | |
| | GTCCC | | | | |
| PAX3 | SEQ ID NO: 23 | SEQ ID NO: | SEQ ID NO: 35 | SEQ ID | SEQ ID NO: 38 |
| | CGAGTGCGG | 32 | | NO: 34 | |
| | GGATC | | | | |
| PAX3 | SEQ ID NO: 24 | SEQ ID NO: | SEQ ID NO: 35 | SEQ ID | SEQ ID NO: 38 |
| | GCACCACCA | 32 | | NO: 34 | |
| | GAGAT | | | | |
| PAX3 | SEQ ID NO: 25 | SEQ ID NO: | SEQ ID NO: 35 | SEQ ID | SEQ ID NO: 38 |
| | CAACCTTCT | 32 | | NO: 34 | |
| | CTCCT | | | | |
| PAX3 | SEQ ID NO: 22 | SEQ ID NO: | SEQ ID NO: 36 | SEQ ID | SEQ ID NO: 39 |
| | | 32 | | NO: 34 | |
| PAX3 | SEQ ID NO: 23 | SEQ ID NO: | SEQ ID NO: 36 | SEQ ID | SEQ ID NO: 39 |
| | | 32 | | NO: 34 | |
| PAX3 | SEQ ID NO: 24 | SEQ ID NO: | SEQ ID NO: 36 | SEQ ID | SEQ ID NO: 39 |
| | | 32 | | NO: 34 | |
| PAX3 | SEQ ID NO: 25 | SEQ ID NO: | SEQ ID NO: 36 | SEQ ID | SEQ ID NO: 39 |
| | | 32 | | NO: 34 | |
| PAX3 | SEQ ID NO: 22 | SEQ ID NO: | SEQ ID NO: 37 | SEQ ID | SEQ ID NO: 40 |
| | | 32 | | NO: 34 | |
| PAX3 | SEQ ID NO: 23 | SEQ ID NO: | SEQ ID NO: 37 | SEQ ID | SEQ ID NO: 40 |
| | | 32 | | NO: 34 | |
| PAX3 | SEQ ID NO: 24 | SEQ ID NO: | SEQ ID NO: 37 | SEQ ID | SEQ ID NO: 40 |
| | | 32 | | NO: 34 | |
| PAX3 | SEQ ID NO: 25 | SEQ ID NO: | SEQ ID NO: 37 | SEQ ID | SEQ ID NO: 40 |
| | | 32 | | NO: 34 | |
| PAX7 | SEQ ID NO: 28 | SEQ ID NO: | SEQ ID NO: 35 | SEQ ID | SEQ ID NO: 38 |
| | TAGGGTGAC | 32 | | NO: 34 | |
| | TTGAA | | | | |
| PAX7 | SEQ ID NO: 29 | SEQ ID NO: | SEQ ID NO: 35 | SEQ ID | SEQ ID NO: 38 |
| | ACGAGGTCG | 32 | | NO: 34 | |
| | AGCCG | | | | |
| PAX7 | SEQ ID NO: 30 | SEQ ID NO: | SEQ ID NO: 35 | SEQ ID | SEQ ID NO: 38 |
| | GGCCCATTC | 32 | | NO: 34 | |
| | ATCAA | | | | |
| PAX7 | SEQ ID NO: 31 | SEQ ID NO: | SEQ ID NO: 35 | SEQ ID | SEQ ID NO: 38 |
| | GCTCCCTTG | 32 | | NO: 34 | |
| | CGCCC | | | | |
| PAX7 | SEQ ID NO: 28 | SEQ ID NO: | SEQ ID NO: 36 | SEQ ID | SEQ ID NO: 39 |
| | | 32 | | NO: 34 | |
| PAX7 | SEQ ID NO: 29 | SEQ ID NO: | SEQ ID NO: 36 | SEQ ID | SEQ ID NO: 39 |
| | | 32 | | NO: 34 | |
| PAX7 | SEQ ID NO: 30 | SEQ ID NO: | SEQ ID NO: 36 | SEQ ID | SEQ ID NO: 39 |
| | | 32 | | NO: 34 | |
| PAX7 | SEQ ID NO: 31 | SEQ ID NO: | SEQ ID NO: 36 | SEQ ID | SEQ ID NO: 39 |
| | | 32 | | NO: 34 | |
| PAX7 | SEQ ID NO: 28 | SEQ ID NO: | SEQ ID NO: 37 | SEQ ID | SEQ ID NO: 40 |
| | | 32 | | NO: 34 | |
| PAX7 | SEQ ID NO: 29 | SEQ ID NO: | SEQ ID NO: 37 | SEQ ID | SEQ ID NO: 40 |
| | | 32 | | NO: 34 | |
| PAX7 | SEQ ID NO: 30 | SEQ ID NO: | SEQ ID NO: 37 | SEQ ID | SEQ ID NO: 40 |
| | | 32 | | NO: 34 | |
| PAX7 | SEQ ID NO: 31 | SEQ ID NO: | SEQ ID NO: 37 | SEQ ID | SEQ ID NO: 40 |
| | | 32 | | NO: 34 | |
| TBXT | SEQ ID NO: 1 | SEQ ID NO: | SEQ ID NO: 431 | SEQ ID | SEQ ID NO: 440 |
| | | 32 | TAGCTACCGA | NO: 34 | CCTACACTCGA |
| | | | TGTCGAGTGT | | CATCGGTAGCT |
| | | | AGG | | A |
| TBXT | SEQ ID NO: 1 | SEQ ID NO: | SEQ ID NO: 432 | SEQ ID | SEQ ID NO: 441 |
| | | 32 | ATTACTCGAA | NO: 34 | CCCTGGCGGAA |
| | | | CGTTCCGCCA | | CGTTCGAGTAA |
| | | | GGG | | T |
| TBXT | SEQ ID NO: 1 | SEQ ID NO: | SEQ ID NO: 433 | SEQ ID | SEQ ID NO: 442 |
| | | 32 | GCGCACGACC | NO: 34 | CCTACACGATA |
| | | | ACTATCGTGT | | GTGGTCGTGCG |
| | | | AGG | | C |
| TBXT | SEQ ID NO: 1 | SEQ ID NO: | SEQ ID NO: 434 | SEQ ID | SEQ ID NO: 443 |
| | | 32 | ACTCGTTCGAT | NO: 34 | CCCGAACTCTC |
| | | | AGAGAGTTCG | | TATCGAACGAG |
| | | | GG | | T |
| TBXT | SEQ ID NO: 1 | SEQ ID NO: | SEQ ID NO: 435 | SEQ ID | SEQ ID NO: 444 |
| | | 32 | CCTCCGTGTCG | NO: 34 | CCATGGCACGA |
| | | | ATCGTGCCAT | | TCGACACGGAG |
| | | | GG | | G |
| TBXT | SEQ ID NO: 1 | SEQ ID NO: | SEQ ID NO: 436 | SEQ ID | SEQ ID NO: 445 |
| | | 32 | GCTCAGTCGC | NO: 34 | CCAAAGCTCAT |
| | | | GAATGAGCTT | | TCGCGACTGAG |
| | | | TGG | | C |
| TBXT | SEQ ID NO: 1 | SEQ ID NO: | SEQ ID NO: 437 | SEQ ID | SEQ ID NO: 446 |
| | | 32 | TAGCTCCCGTC | NO: 34 | CCGACGTCTAC |
| | | | CGTAGACGTC | | GGACGGGAGCT |
| | | | GG | | A |
| TBXT | SEQ ID NO: 1 | SEQ ID NO: | SEQ ID NO: 438 | SEQ ID | SEQ ID NO: 447 |
| | | 32 | TGCGTCGTCTA | NO: 34 | CCCGAGAGGTA |
| | | | CTACCTCTCGG | | GTAGACGACGC |
| | | | G | | A |
| TBXT | SEQ ID NO: 1 | SEQ ID NO: | SEQ ID NO: 439 | SEQ ID | SEQ ID NO: 448 |
| | | 32 | TGCTACGCAT | NO: 34 | CCCGTCGTCAC |
| | | | ACGTGACGAC | | GTATGCGTAGC |
| | | | GGG | | A |
| TBXT | SEQ ID NO: 2 | SEQ ID NO: | SEQ ID NO: 431 | SEQ ID | SEQ ID NO: 440 |
| | | 32 | | NO: 34 | |
| TBXT | SEQ ID NO: 2 | SEQ ID NO: | SEQ ID NO: 432 | SEQ ID | SEQ ID NO: 441 |
| | | 32 | | NO: 34 | |
| TBXT | SEQ ID NO: 2 | SEQ ID NO: | SEQ ID NO: 433 | SEQ ID | SEQ ID NO: 442 |
| | | 32 | | NO: 34 | |
| TBXT | SEQ ID NO: 2 | SEQ ID NO: | SEQ ID NO: 434 | SEQ ID | SEQ ID NO: 443 |
| | | 32 | | NO: 34 | |
| TBXT | SEQ ID NO: 2 | SEQ ID NO: | SEQ ID NO: 435 | SEQ ID | SEQ ID NO: 444 |
| | | 32 | | NO: 34 | |
| TBXT | SEQ ID NO: 2 | SEQ ID NO: | SEQ ID NO: 436 | SEQ ID | SEQ ID NO: 445 |
| | | 32 | | NO: 34 | |
| TBXT | SEQ ID NO: 2 | SEQ ID NO: | SEQ ID NO: 437 | SEQ ID | SEQ ID NO: 446 |
| | | 32 | | NO: 34 | |
| TBXT | SEQ ID NO: 2 | SEQ ID NO: | SEQ ID NO: 438 | SEQ ID | SEQ ID NO: 447 |
| | | 32 | | NO: 34 | |
| TBXT | SEQ ID NO: 2 | SEQ ID NO: | SEQ ID NO: 439 | SEQ ID | SEQ ID NO: 448 |
| | | 32 | | NO: 34 | |
| TBXT | SEQ ID NO: 3 | SEQ ID NO: | SEQ ID NO: 431 | SEQ ID | SEQ ID NO: 440 |
| | | 32 | | NO: 34 | |
| TBXT | SEQ ID NO: 3 | SEQ ID NO: | SEQ ID NO: 432 | SEQ ID | SEQ ID NO: 441 |
| | | 32 | | NO: 34 | |
| TBXT | SEQ ID NO: 3 | SEQ ID NO: | SEQ ID NO: 433 | SEQ ID | SEQ ID NO: 442 |
| | | 32 | | NO: 34 | |
| TBXT | SEQ ID NO: 3 | SEQ ID NO: | SEQ ID NO: 434 | SEQ ID | SEQ ID NO: 443 |
| | | 32 | | NO: 34 | |
| TBXT | SEQ ID NO: 3 | SEQ ID NO: | SEQ ID NO: 435 | SEQ ID | SEQ ID NO: 444 |
| | | 32 | | NO: 34 | |
| TBXT | SEQ ID NO: 3 | SEQ ID NO: | SEQ ID NO: 436 | SEQ ID | SEQ ID NO: 445 |
| | | 32 | | NO: 34 | |
| TBXT | SEQ ID NO: 3 | SEQ ID NO: | SEQ ID NO: 437 | SEQ ID | SEQ ID NO: 446 |
| | | 32 | | NO: 34 | |
| TBXT | SEQ ID NO: 3 | SEQ ID NO: | SEQ ID NO: 438 | SEQ ID | SEQ ID NO: 447 |
| | | 32 | | NO: 34 | |
| TBXT | SEQ ID NO: 3 | SEQ ID NO: | SEQ ID NO: 439 | SEQ ID | SEQ ID NO: 448 |
| | | 32 | | NO: 34 | |
| TBXT | SEQ ID NO: 4 | SEQ ID NO: | SEQ ID NO: 431 | SEQ ID | SEQ ID NO: 440 |
| | | 32 | | NO: 34 | |
| TBXT | SEQ ID NO: 4 | SEQ ID NO: | SEQ ID NO: 432 | SEQ ID | SEQ ID NO: 441 |
| | | 32 | | NO: 34 | |
| TBXT | SEQ ID NO: 4 | SEQ ID NO: | SEQ ID NO: 433 | SEQ ID | SEQ ID NO: 442 |
| | | 32 | | NO: 34 | |
| TBXT | SEQ ID NO: 4 | SEQ ID NO: | SEQ ID NO: 434 | SEQ ID | SEQ ID NO: 443 |
| | | 32 | | NO: 34 | |
| TBXT | SEQ ID NO: 4 | SEQ ID NO: | SEQ ID NO: 435 | SEQ ID | SEQ ID NO: 444 |
| | | 32 | | NO: 34 | |
| TBXT | SEQ ID NO: 4 | SEQ ID NO: | SEQ ID NO: 436 | SEQ ID | SEQ ID NO: 445 |
| | | 32 | | NO: 34 | |
| TBXT | SEQ ID NO: 4 | SEQ ID NO: | SEQ ID NO: 437 | SEQ ID | SEQ ID NO: 446 |
| | | 32 | | NO: 34 | |
| TBXT | SEQ ID NO: 4 | SEQ ID NO: | SEQ ID NO: 438 | SEQ ID | SEQ ID NO: 447 |
| | | 32 | | NO: 34 | |
| TBXT | SEQ ID NO: 4 | SEQ ID NO: | SEQ ID NO: 439 | SEQ ID | SEQ ID NO: 448 |
| | | 32 | | NO: 34 | |
| TBX6 | SEQ ID NO: 9 | SEQ ID NO: | SEQ ID NO: 431 | SEQ ID | SEQ ID NO: 440 |
| | | 32 | | NO: 34 | |
| TBX6 | SEQ ID NO: 9 | SEQ ID NO: | SEQ ID NO: 432 | SEQ ID | SEQ ID NO: 441 |
| | | 32 | | NO: 34 | |
| TBX6 | SEQ ID NO: 9 | SEQ ID NO: | SEQ ID NO: 433 | SEQ ID | SEQ ID NO: 442 |
| | | 32 | | NO: 34 | |
| TBX6 | SEQ ID NO: 9 | SEQ ID NO: | SEQ ID NO: 434 | SEQ ID | SEQ ID NO: 443 |
| | | 32 | | NO: 34 | |
| TBX6 | SEQ ID NO: 9 | SEQ ID NO: | SEQ ID NO: 435 | SEQ ID | SEQ ID NO: 444 |
| | | 32 | | NO: 34 | |
| TBX6 | SEQ ID NO: 9 | SEQ ID NO: | SEQ ID NO: 436 | SEQ ID | SEQ ID NO: 445 |
| | | 32 | | NO: 34 | |
| TBX6 | SEQ ID NO: 9 | SEQ ID NO: | SEQ ID NO: 437 | SEQ ID | SEQ ID NO: 446 |
| | | 32 | | NO: 34 | |
| TBX6 | SEQ ID NO: 9 | SEQ ID NO: | SEQ ID NO: 438 | SEQ ID | SEQ ID NO: 447 |
| | | 32 | | NO: 34 | |
| TBX6 | SEQ ID NO: 9 | SEQ ID NO: | SEQ ID NO: 439 | SEQ ID | SEQ ID NO: 448 |
| | | 32 | | NO: 34 | |
| TBX6 | SEQ ID NO: 10 | SEQ ID NO: | SEQ ID NO: 431 | SEQ ID | SEQ ID NO: 440 |
| | | 32 | | NO: 34 | |
| TBX6 | SEQ ID NO: 10 | SEQ ID NO: | SEQ ID NO: 432 | SEQ ID | SEQ ID NO: 441 |
| | | 32 | | NO: 34 | |
| TBX6 | SEQ ID NO: 10 | SEQ ID NO: | SEQ ID NO: 433 | SEQ ID | SEQ ID NO: 442 |
| | | 32 | | NO: 34 | |
| TBX6 | SEQ ID NO: 10 | SEQ ID NO: | SEQ ID NO: 434 | SEQ ID | SEQ ID NO: 443 |
| | | 32 | | NO: 34 | |
| TBX6 | SEQ ID NO: 10 | SEQ ID NO: | SEQ ID NO: 435 | SEQ ID | SEQ ID NO: 444 |
| | | 32 | | NO: 34 | |
| TBX6 | SEQ ID NO: 10 | SEQ ID NO: | SEQ ID NO: 436 | SEQ ID | SEQ ID NO: 445 |
| | | 32 | | NO: 34 | |
| TBX6 | SEQ ID NO: 10 | SEQ ID NO: | SEQ ID NO: 437 | SEQ ID | SEQ ID NO: 446 |
| | | 32 | | NO: 34 | |
| TBX6 | SEQ ID NO: 10 | SEQ ID NO: | SEQ ID NO: 438 | SEQ ID | SEQ ID NO: 447 |
| | | 32 | | NO: 34 | |
| TBX6 | SEQ ID NO: 10 | SEQ ID NO: | SEQ ID NO: 439 | SEQ ID | SEQ ID NO: 448 |
| | | 32 | | NO: 34 | |
| TBX6 | SEQ ID NO: 11 | SEQ ID NO: | SEQ ID NO: 431 | SEQ ID | SEQ ID NO: 440 |
| | | 32 | | NO: 34 | |
| TBX6 | SEQ ID NO: 11 | SEQ ID NO: | SEQ ID NO: 432 | SEQ ID | SEQ ID NO: 441 |
| | | 32 | | NO: 34 | |
| TBX6 | SEQ ID NO: 11 | SEQ ID NO: | SEQ ID NO: 433 | SEQ ID | SEQ ID NO: 442 |
| | | 32 | | NO: 34 | |
| TBX6 | SEQ ID NO: 11 | SEQ ID NO: | SEQ ID NO: 434 | SEQ ID | SEQ ID NO: 443 |
| | | 32 | | NO: 34 | |
| TBX6 | SEQ ID NO: 11 | SEQ ID NO: | SEQ ID NO: 435 | SEQ ID | SEQ ID NO: 444 |
| | | 32 | | NO: 34 | |
| TBX6 | SEQ ID NO: 11 | SEQ ID NO: | SEQ ID NO: 436 | SEQ ID | SEQ ID NO: 445 |
| | | 32 | | NO: 34 | |
| TBX6 | SEQ ID NO: 11 | SEQ ID NO: | SEQ ID NO: 437 | SEQ ID | SEQ ID NO: 446 |
| | | 32 | | NO: 34 | |
| TBX6 | SEQ ID NO: 11 | SEQ ID NO: | SEQ ID NO: 438 | SEQ ID | SEQ ID NO: 447 |
| | | 32 | | NO: 34 | |
| TBX6 | SEQ ID NO: 11 | SEQ ID NO: | SEQ ID NO: 439 | SEQ ID | SEQ ID NO: 448 |
| | | 32 | | NO: 34 | |
| TBX6 | SEQ ID NO: 12 | SEQ ID NO: | SEQ ID NO: 431 | SEQ ID | SEQ ID NO: 440 |
| | | 32 | | NO: 34 | |
| TBX6 | SEQ ID NO: 12 | SEQ ID NO: | SEQ ID NO: 432 | SEQ ID | SEQ ID NO: 441 |
| | | 32 | | NO: 34 | |
| TBX6 | SEQ ID NO: 12 | SEQ ID NO: | SEQ ID NO: 433 | SEQ ID | SEQ ID NO: 442 |
| | | 32 | | NO: 34 | |
| TBX6 | SEQ ID NO: 12 | SEQ ID NO: | SEQ ID NO: 434 | SEQ ID | SEQ ID NO: 443 |
| | | 32 | | NO: 34 | |
| TBX6 | SEQ ID NO: 12 | SEQ ID NO: | SEQ ID NO: 435 | SEQ ID | SEQ ID NO: 444 |
| | | 32 | | NO: 34 | |
| TBX6 | SEQ ID NO: 12 | SEQ ID NO: | SEQ ID NO: 436 | SEQ ID | SEQ ID NO: 445 |
| | | 32 | | NO: 34 | |
| TBX6 | SEQ ID NO: 12 | SEQ ID NO: | SEQ ID NO: 437 | SEQ ID | SEQ ID NO: 446 |
| | | 32 | | NO: 34 | |
| TBX6 | SEQ ID NO: 12 | SEQ ID NO: | SEQ ID NO: 438 | SEQ ID | SEQ ID NO: 447 |
| | | 32 | | NO: 34 | |
| TBX6 | SEQ ID NO: 12 | SEQ ID NO: | SEQ ID NO: 439 | SEQ ID | SEQ ID NO: 448 |
| | | 32 | | NO: 34 | |
| MSGN1 | SEQ ID NO: 15 | SEQ ID NO: | SEQ ID NO: 431 | SEQ ID | SEQ ID NO: 440 |
| | | 32 | | NO: 34 | |
| MSGN1 | SEQ ID NO: 15 | SEQ ID NO: | SEQ ID NO: 432 | SEQ ID | SEQ ID NO: 441 |
| | | 32 | | NO: 34 | |
| MSGN1 | SEQ ID NO: 15 | SEQ ID NO: | SEQ ID NO: 433 | SEQ ID | SEQ ID NO: 442 |
| | | 32 | | NO: 34 | |
| MSGN1 | SEQ ID NO: 15 | SEQ ID NO: | SEQ ID NO: 434 | SEQ ID | SEQ ID NO: 443 |
| | | 32 | | NO: 34 | |
| MSGN1 | SEQ ID NO: 15 | SEQ ID NO: | SEQ ID NO: 435 | SEQ ID | SEQ ID NO: 444 |
| | | 32 | | NO: 34 | |
| MSGN1 | SEQ ID NO: 15 | SEQ ID NO: | SEQ ID NO: 436 | SEQ ID | SEQ ID NO: 445 |
| | | 32 | | NO: 34 | |
| MSGN1 | SEQ ID NO: 15 | SEQ ID NO: | SEQ ID NO: 437 | SEQ ID | SEQ ID NO: 446 |
| | | 32 | | NO: 34 | |
| MSGN1 | SEQ ID NO: 15 | SEQ ID NO: | SEQ ID NO: 438 | SEQ ID | SEQ ID NO: 447 |
| | | 32 | | NO: 34 | |
| MSGN1 | SEQ ID NO: 15 | SEQ ID NO: | SEQ ID NO: 439 | SEQ ID | SEQ ID NO: 448 |
| | | 32 | | NO: 34 | |
| MSGN1 | SEQ ID NO: 16 | SEQ ID NO: | SEQ ID NO: 431 | SEQ ID | SEQ ID NO: 440 |
| | | 32 | | NO: 34 | |
| MSGN1 | SEQ ID NO: 16 | SEQ ID NO: | SEQ ID NO: 432 | SEQ ID | SEQ ID NO: 441 |
| | | 32 | | NO: 34 | |
| MSGN1 | SEQ ID NO: 16 | SEQ ID NO: | SEQ ID NO: 433 | SEQ ID | SEQ ID NO: 442 |
| | | 32 | | NO: 34 | |
| MSGN1 | SEQ ID NO: 16 | SEQ ID NO: | SEQ ID NO: 434 | SEQ ID | SEQ ID NO: 443 |
| | | 32 | | NO: 34 | |
| MSGN1 | SEQ ID NO: 16 | SEQ ID NO: | SEQ ID NO: 435 | SEQ ID | SEQ ID NO: 444 |
| | | 32 | | NO: 34 | |
| MSGN1 | SEQ ID NO: 16 | SEQ ID NO: | SEQ ID NO: 436 | SEQ ID | SEQ ID NO: 445 |
| | | 32 | | NO: 34 | |
| MSGN1 | SEQ ID NO: 16 | SEQ ID NO: | SEQ ID NO: 437 | SEQ ID | SEQ ID NO: 446 |
| | | 32 | | NO: 34 | |
| MSGN1 | SEQ ID NO: 16 | SEQ ID NO: | SEQ ID NO: 438 | SEQ ID | SEQ ID NO: 447 |
| | | 32 | | NO: 34 | |
| MSGN1 | SEQ ID NO: 16 | SEQ ID NO: | SEQ ID NO: 439 | SEQ ID | SEQ ID NO: 448 |
| | | 32 | | NO: 34 | |
| MSGN1 | SEQ ID NO: 17 | SEQ ID NO: | SEQ ID NO: 431 | SEQ ID | SEQ ID NO: 440 |
| | | 32 | | NO: 34 | |
| MSGN1 | SEQ ID NO: 17 | SEQ ID NO: | SEQ ID NO: 432 | SEQ ID | SEQ ID NO: 441 |
| | | 32 | | NO: 34 | |
| MSGN1 | SEQ ID NO: 17 | SEQ ID NO: | SEQ ID NO: 433 | SEQ ID | SEQ ID NO: 442 |
| | | 32 | | NO: 34 | |
| MSGN1 | SEQ ID NO: 17 | SEQ ID NO: | SEQ ID NO: 434 | SEQ ID | SEQ ID NO: 443 |
| | | 32 | | NO: 34 | |
| MSGN1 | SEQ ID NO: 17 | SEQ ID NO: | SEQ ID NO: 435 | SEQ ID | SEQ ID NO: 444 |
| | | 32 | | NO: 34 | |
| MSGN1 | SEQ ID NO: 17 | SEQ ID NO: | SEQ ID NO: 436 | SEQ ID | SEQ ID NO: 445 |
| | | 32 | | NO: 34 | |
| MSGN1 | SEQ ID NO: 17 | SEQ ID NO: | SEQ ID NO: 437 | SEQ ID | SEQ ID NO: 446 |
| | | 32 | | NO: 34 | |
| MSGN1 | SEQ ID NO: 17 | SEQ ID NO: | SEQ ID NO: 438 | SEQ ID | SEQ ID NO: 447 |
| | | 32 | | NO: 34 | |
| MSGN1 | SEQ ID NO: 17 | SEQ ID NO: | SEQ ID NO: 439 | SEQ ID | SEQ ID NO: 448 |
| | | 32 | | NO: 34 | |
| MSGN1 | SEQ ID NO: 18 | SEQ ID NO: | SEQ ID NO: 431 | SEQ ID | SEQ ID NO: 440 |
| | | 32 | | NO: 34 | |
| MSGN1 | SEQ ID NO: 18 | SEQ ID NO: | SEQ ID NO: 432 | SEQ ID | SEQ ID NO: 441 |
| | | 32 | | NO: 34 | |
| MSGN1 | SEQ ID NO: 18 | SEQ ID NO: | SEQ ID NO: 433 | SEQ ID | SEQ ID NO: 442 |
| | | 32 | | NO: 34 | |
| MSGN1 | SEQ ID NO: 18 | SEQ ID NO: | SEQ ID NO: 434 | SEQ ID | SEQ ID NO: 443 |
| | | 32 | | NO: 34 | |
| MSGN1 | SEQ ID NO: 18 | SEQ ID NO: | SEQ ID NO: 435 | SEQ ID | SEQ ID NO: 444 |
| | | 32 | | NO: 34 | |
| MSGN1 | SEQ ID NO: 18 | SEQ ID NO: | SEQ ID NO: 436 | SEQ ID | SEQ ID NO: 445 |
| | | 32 | | NO: 34 | |
| MSGN1 | SEQ ID NO: 18 | SEQ ID NO: | SEQ ID NO: 437 | SEQ ID | SEQ ID NO: 446 |
| | | 32 | | NO: 34 | |
| MSGN1 | SEQ ID NO: 18 | SEQ ID NO: | SEQ ID NO: 438 | SEQ ID | SEQ ID NO: 447 |
| | | 32 | | NO: 34 | |
| MSGN1 | SEQ ID NO: 18 | SEQ ID NO: | SEQ ID NO: 439 | SEQ ID | SEQ ID NO: 448 |
| | | 32 | | NO: 34 | |
| PAX3 | SEQ ID NO: 22 | SEQ ID NO: | SEQ ID NO: 431 | SEQ ID | SEQ ID NO: 440 |
| | | 32 | | NO: 34 | |
| PAX3 | SEQ ID NO: 22 | SEQ ID NO: | SEQ ID NO: 432 | SEQ ID | SEQ ID NO: 441 |
| | | 32 | | NO: 34 | |
| PAX3 | SEQ ID NO: 22 | SEQ ID NO: | SEQ ID NO: 433 | SEQ ID | SEQ ID NO: 442 |
| | | 32 | | NO: 34 | |
| PAX3 | SEQ ID NO: 22 | SEQ ID NO: | SEQ ID NO: 434 | SEQ ID | SEQ ID NO: 443 |
| | | 32 | | NO: 34 | |
| PAX3 | SEQ ID NO: 22 | SEQ ID NO: | SEQ ID NO: 435 | SEQ ID | SEQ ID NO: 444 |
| | | 32 | | NO: 34 | |
| PAX3 | SEQ ID NO: 22 | SEQ ID NO: | SEQ ID NO: 436 | SEQ ID | SEQ ID NO: 445 |
| | | 32 | | NO: 34 | |
| PAX3 | SEQ ID NO: 22 | SEQ ID NO: | SEQ ID NO: 437 | SEQ ID | SEQ ID NO: 446 |
| | | 32 | | NO: 34 | |
| PAX3 | SEQ ID NO: 22 | SEQ ID NO: | SEQ ID NO: 438 | SEQ ID | SEQ ID NO: 447 |
| | | 32 | | NO: 34 | |
| PAX3 | SEQ ID NO: 22 | SEQ ID NO: | SEQ ID NO: 439 | SEQ ID | SEQ ID NO: 448 |
| | | 32 | | NO: 34 | |
| PAX3 | SEQ ID NO: 23 | SEQ ID NO: | SEQ ID NO: 431 | SEQ ID | SEQ ID NO: 440 |
| | | 32 | | NO: 34 | |
| PAX3 | SEQ ID NO: 23 | SEQ ID NO: | SEQ ID NO: 432 | SEQ ID | SEQ ID NO: 441 |
| | | 32 | | NO: 34 | |
| PAX3 | SEQ ID NO: 23 | SEQ ID NO: | SEQ ID NO: 433 | SEQ ID | SEQ ID NO: 442 |
| | | 32 | | NO: 34 | |
| PAX3 | SEQ ID NO: 23 | SEQ ID NO: | SEQ ID NO: 434 | SEQ ID | SEQ ID NO: 443 |
| | | 32 | | NO: 34 | |
| PAX3 | SEQ ID NO: 23 | SEQ ID NO: | SEQ ID NO: 435 | SEQ ID | SEQ ID NO: 444 |
| | | 32 | | NO: 34 | |
| PAX3 | SEQ ID NO: 23 | SEQ ID NO: | SEQ ID NO: 436 | SEQ ID | SEQ ID NO: 445 |
| | | 32 | | NO: 34 | |
| PAX3 | SEQ ID NO: 23 | SEQ ID NO: | SEQ ID NO: 437 | SEQ ID | SEQ ID NO: 446 |
| | | 32 | | NO: 34 | |
| PAX3 | SEQ ID NO: 23 | SEQ ID NO: | SEQ ID NO: 438 | SEQ ID | SEQ ID NO: 447 |
| | | 32 | | NO: 34 | |
| PAX3 | SEQ ID NO: 23 | SEQ ID NO: | SEQ ID NO: 439 | SEQ ID | SEQ ID NO: 448 |
| | | 32 | | NO: 34 | |
| PAX3 | SEQ ID NO: 24 | SEQ ID NO: | SEQ ID NO: 431 | SEQ ID | SEQ ID NO: 440 |
| | | 32 | | NO: 34 | |
| PAX3 | SEQ ID NO: 24 | SEQ ID NO: | SEQ ID NO: 432 | SEQ ID | SEQ ID NO: 441 |
| | | 32 | | NO: 34 | |
| PAX3 | SEQ ID NO: 24 | SEQ ID NO: | SEQ ID NO: 433 | SEQ ID | SEQ ID NO: 442 |
| | | 32 | | NO: 34 | |
| PAX3 | SEQ ID NO: 24 | SEQ ID NO: | SEQ ID NO: 434 | SEQ ID | SEQ ID NO: 443 |
| | | 32 | | NO: 34 | |
| PAX3 | SEQ ID NO: 24 | SEQ ID NO: | SEQ ID NO: 435 | SEQ ID | SEQ ID NO: 444 |
| | | 32 | | NO: 34 | |
| PAX3 | SEQ ID NO: 24 | SEQ ID NO: | SEQ ID NO: 436 | SEQ ID | SEQ ID NO: 445 |
| | | 32 | | NO: 34 | |
| PAX3 | SEQ ID NO: 24 | SEQ ID NO: | SEQ ID NO: 437 | SEQ ID | SEQ ID NO: 446 |
| | | 32 | | NO: 34 | |
| PAX3 | SEQ ID NO: 24 | SEQ ID NO: | SEQ ID NO: 438 | SEQ ID | SEQ ID NO: 447 |
| | | 32 | | NO: 34 | |
| PAX3 | SEQ ID NO: 24 | SEQ ID NO: | SEQ ID NO: 439 | SEQ ID | SEQ ID NO: 448 |
| | | 32 | | NO: 34 | |
| PAX3 | SEQ ID NO: 25 | SEQ ID NO: | SEQ ID NO: 431 | SEQ ID | SEQ ID NO: 440 |
| | | 32 | | NO: 34 | |
| PAX3 | SEQ ID NO: 25 | SEQ ID NO: | SEQ ID NO: 432 | SEQ ID | SEQ ID NO: 441 |
| | | 32 | | NO: 34 | |
| PAX3 | SEQ ID NO: 25 | SEQ ID NO: | SEQ ID NO: 433 | SEQ ID | SEQ ID NO: 442 |
| | | 32 | | NO: 34 | |
| PAX3 | SEQ ID NO: 25 | SEQ ID NO: | SEQ ID NO: 434 | SEQ ID | SEQ ID NO: 443 |
| | | 32 | | NO: 34 | |
| PAX3 | SEQ ID NO: 25 | SEQ ID NO: | SEQ ID NO: 435 | SEQ ID | SEQ ID NO: 444 |
| | | 32 | | NO: 34 | |
| PAX3 | SEQ ID NO: 25 | SEQ ID NO: | SEQ ID NO: 436 | SEQ ID | SEQ ID NO: 445 |
| | | 32 | | NO: 34 | |
| PAX3 | SEQ ID NO: 25 | SEQ ID NO: | SEQ ID NO: 437 | SEQ ID | SEQ ID NO: 446 |
| | | 32 | | NO: 34 | |
| PAX3 | SEQ ID NO: 25 | SEQ ID NO: | SEQ ID NO: 438 | SEQ ID | SEQ ID NO: 447 |
| | | 32 | | NO: 34 | |
| PAX3 | SEQ ID NO: 25 | SEQ ID NO: | SEQ ID NO: 439 | SEQ ID | SEQ ID NO: 448 |
| | | 32 | | NO: 34 | |
| PAX7 | SEQ ID NO: 28 | SEQ ID NO: | SEQ ID NO: 431 | SEQ ID | SEQ ID NO: 440 |
| | | 32 | | NO: 34 | |
| PAX7 | SEQ ID NO: 28 | SEQ ID NO: | SEQ ID NO: 432 | SEQ ID | SEQ ID NO: 441 |
| | | 32 | | NO: 34 | |
| PAX7 | SEQ ID NO: 28 | SEQ ID NO: | SEQ ID NO: 433 | SEQ ID | SEQ ID NO: 442 |
| | | 32 | | NO: 34 | |
| PAX7 | SEQ ID NO: 28 | SEQ ID NO: | SEQ ID NO: 434 | SEQ ID | SEQ ID NO: 443 |
| | | 32 | | NO: 34 | |
| PAX7 | SEQ ID NO: 28 | SEQ ID NO: | SEQ ID NO: 435 | SEQ ID | SEQ ID NO: 444 |
| | | 32 | | NO: 34 | |
| PAX7 | SEQ ID NO: 28 | SEQ ID NO: | SEQ ID NO: 436 | SEQ ID | SEQ ID NO: 445 |
| | | 32 | | NO: 34 | |
| PAX7 | SEQ ID NO: 28 | SEQ ID NO: | SEQ ID NO: 437 | SEQ ID | SEQ ID NO: 446 |
| | | 32 | | NO: 34 | |
| PAX7 | SEQ ID NO: 28 | SEQ ID NO: | SEQ ID NO: 438 | SEQ ID | SEQ ID NO: 447 |
| | | 32 | | NO: 34 | |
| PAX7 | SEQ ID NO: 28 | SEQ ID NO: | SEQ ID NO: 439 | SEQ ID | SEQ ID NO: 448 |
| | | 32 | | NO: 34 | |
| PAX7 | SEQ ID NO: 29 | SEQ ID NO: | SEQ ID NO: 431 | SEQ ID | SEQ ID NO: 440 |
| | | 32 | | NO: 34 | |
| PAX7 | SEQ ID NO: 29 | SEQ ID NO: | SEQ ID NO: 432 | SEQ ID | SEQ ID NO: 441 |
| | | 32 | | NO: 34 | |
| PAX7 | SEQ ID NO: 29 | SEQ ID NO: | SEQ ID NO: 433 | SEQ ID | SEQ ID NO: 442 |
| | | 32 | | NO: 34 | |
| PAX7 | SEQ ID NO: 29 | SEQ ID NO: | SEQ ID NO: 434 | SEQ ID | SEQ ID NO: 443 |
| | | 32 | | NO: 34 | |
| PAX7 | SEQ ID NO: 29 | SEQ ID NO: | SEQ ID NO: 435 | SEQ ID | SEQ ID NO: 444 |
| | | 32 | | NO: 34 | |
| PAX7 | SEQ ID NO: 29 | SEQ ID NO: | SEQ ID NO: 436 | SEQ ID | SEQ ID NO: 445 |
| | | 32 | | NO: 34 | |
| PAX7 | SEQ ID NO: 29 | SEQ ID NO: | SEQ ID NO: 437 | SEQ ID | SEQ ID NO: 446 |
| | | 32 | | NO: 34 | |
| PAX7 | SEQ ID NO: 29 | SEQ ID NO: | SEQ ID NO: 438 | SEQ ID | SEQ ID NO: 447 |
| | | 32 | | NO: 34 | |
| PAX7 | SEQ ID NO: 29 | SEQ ID NO: | SEQ ID NO: 439 | SEQ ID | SEQ ID NO: 448 |
| | | 32 | | NO: 34 | |
| PAX7 | SEQ ID NO: 30 | SEQ ID NO: | SEQ ID NO: 431 | SEQ ID | SEQ ID NO: 440 |
| | GGCCCATTC | 32 | | NO: 34 | |
| | ATCAA | | | | |
| PAX7 | SEQ ID NO: 30 | SEQ ID NO: | SEQ ID NO: 432 | SEQ ID | SEQ ID NO: 441 |
| | | 32 | | NO: 34 | |
| PAX7 | SEQ ID NO: 30 | SEQ ID NO: | SEQ ID NO: 433 | SEQ ID | SEQ ID NO: 442 |
| | | 32 | | NO: 34 | |
| PAX7 | SEQ ID NO: 30 | SEQ ID NO: | SEQ ID NO: 434 | SEQ ID | SEQ ID NO: 443 |
| | | 32 | | NO: 34 | |
| PAX7 | SEQ ID NO: 30 | SEQ ID NO: | SEQ ID NO: 435 | SEQ ID | SEQ ID NO: 444 |
| | | 32 | | NO: 34 | |
| PAX7 | SEQ ID NO: 30 | SEQ ID NO: | SEQ ID NO: 436 | SEQ ID | SEQ ID NO: 445 |
| | | 32 | | NO: 34 | |
| PAX7 | SEQ ID NO: 30 | SEQ ID NO: | SEQ ID NO: 437 | SEQ ID | SEQ ID NO: 446 |
| | | 32 | | NO: 34 | |
| PAX7 | SEQ ID NO: 30 | SEQ ID NO: | SEQ ID NO: 438 | SEQ ID | SEQ ID NO: 447 |
| | | 32 | | NO: 34 | |
| PAX7 | SEQ ID NO: 30 | SEQ ID NO: | SEQ ID NO: 439 | SEQ ID | SEQ ID NO: 448 |
| | | 32 | | NO: 34 | |
| PAX7 | SEQ ID NO: 31 | SEQ ID NO: | SEQ ID NO: 431 | SEQ ID | SEQ ID NO: 440 |
| | | 32 | | NO: 34 | |
| PAX7 | SEQ ID NO: 31 | SEQ ID NO: | SEQ ID NO: 432 | SEQ ID | SEQ ID NO: 441 |
| | | 32 | | NO: 34 | |
| PAX7 | SEQ ID NO: 31 | SEQ ID NO: | SEQ ID NO: 433 | SEQ ID | SEQ ID NO: 442 |
| | | 32 | | NO: 34 | |
| PAX7 | SEQ ID NO: 31 | SEQ ID NO: | SEQ ID NO: 434 | SEQ ID | SEQ ID NO: 443 |
| | | 32 | | NO: 34 | |
| PAX7 | SEQ ID NO: 31 | SEQ ID NO: | SEQ ID NO: 435 | SEQ ID | SEQ ID NO: 444 |
| | | 32 | | NO: 34 | |
| PAX7 | SEQ ID NO: 31 | SEQ ID NO: | SEQ ID NO: 436 | SEQ ID | SEQ ID NO: 445 |
| | | 32 | | NO: 34 | |
| PAX7 | SEQ ID NO: 31 | SEQ ID NO: | SEQ ID NO: 437 | SEQ ID | SEQ ID NO: 446 |
| | | 32 | | NO: 34 | |
| PAX7 | SEQ ID NO: 31 | SEQ ID NO: | SEQ ID NO: 438 | SEQ ID | SEQ ID NO: 447 |
| | | 32 | | NO: 34 | |
| PAX7 | SEQ ID NO: 31 | SEQ ID NO: | SEQ ID NO: 439 | SEQ ID | SEQ ID NO: 448 |
| | | 32 | | NO: 34 | |

TABLE 5
Concatenated portion of the proGuide from Table 4

| GENE_ | SEQ ID | Concatenated portion of a proGuide (SPACER + EXTRA + |
|---|---|---|
| TARGET | NO: | UPSTREAM STEM + POLYT_UNIT + DOWNSTREAM STEM) |
| TBXT | 101 | GCTTTCACGGAAGGGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBXT | 102 | CTGCGCATCGGGTGGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBXT | 103 | CCGCACTTCCGCGAGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBXT | 104 | GCGCCAAGCGGTGTGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBXT | 105 | GCTTTCACGGAAGGGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| TBXT | 106 | CTGCGCATCGGGTGGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| TBXT | 107 | CCGCACTTCCGCGAGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| TBXT | 108 | GCGCCAAGCGGTGTGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| TBXT | 109 | GCTTTCACGGAAGGGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBXT | 110 | CTGCGCATCGGGTGGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBXT | 111 | CCGCACTTCCGCGAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBXT | 112 | GCGCCAAGCGGTGTGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBX6 | 113 | CGAGGTCACGGCGTGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBX6 | 114 | CCGCGTGGTCCGTCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBX6 | 115 | GGGGCAGCGTCCCAGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBX6 | 116 | CCAGACCTTCTGGCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| TBX6 | 117 | CGAGGTCACGGCGTGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| TBX6 | 118 | CCGCGTGGTCCGTCGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| TBX6 | 119 | GGGGCAGCGTCCCAGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| TBX6 | 120 | CCAGACCTTCTGGCGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| TBX6 | 121 | CGAGGTCACGGCGTGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBX6 | 122 | CCGCGTGGTCCGTCGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBX6 | 123 | GGGGCAGCGTCCCAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| TBX6 | 124 | CCAGACCTTCTGGCGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| MSGN1 | 125 | TATGCAGCCTTAATGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| MSGN1 | 126 | CTGCCGCCTGGCCAGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| MSGN1 | 127 | AAAGTGACCCGAGAGTTTTAGAGCTACCTTCCCGACTTCCGTAGG |
| | | AGCGCTTTTTTTTcagccaactccaaTTTTTTTTGCGCTCCTACGGAAGTCG |
| | | GGAAGG |
| MSGN1 | 128 | GCTGCGGATGCCCCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| MSGN1 | 129 | TATGCAGCCTTAATGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| MSGN1 | 130 | CTGCCGCCTGGCCAGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| MSGN1 | 131 | AAAGTGACCCGAGAGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| MSGN1 | 132 | GCTGCGGATGCCCCGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| MSGN1 | 133 | TATGCAGCCTTAATGTTTTAGAGCTACCTGAAGGTAAAGATCGGGT |
| | | CCTCTTTTTTTTcagccaactccaaTTTTTTTTGAGGACCCGATCTTTACCT |
| | | TCAGG |
| MSGN1 | 134 | CTGCCGCCTGGCCAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| MSGN1 | 135 | AAAGTGACCCGAGAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| MSGN1 | 136 | GCTGCGGATGCCCCGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX3 | 137 | GGGTAGACCGTCCCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX3 | 138 | CGAGTGCGGGGATCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX3 | 139 | GCACCACCAGAGATGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX3 | 140 | CAACCTTCTCTCCTGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX3 | 141 | GGGTAGACCGTCCCGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| PAX3 | 142 | CGAGTGCGGGGATCGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| PAX3 | 143 | GCACCACCAGAGATGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| PAX3 | 144 | CAACCTTCTCTCCTGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| PAX3 | 145 | GGGTAGACCGTCCCGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX3 | 146 | CGAGTGCGGGGATCGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX3 | 147 | GCACCACCAGAGATGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX3 | 148 | CAACCTTCTCTCCTGTTTTAGAGCTACCTGAAGGTAAAGATCGGGT |
| | | CCTCTTTTTTTTcagccaactccaaTTTTTTTTGAGGACCCGATCTTTACCT |
| | | TCAGG |
| PAX7 | 149 | TAGGGTGACTTGAAGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX7 | 150 | ACGAGGTCGAGCCGGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX7 | 151 | GGCCCATTCATCAAGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX7 | 152 | GCTCCCTTGCGCCCGTTTTAGAGCTACCTTCCCGACTTCCGTAGGA |
| | | GCGCTTTTTTTTcagccaactccaaTTTTTTTTGCGCTCCTACGGAAGTCGG |
| | | GAAGG |
| PAX7 | 153 | TAGGGTGACTTGAAGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| PAX7 | 154 | ACGAGGTCGAGCCGGTTTTAGAGCTACCGGTGCTCCGCATACCAA |
| | | GCCGTTTTTTTTcagccaactccaaTTTTTTTTCGGCTTGGTATGCGGAGCA |
| | | CCGG |
| PAX7 | 155 | GGCCCATTCATCAAGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| PAX7 | 156 | GCTCCCTTGCGCCCGTTTTAGAGCTACCGGTGCTCCGCATACCAAG |
| | | CCGTTTTTTTTcagccaactccaaTTTTTTTTCGGCTTGGTATGCGGAGCAC |
| | | CGG |
| PAX7 | 157 | TAGGGTGACTTGAAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX7 | 158 | ACGAGGTCGAGCCGGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX7 | 159 | GGCCCATTCATCAAGTTTTAGAGCTACCTGAAGGTAAAGATCGGG |
| | | TCCTCTTTTTTTTcagccaactccaaTTTTTTTTGAGGACCCGATCTTTACC |
| | | TTCAGG |
| PAX7 | 160 | GCTCCCTTGCGCCCGTTTTAGAGCTACCTGAAGGTAAAGATCGGGT |
| | | CCTCTTTTTTTTcagccaactccaaTTTTTTTTGAGGACCCGATCTTTACCT |
| | | TCAGG |
| TBXT | 161 | GCTTTCACGGAAGGGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBXT | 162 | GCTTTCACGGAAGGGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBXT | 163 | GCTTTCACGGAAGGGTTTTAGAGCTAGCGCACGACCACTATCGTGT |
| | | AGGTTTTTTTTcagccaactccaaTTTTTTTTCCTACACGATAGTGGTCGTG |
| | | CGC |
| TBXT | 164 | GCTTTCACGGAAGGGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBXT | 165 | GCTTTCACGGAAGGGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBXT | 166 | GCTTTCACGGAAGGGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| TBXT | 167 | GCTTTCACGGAAGGGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBXT | 168 | GCTTTCACGGAAGGGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBXT | 169 | GCTTTCACGGAAGGGTTTTAGAGCTATGCTACGCATACGTGACGA |
| | | CGGGTTTTTTTTcagccaactccaaTTTTTTTTCCCGTCGTCACGTATGCGT |
| | | AGCA |
| TBXT | 170 | CTGCGCATCGGGTGGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBXT | 171 | CTGCGCATCGGGTGGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBXT | 172 | CTGCGCATCGGGTGGTTTTAGAGCTAGCGCACGACCACTATCGTGT |
| | | AGGTTTTTTTTcagccaactccaaTTTTTTTTCCTACACGATAGTGGTCGTG |
| | | CGC |
| TBXT | 173 | CTGCGCATCGGGTGGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBXT | 174 | CTGCGCATCGGGTGGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBXT | 175 | CTGCGCATCGGGTGGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| TBXT | 176 | CTGCGCATCGGGTGGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBXT | 177 | CTGCGCATCGGGTGGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBXT | 178 | CTGCGCATCGGGTGGTTTTAGAGCTATGCTACGCATACGTGACGAC |
| | | GGGTTTTTTTTcagccaactccaaTTTTTTTTCCCGTCGTCACGTATGCGTA |
| | | GCA |
| TBXT | 179 | CCGCACTTCCGCGAGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBXT | 180 | CCGCACTTCCGCGAGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBXT | 181 | CCGCACTTCCGCGAGTTTTAGAGCTAGCGCACGACCACTATCGTGT |
| | | AGGTTTTTTTTcagccaactccaaTTTTTTTTCCTACACGATAGTGGTCGTG |
| | | CGC |
| TBXT | 182 | CCGCACTTCCGCGAGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBXT | 183 | CCGCACTTCCGCGAGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBXT | 184 | CCGCACTTCCGCGAGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| TBXT | 185 | CCGCACTTCCGCGAGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBXT | 186 | CCGCACTTCCGCGAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBXT | 187 | CCGCACTTCCGCGAGTTTTAGAGCTATGCTACGCATACGTGACGAC |
| | | GGGTTTTTTTTcagccaactccaaTTTTTTTTCCCGTCGTCACGTATGCGTA |
| | | GCA |
| TBXT | 188 | GCGCCAAGCGGTGTGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBXT | 189 | GCGCCAAGCGGTGTGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBXT | 190 | GCGCCAAGCGGTGTGTTTTAGAGCTAGCGCACGACCACTATCGTG |
| | | TAGGTTTTTTTTcagccaactccaaTTTTTTTTCCTACACGATAGTGGTCGT |
| | | GCGC |
| TBXT | 191 | GCGCCAAGCGGTGTGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBXT | 192 | GCGCCAAGCGGTGTGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBXT | 193 | GCGCCAAGCGGTGTGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| TBXT | 194 | GCGCCAAGCGGTGTGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBXT | 195 | GCGCCAAGCGGTGTGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBXT | 196 | GCGCCAAGCGGTGTGTTTTAGAGCTATGCTACGCATACGTGACGA |
| | | CGGGTTTTTTTTcagccaactccaaTTTTTTTTCCCGTCGTCACGTATGCGT |
| | | AGCA |
| TBX6 | 197 | CGAGGTCACGGCGTGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBX6 | 198 | CGAGGTCACGGCGTGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBX6 | 199 | CGAGGTCACGGCGTGTTTTAGAGCTAGCGCACGACCACTATCGTG |
| | | TAGGTTTTTTTTcagccaactccaaTTTTTTTTCCTACACGATAGTGGTCGT |
| | | GCGC |
| TBX6 | 200 | CGAGGTCACGGCGTGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBX6 | 201 | CGAGGTCACGGCGTGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBX6 | 202 | CGAGGTCACGGCGTGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| TBX6 | 203 | CGAGGTCACGGCGTGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBX6 | 204 | CGAGGTCACGGCGTGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBX6 | 205 | CGAGGTCACGGCGTGTTTTAGAGCTATGCTACGCATACGTGACGA |
| | | CGGGTTTTTTTTcagccaactccaaTTTTTTTTCCCGTCGTCACGTATGCGT |
| | | AGCA |
| TBX6 | 206 | CCGCGTGGTCCGTCGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBX6 | 207 | CCGCGTGGTCCGTCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBX6 | 208 | CCGCGTGGTCCGTCGTTTTAGAGCTAGCGCACGACCACTATCGTGT |
| | | AGGTTTTTTTTcagccaactccaaTTTTTTTTCCTACACGATAGTGGTCGTG |
| | | CGC |
| TBX6 | 209 | CCGCGTGGTCCGTCGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBX6 | 210 | CCGCGTGGTCCGTCGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBX6 | 211 | CCGCGTGGTCCGTCGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| TBX6 | 212 | CCGCGTGGTCCGTCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBX6 | 213 | CCGCGTGGTCCGTCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBX6 | 214 | CCGCGTGGTCCGTCGTTTTAGAGCTATGCTACGCATACGTGACGAC |
| | | GGGTTTTTTTTcagccaactccaaTTTTTTTTCCCGTCGTCACGTATGCGTA |
| | | GCA |
| TBX6 | 215 | GGGGCAGCGTCCCAGTTTTAGAGCTATAGCTACCGATGTCGAGTG |
| | | TAGGTTTTTTTTcagccaactccaaTTTTTTTTCCTACACTCGACATCGGTA |
| | | GCTA |
| TBX6 | 216 | GGGGCAGCGTCCCAGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBX6 | 217 | GGGGCAGCGTCCCAGTTTTAGAGCTAGCGCACGACCACTATCGTG |
| | | TAGGTTTTTTTTcagccaactccaaTTTTTTTTCCTACACGATAGTGGTCGT |
| | | GCGC |
| TBX6 | 218 | GGGGCAGCGTCCCAGTTTTAGAGCTAACTCGTTCGATAGAGAGTT |
| | | CGGGTTTTTTTTcagccaactccaaTTTTTTTTCCCGAACTCTCTATCGAAC |
| | | GAGT |
| TBX6 | 219 | GGGGCAGCGTCCCAGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBX6 | 220 | GGGGCAGCGTCCCAGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| TBX6 | 221 | GGGGCAGCGTCCCAGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBX6 | 222 | GGGGCAGCGTCCCAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBX6 | 223 | GGGGCAGCGTCCCAGTTTTAGAGCTATGCTACGCATACGTGACGA |
| | | CGGGTTTTTTTTcagccaactccaaTTTTTTTTCCCGTCGTCACGTATGCGT |
| | | AGCA |
| TBX6 | 224 | CCAGACCTTCTGGCGTTTTAGAGCTATAGCTACCGATGTCGAGTGT |
| | | AGGTTTTTTTTcagccaactccaaTTTTTTTTCCTACACTCGACATCGGTAG |
| | | CTA |
| TBX6 | 225 | CCAGACCTTCTGGCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| TBX6 | 226 | CCAGACCTTCTGGCGTTTTAGAGCTAGCGCACGACCACTATCGTGT |
| | | AGGTTTTTTTTcagccaactccaaTTTTTTTTCCTACACGATAGTGGTCGTG |
| | | CGC |
| TBX6 | 227 | CCAGACCTTCTGGCGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| TBX6 | 228 | CCAGACCTTCTGGCGTTTTAGAGCTACCTCCGTGTCGATCGTGCCA |
| | | TGGTTTTTTTTcagccaactccaaTTTTTTTTCCATGGCACGATCGACACGG |
| | | AGG |
| TBX6 | 229 | CCAGACCTTCTGGCGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| TBX6 | 230 | CCAGACCTTCTGGCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| TBX6 | 231 | CCAGACCTTCTGGCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| TBX6 | 232 | CCAGACCTTCTGGCGTTTTAGAGCTATGCTACGCATACGTGACGAC |
| | | GGGTTTTTTTTcagccaactccaaTTTTTTTTCCCGTCGTCACGTATGCGTA |
| | | GCA |
| MSGN1 | 233 | TATGCAGCCTTAATGTTTTAGAGCTATAGCTACCGATGTCGAGTGT |
| | | AGGTTTTTTTTcagccaactccaaTTTTTTTTCCTACACTCGACATCGGTAG |
| | | CTA |
| MSGN1 | 234 | TATGCAGCCTTAATGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| MSGN1 | 235 | TATGCAGCCTTAATGTTTTAGAGCTAGCGCACGACCACTATCGTGT |
| | | AGGTTTTTTTTcagccaactccaaTTTTTTTTCCTACACGATAGTGGTCGTG |
| | | CGC |
| MSGN1 | 236 | TATGCAGCCTTAATGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| MSGN1 | 237 | TATGCAGCCTTAATGTTTTAGAGCTACCTCCGTGTCGATCGTGCCA |
| | | TGGTTTTTTTTcagccaactccaaTTTTTTTTCCATGGCACGATCGACACGG |
| | | AGG |
| MSGN1 | 238 | TATGCAGCCTTAATGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| MSGN1 | 239 | TATGCAGCCTTAATGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| MSGN1 | 240 | TATGCAGCCTTAATGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| MSGN1 | 241 | TATGCAGCCTTAATGTTTTAGAGCTATGCTACGCATACGTGACGAC |
| | | GGGTTTTTTTTcagccaactccaaTTTTTTTTCCCGTCGTCACGTATGCGTA |
| | | GCA |
| MSGN1 | 242 | CTGCCGCCTGGCCAGTTTTAGAGCTATAGCTACCGATGTCGAGTGT |
| | | AGGTTTTTTTTcagccaactccaaTTTTTTTTCCTACACTCGACATCGGTAG |
| | | CTA |
| MSGN1 | 243 | CTGCCGCCTGGCCAGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| MSGN1 | 244 | CTGCCGCCTGGCCAGTTTTAGAGCTAGCGCACGACCACTATCGTGT |
| | | AGGTTTTTTTTcagccaactccaaTTTTTTTTCCTACACGATAGTGGTCGTG |
| | | CGC |
| MSGN1 | 245 | CTGCCGCCTGGCCAGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| MSGN1 | 246 | CTGCCGCCTGGCCAGTTTTAGAGCTACCTCCGTGTCGATCGTGCCA |
| | | TGGTTTTTTTTcagccaactccaaTTTTTTTTCCATGGCACGATCGACACGG |
| | | AGG |
| MSGN1 | 247 | CTGCCGCCTGGCCAGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| MSGN1 | 248 | CTGCCGCCTGGCCAGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| MSGN1 | 249 | CTGCCGCCTGGCCAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| MSGN1 | 250 | CTGCCGCCTGGCCAGTTTTAGAGCTATGCTACGCATACGTGACGAC |
| | | GGGTTTTTTTTcagccaactccaaTTTTTTTTCCCGTCGTCACGTATGCGTA |
| | | GCA |
| MSGN1 | 251 | AAAGTGACCCGAGAGTTTTAGAGCTATAGCTACCGATGTCGAGTG |
| | | TAGGTTTTTTTTcagccaactccaaTTTTTTTTCCTACACTCGACATCGGTA |
| | | GCTA |
| MSGN1 | 252 | AAAGTGACCCGAGAGTTTTAGAGCTAATTACTCGAACGTTCCGCC |
| | | AGGGTTTTTTTTcagccaactccaaTTTTTTTTCCCTGGCGGAACGTTCGA |
| | | GTAAT |
| MSGN1 | 253 | AAAGTGACCCGAGAGTTTTAGAGCTAGCGCACGACCACTATCGTG |
| | | TAGGTTTTTTTTcagccaactccaaTTTTTTTTCCTACACGATAGTGGTCGT |
| | | GCGC |
| MSGN1 | 254 | AAAGTGACCCGAGAGTTTTAGAGCTAACTCGTTCGATAGAGAGTT |
| | | CGGGTTTTTTTTcagccaactccaaTTTTTTTTCCCGAACTCTCTATCGAAC |
| | | GAGT |
| MSGN1 | 255 | AAAGTGACCCGAGAGTTTTAGAGCTACCTCCGTGTCGATCGTGCC |
| | | ATGGTTTTTTTTcagccaactccaaTTTTTTTTCCATGGCACGATCGACACG |
| | | GAGG |
| MSGN1 | 256 | AAAGTGACCCGAGAGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| MSGN1 | 257 | AAAGTGACCCGAGAGTTTTAGAGCTATAGCTCCCGTCCGTAGACG |
| | | TCGGTTTTTTTTcagccaactccaaTTTTTTTTCCGACGTCTACGGACGGGA |
| | | GCTA |
| MSGN1 | 258 | AAAGTGACCCGAGAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| MSGN1 | 259 | AAAGTGACCCGAGAGTTTTAGAGCTATGCTACGCATACGTGACGA |
| | | CGGGTTTTTTTTcagccaactccaaTTTTTTTTCCCGTCGTCACGTATGCGT |
| | | AGCA |
| MSGN1 | 260 | GCTGCGGATGCCCCGTTTTAGAGCTATAGCTACCGATGTCGAGTGT |
| | | AGGTTTTTTTTcagccaactccaaTTTTTTTTCCTACACTCGACATCGGTAG |
| | | CTA |
| MSGN1 | 261 | GCTGCGGATGCCCCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| MSGN1 | 262 | GCTGCGGATGCCCCGTTTTAGAGCTAGCGCACGACCACTATCGTGT |
| | | AGGTTTTTTTTcagccaactccaaTTTTTTTTCCTACACGATAGTGGTCGTG |
| | | CGC |
| MSGN1 | 263 | GCTGCGGATGCCCCGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| MSGN1 | 264 | GCTGCGGATGCCCCGTTTTAGAGCTACCTCCGTGTCGATCGTGCCA |
| | | TGGTTTTTTTTcagccaactccaaTTTTTTTTCCATGGCACGATCGACACGG |
| | | AGG |
| MSGN1 | 265 | GCTGCGGATGCCCCGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| MSGN1 | 266 | GCTGCGGATGCCCCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| MSGN1 | 267 | GCTGCGGATGCCCCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| MSGN1 | 268 | GCTGCGGATGCCCCGTTTTAGAGCTATGCTACGCATACGTGACGAC |
| | | GGGTTTTTTTTcagccaactccaaTTTTTTTTCCCGTCGTCACGTATGCGTA |
| | | GCA |
| PAX3 | 269 | GGGTAGACCGTCCCGTTTTAGAGCTATAGCTACCGATGTCGAGTGT |
| | | AGGTTTTTTTTcagccaactccaaTTTTTTTTCCTACACTCGACATCGGTAG |
| | | CTA |
| PAX3 | 270 | GGGTAGACCGTCCCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX3 | 271 | GGGTAGACCGTCCCGTTTTAGAGCTAGCGCACGACCACTATCGTGT |
| | | AGGTTTTTTTTcagccaactccaaTTTTTTTTCCTACACGATAGTGGTCGTG |
| | | CGC |
| PAX3 | 272 | GGGTAGACCGTCCCGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| PAX3 | 273 | GGGTAGACCGTCCCGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX3 | 274 | GGGTAGACCGTCCCGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| PAX3 | 275 | GGGTAGACCGTCCCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX3 | 276 | GGGTAGACCGTCCCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX3 | 277 | GGGTAGACCGTCCCGTTTTAGAGCTATGCTACGCATACGTGACGA |
| | | CGGGTTTTTTTTcagccaactccaaTTTTTTTTCCCGTCGTCACGTATGCGT |
| | | AGCA |
| PAX3 | 278 | CGAGTGCGGGGATCGTTTTAGAGCTATAGCTACCGATGTCGAGTG |
| | | TAGGTTTTTTTTcagccaactccaaTTTTTTTTCCTACACTCGACATCGGTA |
| | | GCTA |
| PAX3 | 279 | CGAGTGCGGGGATCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX3 | 280 | CGAGTGCGGGGATCGTTTTAGAGCTAGCGCACGACCACTATCGTG |
| | | TAGGTTTTTTTTcagccaactccaaTTTTTTTTCCTACACGATAGTGGTCGT |
| | | GCGC |
| PAX3 | 281 | CGAGTGCGGGGATCGTTTTAGAGCTAACTCGTTCGATAGAGAGTT |
| | | CGGGTTTTTTTTcagccaactccaaTTTTTTTTCCCGAACTCTCTATCGAAC |
| | | GAGT |
| PAX3 | 282 | CGAGTGCGGGGATCGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX3 | 283 | CGAGTGCGGGGATCGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| PAX3 | 284 | CGAGTGCGGGGATCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX3 | 285 | CGAGTGCGGGGATCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX3 | 286 | CGAGTGCGGGGATCGTTTTAGAGCTATGCTACGCATACGTGACGA |
| | | CGGGTTTTTTTTcagccaactccaaTTTTTTTTCCCGTCGTCACGTATGCGT |
| | | AGCA |
| PAX3 | 287 | GCACCACCAGAGATGTTTTAGAGCTATAGCTACCGATGTCGAGTG |
| | | TAGGTTTTTTTTcagccaactccaaTTTTTTTTCCTACACTCGACATCGGTA |
| | | GCTA |
| PAX3 | 288 | GCACCACCAGAGATGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX3 | 289 | GCACCACCAGAGATGTTTTAGAGCTAGCGCACGACCACTATCGTG |
| | | TAGGTTTTTTTTcagccaactccaaTTTTTTTTCCTACACGATAGTGGTCGT |
| | | GCGC |
| PAX3 | 290 | GCACCACCAGAGATGTTTTAGAGCTAACTCGTTCGATAGAGAGTT |
| | | CGGGTTTTTTTTcagccaactccaaTTTTTTTTCCCGAACTCTCTATCGAAC |
| | | GAGT |
| PAX3 | 291 | GCACCACCAGAGATGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX3 | 292 | GCACCACCAGAGATGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| PAX3 | 293 | GCACCACCAGAGATGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX3 | 294 | GCACCACCAGAGATGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX3 | 295 | GCACCACCAGAGATGTTTTAGAGCTATGCTACGCATACGTGACGA |
| | | CGGGTTTTTTTTcagccaactccaaTTTTTTTTCCCGTCGTCACGTATGCGT |
| | | AGCA |
| PAX3 | 296 | CAACCTTCTCTCCTGTTTTAGAGCTATAGCTACCGATGTCGAGTGT |
| | | AGGTTTTTTTTcagccaactccaaTTTTTTTTCCTACACTCGACATCGGTAG |
| | | CTA |
| PAX3 | 297 | CAACCTTCTCTCCTGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX3 | 298 | CAACCTTCTCTCCTGTTTTAGAGCTAGCGCACGACCACTATCGTGT |
| | | AGGTTTTTTTTcagccaactccaaTTTTTTTTCCTACACGATAGTGGTCGTG |
| | | CGC |
| PAX3 | 299 | CAACCTTCTCTCCTGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| PAX3 | 300 | CAACCTTCTCTCCTGTTTTAGAGCTACCTCCGTGTCGATCGTGCCAT |
| | | GGTTTTTTTTcagccaactccaaTTTTTTTTCCATGGCACGATCGACACGG |
| | | AGG |
| PAX3 | 301 | CAACCTTCTCTCCTGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| PAX3 | 302 | CAACCTTCTCTCCTGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX3 | 303 | CAACCTTCTCTCCTGTTTTAGAGCTATGCGTCGTCTACTACCTCTCG |
| | | GGTTTTTTTTcagccaactccaaTTTTTTTTCCCGAGAGGTAGTAGACGAC |
| | | GCA |
| PAX3 | 304 | CAACCTTCTCTCCTGTTTTAGAGCTATGCTACGCATACGTGACGAC |
| | | GGGTTTTTTTTcagccaactccaaTTTTTTTTCCCGTCGTCACGTATGCGTA |
| | | GCA |
| PAX7 | 305 | TAGGGTGACTTGAAGTTTTAGAGCTATAGCTACCGATGTCGAGTGT |
| | | AGGTTTTTTTTcagccaactccaaTTTTTTTTCCTACACTCGACATCGGTAG |
| | | CTA |
| PAX7 | 306 | TAGGGTGACTTGAAGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX7 | 307 | TAGGGTGACTTGAAGTTTTAGAGCTAGCGCACGACCACTATCGTGT |
| | | AGGTTTTTTTTcagccaactccaaTTTTTTTTCCTACACGATAGTGGTCGTG |
| | | CGC |
| PAX7 | 308 | TAGGGTGACTTGAAGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| PAX7 | 309 | TAGGGTGACTTGAAGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX7 | 400 | TAGGGTGACTTGAAGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| PAX7 | 401 | TAGGGTGACTTGAAGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX7 | 402 | TAGGGTGACTTGAAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX7 | 403 | TAGGGTGACTTGAAGTTTTAGAGCTATGCTACGCATACGTGACGA |
| | | CGGGTTTTTTTTcagccaactccaaTTTTTTTTCCCGTCGTCACGTATGCGT |
| | | AGCA |
| PAX7 | 404 | ACGAGGTCGAGCCGGTTTTAGAGCTATAGCTACCGATGTCGAGTG |
| | | TAGGTTTTTTTTcagccaactccaaTTTTTTTTCCTACACTCGACATCGGTA |
| | | GCTA |
| PAX7 | 405 | ACGAGGTCGAGCCGGTTTTAGAGCTAATTACTCGAACGTTCCGCC |
| | | AGGGTTTTTTTTcagccaactccaaTTTTTTTTCCCTGGCGGAACGTTCGA |
| | | GTAAT |
| PAX7 | 406 | ACGAGGTCGAGCCGGTTTTAGAGCTAGCGCACGACCACTATCGTG |
| | | TAGGTTTTTTTTcagccaactccaaTTTTTTTTCCTACACGATAGTGGTCGT |
| | | GCGC |
| PAX7 | 407 | ACGAGGTCGAGCCGGTTTTAGAGCTAACTCGTTCGATAGAGAGTT |
| | | CGGGTTTTTTTTcagccaactccaaTTTTTTTTCCCGAACTCTCTATCGAAC |
| | | GAGT |
| PAX7 | 408 | ACGAGGTCGAGCCGGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX7 | 409 | ACGAGGTCGAGCCGGTTTTAGAGCTAGCTCAGTCGCGAATGAGCT |
| | | TTGGTTTTTTTTcagccaactccaaTTTTTTTTCCAAAGCTCATTCGCGACT |
| | | GAGC |
| PAX7 | 410 | ACGAGGTCGAGCCGGTTTTAGAGCTATAGCTCCCGTCCGTAGACG |
| | | TCGGTTTTTTTTcagccaactccaaTTTTTTTTCCGACGTCTACGGACGGGA |
| | | GCTA |
| PAX7 | 411 | ACGAGGTCGAGCCGGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX7 | 412 | ACGAGGTCGAGCCGGTTTTAGAGCTATGCTACGCATACGTGACGA |
| | | CGGGTTTTTTTTcagccaactccaaTTTTTTTTCCCGTCGTCACGTATGCGT |
| | | AGCA |
| PAX7 | 413 | GGCCCATTCATCAAGTTTTAGAGCTATAGCTACCGATGTCGAGTGT |
| | | AGGTTTTTTTTcagccaactccaaTTTTTTTTCCTACACTCGACATCGGTAG |
| | | CTA |
| PAX7 | 414 | GGCCCATTCATCAAGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX7 | 415 | GGCCCATTCATCAAGTTTTAGAGCTAGCGCACGACCACTATCGTGT |
| | | AGGTTTTTTTTcagccaactccaaTTTTTTTTCCTACACGATAGTGGTCGTG |
| | | CGC |
| PAX7 | 416 | GGCCCATTCATCAAGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| PAX7 | 417 | GGCCCATTCATCAAGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX7 | 418 | GGCCCATTCATCAAGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| PAX7 | 419 | GGCCCATTCATCAAGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX7 | 420 | GGCCCATTCATCAAGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX7 | 421 | GGCCCATTCATCAAGTTTTAGAGCTATGCTACGCATACGTGACGAC |
| | | GGGTTTTTTTTcagccaactccaaTTTTTTTTCCCGTCGTCACGTATGCGTA |
| | | GCA |
| PAX7 | 422 | GCTCCCTTGCGCCCGTTTTAGAGCTATAGCTACCGATGTCGAGTGT |
| | | AGGTTTTTTTTcagccaactccaaTTTTTTTTCCTACACTCGACATCGGTAG |
| | | CTA |
| PAX7 | 423 | GCTCCCTTGCGCCCGTTTTAGAGCTAATTACTCGAACGTTCCGCCA |
| | | GGGTTTTTTTTcagccaactccaaTTTTTTTTCCCTGGCGGAACGTTCGAGT |
| | | AAT |
| PAX7 | 424 | GCTCCCTTGCGCCCGTTTTAGAGCTAGCGCACGACCACTATCGTGT |
| | | AGGTTTTTTTTcagccaactccaaTTTTTTTTCCTACACGATAGTGGTCGTG |
| | | CGC |
| PAX7 | 425 | GCTCCCTTGCGCCCGTTTTAGAGCTAACTCGTTCGATAGAGAGTTC |
| | | GGGTTTTTTTTcagccaactccaaTTTTTTTTCCCGAACTCTCTATCGAACG |
| | | AGT |
| PAX7 | 426 | GCTCCCTTGCGCCCGTTTTAGAGCTACCTCCGTGTCGATCGTGCCA |
| | | TGGTTTTTTTTcagccaactccaaTTTTTTTTCCATGGCACGATCGACACGG |
| | | AGG |
| PAX7 | 427 | GCTCCCTTGCGCCCGTTTTAGAGCTAGCTCAGTCGCGAATGAGCTT |
| | | TGGTTTTTTTTcagccaactccaaTTTTTTTTCCAAAGCTCATTCGCGACTG |
| | | AGC |
| PAX7 | 428 | GCTCCCTTGCGCCCGTTTTAGAGCTATAGCTCCCGTCCGTAGACGT |
| | | CGGTTTTTTTTcagccaactccaaTTTTTTTTCCGACGTCTACGGACGGGA |
| | | GCTA |
| PAX7 | 429 | GCTCCCTTGCGCCCGTTTTAGAGCTATGCGTCGTCTACTACCTCTC |
| | | GGGTTTTTTTTcagccaactccaaTTTTTTTTCCCGAGAGGTAGTAGACGA |
| | | CGCA |
| PAX7 | 430 | GCTCCCTTGCGCCCGTTTTAGAGCTATGCTACGCATACGTGACGAC |
| | | GGGTTTTTTTTcagccaactccaaTTTTTTTTCCCGTCGTCACGTATGCGTA |
| | | GCA |

### EMBODIMENTS

The following non-limiting embodiments provide illustrative examples of the invention, but do not limit the scope of the invention.

Embodiment 1. A method for conversion of a plurality of pluripotent stem cells (PSCs) into a plurality of tissue-specific progenitor cells, comprising:
contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to modulate expression and/or activity level of a first target endogenous gene of the plurality of distinct target endogenous genes, wherein the first target endogenous gene comprises a member selected from the group consisting of a T-box transcription factor (TBX), a basic helix-loop-helix transcription factor (bHLH), and a Paired box transcription factor (PAX); and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises a different member selected from the group consisting of the TBX, the bHLH, and the PAX,
wherein, upon activation of the heterologous genetic circuit, the plurality of gate units operates to effect the conversion,
optionally wherein:
(1) the first target endogenous gene comprises the TBX,
further optionally wherein:
(a) the second target endogenous gene comprises the bHLH; and/or
   (b) the second target endogenous gene comprises the PAX; and/or
(2) the first target endogenous gene comprises the bHLH,
   further optionally wherein:
   (a) the second target endogenous gene comprises the PAX; and/or
(3) the TBX comprises one or more members selected from the group consisting of TBX1, TBX2, TBX3, TBX4, TBX5, TBX6, TBX10, TBX15, TBX18, TBX19, TBX20, TBX21, TBX22, and TBXT (Brachyury protein),
   further optionally wherein:
   (a) the TBX is TBXT; and/or
(4) the bHLH comprises one or more members selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXII, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2,
   further optionally wherein:
   (a) the bHLH is MSGN1; and/or
(5) the PAX comprises one or more members selected from the group consisting of PAX1, PAX2, PAX3, PAX4, PAX5, PAX6, PAX7, PAX8, and PAX9,
   further optionally wherein:
   (a) the PAX comprises PAX 3 or PAX7; and/or
   (b) the PAX comprises PAX3 and PAX7.

Embodiment 2. A method for conversion of a plurality of pluripotent stem cells (PSCs) into a plurality of tissue-specific progenitor cells, comprising:
contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to modulate expression and/or activity level of a first target endogenous gene of the plurality of distinct target endogenous genes, wherein the first target endogenous gene comprises a T-box transcription factor (TBX); and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises an additional TBX, wherein the TBX and the additional TBX are different types of TBX,
wherein, upon activation of the heterologous genetic circuit, the plurality of gate units operates to effect the conversion,
optionally wherein:
(1) the TBX or the additional TBX is selected from the group consisting of TBX1, TBX2, TBX3, TBX4, TBX5, TBX6, TBX10, TBX15, TBX18, TBX19, TBX20, TBX21, TBX22, and TBXT (Brachyury protein),
   further optionally wherein:
   (a) the TBX is TBXT; and/or
   (b) the additional TBX is TBX6.

Embodiment 3. A method for conversion of a plurality of pluripotent stem cells (PSCs) into a plurality of tissue-specific progenitor cells, comprising:
contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to reduce expression and/or activity level of a first target endogenous gene of the plurality of distinct target endogenous genes, wherein the first target endogenous gene comprises at least one cell de-differentiation factor; and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor,
wherein, upon activation of the heterologous genetic circuit, the plurality of gate units operates to effect the conversion,
optionally wherein:
(1) the at least one cell de-differentiation factor is a transcription factor; and/or
(2) the at least one cell de-differentiation factor comprises one or more members selected from the group consisting of Oct4, Sox2, Klf4, and c-Myc,
   further optionally wherein:
   (a) the at least one cell-differentiation factor is Oct4; and/or
(3) the at least one tissue-specific differentiation factor comprises one or more members selected from the group consisting of a T-box transcription factor (TBX), a basic helix-loop-helix transcription factor (bHLH), and a Paired box transcription factor (PAX); and/or
   further optionally wherein:
   (a) the at least one tissue-specific differentiation factor comprises two or more members selected from the group consisting of the TBX, the bHLH, and the PAX,
      further optionally wherein:
      (a.1) expression and/or activity levels of the two or more members are modulated sequentially; and/or
      (a.2) (i) expression and/or activity level of the TBX or the bHLH is modulated prior to (ii) expression and/or activity level of the PAX; and/or
   (b) the TBX comprises one or more members selected from the group consisting of TBX1, TBX2, TBX3, TBX4, TBX5, TBX6, TBX10, TBX15, TBX18, TBX19, TBX20, TBX21, TBX22, and TBXT (Brachyury protein),
      further optionally wherein:
      (b.1) the TBX is TBXT; and/or
   (c) bHLH comprises one or more members selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2,
      further optionally wherein:
      (i) the bHLH is MSGN1; and/or
   (d) the PAX comprises one or more members selected from the group consisting of PAX1, PAX2, PAX3, PAX4, PAX5, PAX6, PAX7, PAX8, and PAX9,
      further optionally wherein:
      (i) the PAX comprises PAX 3 or PAX7.
      (ii) the PAX comprises PAX3 and PAX7.

Embodiment 4. The method of any one of Embodiments 1-3, optionally wherein,
(1) the plurality of tissue-specific progenitor cells comprises muscle stem cells (satellite cells), further optionally wherein:
   (a) the muscle stem cells exhibit higher expression levels of two or more muscle stem cell markers, as compared to control PSCs, wherein the two or more muscle stem cell markers are selected from the group consisting of CD271, ERBB3, CD54, ITGA9, and SDC2,
      further optionally wherein:
      (a.1) the two or more muscle stem cell markers are (i) CD271 and ERBB3, (ii) CD54 and ITGA9, (iii) SDC2 and ITGA9, or (iv) SDC2 and CD54; and/or
(2) the plurality of tissue-specific progenitor cells are not myoblasts; and/or
(3) the plurality of tissue-specific progenitor cells substantially do not express Myf5 or MyoD; and/or (4) the plurality of tissue-specific progenitor cells is substantially mitotically dormant; and/or (5) reduction of the expression and/or activity level of the first target endogenous gene occurs prior to modulation of the expression and/or activity level of the second target endogenous gene; and/or (6) the second gate unit is preconfigured to enhance expression and/or activity level of the second target endogenous gene; and/or
(7) the conversion occurs in conditions substantially free of serum and/or exogenous cell differentiation regulatory factors; and/or
(8) the activation occurs upon contact with an activating moiety,
   further optionally wherein:
   (a) the activating moiety comprises a gNA capable of forming a complex with an endonuclease; and/or
(9) the first gate unit and the second gate unit each comprise a guide nucleic acid (gNA) that is activatable,
   further optionally wherein:
   (a) the gNA comprises a spacer sequence,
      further optionally wherein:
      (i) the spacer sequence comprises one of any of SEQ ID NO: 1-31; and/or
      (ii) the spacer sequence comprises a polynucleotide sequence exhibiting at least about 80% sequence identity to the polynucleotide sequence selected from the group consisting of SEQ ID NO: 1-31; and/or
(10) the endonuclease comprises a CRISPR/Cas protein,
   further optionally wherein:
   (a) the CRISPR/Cas protein is a deactivated Cas (dCas) protein; and/or
   (b) the CRISPR/Cas protein is Cas9; and/or
(11) the activatable gNA comprises a self-cleaving gNA; and/or
(12) the method further comprising storing the plurality of tissue-specific progenitor cells in a sterile vial; and/or
(13) the method further comprising storing the plurality of tissue-specific progenitor cells at a temperature of at most about 4 degrees Celsius (° C.); and/or
(14) the method further comprising storing the plurality of tissue-specific progenitor cells at a temperature of at most about −80 degrees Celsius (° C.); and/or
(15) the method further comprising storing the plurality of tissue-specific progenitor cells at a temperature of at most about −190 degrees Celsius (° C.); and/or
(16) the method further comprising administering at least one of the plurality of tissue-specific progenitor cells to a subject in need thereof; and/or
(17) the first gate unit and the second gate unit each comprises a polynucleotide sequence encoding the gNA, wherein the polynucleotides sequence comprises a polyT sequence configured to disrupt expression of a functional form of the gNA.

Embodiment 5. A system for conversion of a plurality of pluripotent stem cells (PSCs) into a plurality of tissue-specific progenitor cells, comprising:
a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to modulate expression and/or activity level of a first target endogenous gene of the plurality of distinct target endogenous genes, wherein the first target endogenous gene comprises a member selected from the group consisting of a T-box transcription factor (TBX), a basic helix-loop-helix transcription factor (bHLH), and a Paired box transcription factor (PAX); and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises a different member selected from the group consisting of the TBX, the bHLH, and the PAX,
wherein, upon activation of the heterologous genetic circuit, the plurality of gate units operates to effect the conversion,
   optionally wherein:
   (1) the first target endogenous gene comprises the TBX,
      further optionally wherein:
      (a) the second target endogenous gene comprises the bHLH; and/or
      (b) the second target endogenous gene comprises the PAX; and/or
   (2) the first target endogenous gene comprises the bHLH,
      further optionally wherein:
      (a) the second target endogenous gene comprises the PAX; and/or
   (3) the TBX comprises one or more members selected from the group consisting of TBX1, TBX2, TBX3, TBX4, TBX5, TBX6, TBX10, TBX15, TBX18, TBX19, TBX20, TBX21, TBX22, and TBXT (Brachyury protein),
      further optionally wherein:
      (a) the TBX is TBXT; and/or
   (4) the bHLH comprises one or more members selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2,
      further optionally wherein:
      (a) the bHLH is MSGN1; and/or
   (5) the PAX comprises one or more members selected from the group consisting of PAX1, PAX2, PAX3, PAX4, PAX5, PAX6, PAX7, PAX8, and PAX9,
      further optionally wherein:
      (a) the PAX comprises PAX 3 or PAX7; and/or
      (b) the PAX comprises PAX3 and PAX7.

Embodiment 6. A system for conversion of a plurality of pluripotent stem cells (PSCs) into a plurality of tissue-specific progenitor cells, comprising:
a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to modulate expression and/or activity level of a first target endogenous gene of the plurality of distinct target endogenous genes, wherein the first target endogenous gene comprises a T-box transcription factor (TBX); and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises an additional TBX, wherein the TBX and the additional TBX are different types of TBX,
wherein, upon activation of the heterologous genetic circuit, the plurality of gate units operates to effect the conversion,
   optionally wherein:
   (1) the TBX or the additional TBX is selected from the group consisting of TBX1, TBX2, TBX3, TBX4, TBX5, TBX6, TBX10, TBX15, TBX18, TBX19, TBX20, TBX21, TBX22, and TBXT (Brachyury protein),
further optionally wherein:
(a) the TBX is TBXT; and/or
(b) the additional TBX is TBX6.

Embodiment 7. A system for conversion of a plurality of pluripotent stem cells (PSCs) into a plurality of tissue-specific progenitor cells, comprising:
a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
   i) a first gate unit that is preconfigured to reduce expression and/or activity level of a first target endogenous gene of the plurality of distinct target endogenous genes, wherein the first target endogenous gene comprises at least one cell de-differentiation factor; and
   ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor,
wherein, upon activation of the heterologous genetic circuit, the plurality of gate units operates to effect the conversion, optionally wherein:
   (1) the at least one cell de-differentiation factor is a transcription factor; and/or
   (2) the at least one cell de-differentiation factor comprises one or more members selected from the group consisting of Oct4, Sox2, Klf4, and c-Myc,
further optionally wherein:
(a) the at least one cell-differentiation factor is Oct4; and/or
   (3) the at least one tissue-specific differentiation factor comprises one or more members selected from the group consisting of a T-box transcription factor (TBX), a basic helix-loop-helix transcription factor (bHLH), and a Paired box transcription factor (PAX),
further optionally wherein:
(a) the at least one tissue-specific differentiation factor comprises two or more members selected from the group consisting of the TBX, the bHLH, and the PAX,
   further optionally wherein:
   (a.1) expression and/or activity levels of the two or more members are modulated sequentially; and/or
   (a.2) (i) expression and/or activity level of the TBX or the bHLH is modulated prior to (ii) expression and/or activity level of the PAX; and/or
(b) the TBX comprises one or more members selected from the group consisting of TBX1, TBX2, TBX3, TBX4, TBX5, TBX6, TBX10, TBX15, TBX18, TBX19, TBX20, TBX21, TBX22, and TBXT (Brachyury protein),
   further optionally wherein:
   (b.1) the TBX is TBXT; and/or
(c) bHLH comprises one or more members selected from the group consisting of AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2,
   further optionally wherein:
   (c.1) the bHLH is MSGN1; and/or
(d) the PAX comprises one or more members selected from the group consisting of PAX1, PAX2, PAX3, PAX4, PAX5, PAX6, PAX7, PAX8, and PAX9,
   further optionally wherein:
   (d.1) the PAX comprises PAX 3 or PAX7; and/or
   (d.2) the PAX comprises PAX3 and PAX7.

Embodiment 8. The system of any one of Embodiments 5-7, optionally wherein:
(1) the plurality of tissue-specific progenitor cells comprises muscle stem cells (satellite cells),
   Further optionally wherein:
   (a) the muscle stem cells exhibit higher expression levels of two or more muscle stem cell markers, as compared to control PSCs, wherein the two or more muscle stem cell markers are selected from the group consisting of CD271, ERBB3, CD54, ITGA9, and SDC2,
      Further optionally wherein:
      (a.1) the two or more muscle stem cell markers are (i) CD271 and ERBB3, (ii) CD54 and ITGA9, (iii) SDC2 and ITGA9, or (iv) SDC2 and CD54; and/or
(2) the plurality of tissue-specific progenitor cells are not myoblasts; and/or
(3) the plurality of tissue-specific progenitor cells substantially do not express Myf5 or MyoD; and/or
(4) the plurality of tissue-specific progenitor cells is substantially mitotically dormant; and/or
(5) reduction of the expression and/or activity level of the first target endogenous gene occurs prior to modulation of the expression and/or activity level of the second target endogenous gene; and/or
(6) the second gate unit is preconfigured to enhance expression and/or activity level of the second target endogenous gene; and/or
(7) the conversion occurs in conditions substantially free of serum and/or exogenous cell differentiation regulatory factors; and/or
(8) the activation occurs upon contact with an activating moiety,
   further optionally wherein:
   (a) the activating moiety comprises a gNA capable of forming a complex with an endonuclease; and/or
(9) the first gate unit and the second gate unit each comprise a guide nucleic acid (gNA) that is activatable,
   further optionally wherein:
   (a) the gNA comprises a spacer sequence,
      further optionally wherein:
      (i) the spacer sequence comprises one of any of SEQ ID NO: 1-31; and/or
      (ii) the spacer sequence comprises a polynucleotide sequence exhibiting at least about 80% sequence identity to the polynucleotide sequence selected from the group consisting of SEQ ID NO: 1-31; and/or
(10) the endonuclease comprises a CRISPR/Cas protein,
   further optionally wherein:
   (a) the CRISPR/Cas protein is a deactivated Cas (dCas) protein; and/or
   (b) the CRISPR/Cas protein is Cas9; and/or
(11) the activatable gNA comprises a self-cleaving gNA; and/or
(12) the first gate unit and the second gate unit each comprises a polynucleotide sequence encoding the gNA, wherein the polynucleotides sequence comprises a polyT sequence configured to disrupt expression of a functional form of the gNA

Additional details of heterologous genetic circuits (HGC) and uses thereof are provided in International Application No. PCT/US2018/052211 (entitled “CRISPR/CAS SYSTEM AND METHOD FOR GENOME EDITING AND MODULATING TRANSCRIPTION”), International Application No. PCT/US2023/013240 (entitled “SYSTEMS FOR CELL PROGRAMMING AND METHODS THEREOF), International Application No. PCT/US2023/028169 (entitled “SYSTEMS FOR CELL PROGRAMMING AND METHODS THEREOF”), and Clarke et al., Molecular Cell, 81, 226-238, 2021 (entitled “Sequential Activation of Guide RNAs to Enable Successive CRISPR-Cas9 Activities”), each of which is incorporated herein by reference in its entirety.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-72. (canceled)

73. A method for conversion of a plurality of pluripotent stem cells (PSCs) into a plurality of tissue-specific progenitor cells, comprising:
contacting the plurality of PSCs with a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
i) a first gate unit that is preconfigured to reduce expression and/or activity level of a first target endogenous gene of the plurality of distinct target endogenous genes, wherein the first target endogenous gene comprises at least one cell de-differentiation factor; and
ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor,
wherein, upon activation of the heterologous genetic circuit, the plurality of gate units operates to effect the conversion.

74. The method of claim 73, wherein the activation of the heterologous genetic circuit is a single activation.

75. The method of claim 73, wherein the at least one cell de-differentiation factor is a transcription factor.

76. The method of claim 73, wherein the at least one cell de-differentiation factor comprises one or more members selected from the group consisting of Oct4, Sox2, Klf4, and c-Myc.

77. The method of claim 76, wherein the at least one cell-differentiation factor is Oct4.

78. The method of claim 73, wherein the at least one tissue-specific differentiation factor comprises one or more members selected from the group consisting of a T-box transcription factor (TBX), a basic helix-loop-helix transcription factor (bHLH), and a Paired box transcription factor (PAX).

79. The method of claim 78, wherein the at least one tissue-specific differentiation factor comprises two or more members selected from the group consisting of the TBX, the bHLH, and the PAX.

80. The method of claim 79, wherein expression and/or activity levels of the two or more members are modulated sequentially.

81. The method of claim 79, wherein (i) expression and/or activity level of the TBX or the bHLH is modulated prior to (ii) expression and/or activity level of the PAX.

82. The method of claim 78, wherein the TBX comprises one or more members selected from the group consisting of TBX1, TBX2, TBX3, TBX4, TBX5, TBX6, TBX10, TBX15, TBX18, TBX19, TBX20, TBX21, TBX22, and TBXT (Brachyury protein).

83. The method of claim 82, wherein the TBX is TBXT.

84. The method of claim 79, wherein the bHLH is MSGN1.

85. The method of claim 78, wherein the PAX comprises one or more members selected from the group consisting of PAX1, PAX2, PAX3, PAX4, PAX5, PAX6, PAX7, PAX8, and PAX9.

86. The method of claim 73, wherein the plurality of tissue-specific progenitor cells comprises muscle stem cells (satellite cells).

87. The method of claim 86, wherein the muscle stem cells comprise two or more muscle stem cell markers selected from the group consisting of (i) CD271 and ERBB3, (ii) CD54 and ITGA9, (iii) SDC2 and ITGA9, or (iv) SDC2 and CD54.

88. The method of claim 73, wherein the first gate unit and the second gate unit each comprise a guide nucleic acid (gNA) that is activatable.

89. The method of claim 88, wherein the gNA comprises a spacer sequence.

90. The method of claim 89, wherein the spacer sequence comprises a polynucleotide sequence exhibiting at least about 80% sequence identity to the polynucleotide sequence selected from the group consisting of SEQ ID NO: 1-31.

91. A system for conversion of a plurality of pluripotent stem cells (PSCs) into a plurality of tissue-specific progenitor cells, comprising:
a heterologous genetic circuit comprising a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
i) a first gate unit that is preconfigured to reduce expression and/or activity level of a first target endogenous gene of the plurality of distinct target endogenous genes, wherein the first target endogenous gene comprises at least one cell de-differentiation factor; and
ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor,
wherein, upon activation of the heterologous genetic circuit, the plurality of gate units operates to effect the conversion.

92. A method of treating a subject in need thereof, the method comprising administering to the subject a plurality of cells generated via activation of a heterologous genetic circuit in a plurality of pluripotent stem cells (PSCs) to effect conversion of the plurality of PSCs into a plurality of tissue-specific progenitor cells,
wherein the heterologous genetic circuit comprises a plurality of gate units, wherein the heterologous genetic circuit is activatable to induce the plurality of gate units to modulate expression and/or activity levels of a plurality of distinct target endogenous genes in a sequential manner to effect the conversion, wherein each of the plurality of gate units is necessary but not individually sufficient to effect the conversion, and wherein the plurality of gate units comprises:
(i) a first gate unit that is preconfigured to reduce expression and/or activity level of a first target endogenous gene of the plurality of distinct target endogenous genes, wherein the first target endogenous gene comprises at least one cell de-differentiation factor; and
(ii) a second gate unit that is preconfigured to modulate expression and/or activity level of a second target endogenous gene of the plurality of distinct target endogenous genes, such that the expression and/or levels of the first target gene and the second target gene are modulated in the sequential manner, wherein the second target endogenous gene comprises at least one tissue-specific differentiation factor,
wherein, upon activation of the heterologous genetic circuit, the plurality of gate units operates to effect the conversion.
